SEQUENCE LISTING (59 sequences)

SEQ ID NO: 1 - RNA, 21 nt, synthetic construct
  source 1..21: mol_type = other RNA
gcaacagcgtaaaaattgttt

SEQ ID NO: 2 - RNA, 21 nt, synthetic construct
  source 1..21: mol_type = other RNA
cggtgtccacagtccttgatt

SEQ ID NO: 3 - RNA, 21 nt, synthetic construct
  source 1..21: mol_type = other RNA
caagaaaagttataagttttt

SEQ ID NO: 4 - RNA, 21 nt, synthetic construct
  source 1..21: mol_type = other RNA
cggaggaagttacagatgttt

SEQ ID NO: 5 - RNA, 21 nt, synthetic construct
  source 1..21: mol_type = other RNA
agatgcaagaggtaccaaatt

SEQ ID NO: 6 - RNA, 21 nt, synthetic construct
  source 1..21: mol_type = other RNA
ggtgtcggatgatttatcatt

SEQ ID NO: 7 - RNA, 21 nt, synthetic construct
  source 1..21: mol_type = other RNA
gaagctagaacttaccaaatt

SEQ ID NO: 8 - RNA, 21 nt, synthetic construct
  source 1..21: mol_type = other RNA
ggatgtctttcctaggagatt

SEQ ID NO: 9 - RNA, 21 nt, synthetic construct
  source 1..21: mol_type = other RNA
cgctcatcgacaataattatt

SEQ ID NO: 10 - RNA, 21 nt, synthetic construct
  source 1..21: mol_type = other RNA
cactttagatgtatctaattt

SEQ ID NO: 11 - RNA, 21 nt, synthetic construct
  source 1..21: mol_type = other RNA
ggagcataaagtagaccgatt

SEQ ID NO: 12 - RNA, 20 nt, synthetic construct
  source 1..20: mol_type = other RNA
ttgccactacagctaccacc

SEQ ID NO: 13 - RNA, 20 nt, synthetic construct
  source 1..20: mol_type = other RNA
ccaatgtactcagggaaaca

SEQ ID NO: 14 - RNA, 20 nt, synthetic construct
  source 1..20: mol_type = other RNA
agtccaccaaagtgtttgct

SEQ ID NO: 15 - RNA, 20 nt, synthetic construct
  source 1..20: mol_type = other RNA
aaaggagtctgcttacttac

SEQ ID NO: 16 - DNA, 20 bp, Homo sapiens
  source 1..20: mol_type = genomic DNA
ttgccactacagctaccacc

SEQ ID NO: 17 - DNA, 20 bp, Homo sapiens
  source 1..20: mol_type = genomic DNA
ccaatgtactcagggaaaca

SEQ ID NO: 18 - DNA, 20 bp, Homo sapiens
  source 1..20: mol_type = genomic DNA
agtccaccaaagtgtttgct

SEQ ID NO: 19 - DNA, 20 bp, Homo sapiens
  source 1..20: mol_type = genomic DNA
aaaggagtctgcttacttac

SEQ ID NO: 20 - DNA, 20 bp, Homo sapiens
  source 1..20: mol_type = genomic DNA
cttctcgtaatggcagatct

SEQ ID NO: 21 - DNA, 20 bp, Homo sapiens
  source 1..20: mol_type = genomic DNA
gcacttgaggatcttgcacg

SEQ ID NO: 22 - DNA, 20 bp, Homo sapiens
  source 1..20: mol_type = genomic DNA
gaggtcctctatgccatgga

SEQ ID NO: 23 - DNA, 20 bp, Homo sapiens
  source 1..20: mol_type = genomic DNA
ccatacccatccatccagct

SEQ ID NO: 24 - DNA, 20 bp, Homo sapiens
  source 1..20: mol_type = genomic DNA
cccatgtgaaggaccttcaa

SEQ ID NO: 25 - DNA, 20 bp, Homo sapiens
  source 1..20: mol_type = genomic DNA
gttcttcaaactctgttcgg

SEQ ID NO: 26 - DNA, 20 bp, Homo sapiens
  source 1..20: mol_type = genomic DNA
gaaggcgtagacttatctga

SEQ ID NO: 27 - DNA, 20 bp, Homo sapiens
  source 1..20: mol_type = genomic DNA
agccaacttccagcaccaat

SEQ ID NO: 28 - DNA, 20 bp, Homo sapiens
  source 1..20: mol_type = genomic DNA
gggcaatggaccttctgcct

SEQ ID NO: 29 - DNA, 20 bp, Homo sapiens
  source 1..20: mol_type = genomic DNA
ggctgcggggcagagggcaa

SEQ ID NO: 30 - DNA, 20 bp, Homo sapiens
  source 1..20: mol_type = genomic DNA
cgggcaggctgcggggcaga

SEQ ID NO: 31 - DNA, 20 bp, Homo sapiens
  source 1..20: mol_type = genomic DNA
acgggcaggctgcggggcag

SEQ ID NO: 32 - RNA, 21 nt, synthetic construct
  source 1..21: mol_type = other RNA
acaatttttacgctgttgcca

SEQ ID NO: 33 - RNA, 21 nt, synthetic construct
  source 1..21: mol_type = other RNA
tcaaggactgtggacaccggt

SEQ ID NO: 34 - RNA, 21 nt, synthetic construct
  source 1..21: mol_type = other RNA
aaacttataacttttcttgga

SEQ ID NO: 35 - RNA, 21 nt, synthetic construct
  source 1..21: mol_type = other RNA
acatctgtaacttcctccgca

SEQ ID NO: 36 - RNA, 21 nt, synthetic construct
  source 1..21: mol_type = other RNA
tttggtacctcttgcatctca

SEQ ID NO: 37 - RNA, 21 nt, synthetic construct
  source 1..21: mol_type = other RNA
tgataaatcatccgacacctg

SEQ ID NO: 38 - RNA, 21 nt, synthetic construct
  source 1..21: mol_type = other RNA
tttggtaagttctagcttcct

SEQ ID NO: 39 - RNA, 21 nt, synthetic construct
  source 1..21: mol_type = other RNA
tctcctaggaaagacatccaa

SEQ ID NO: 40 - RNA, 21 nt, synthetic construct
  source 1..21: mol_type = other RNA
taattattgtcgatgagcggc

SEQ ID NO: 41 - RNA, 21 nt, synthetic construct
  source 1..21: mol_type = other RNA
attagatacatctaaagtggg

SEQ ID NO: 42 - RNA, 21 nt, synthetic construct
  source 1..21: mol_type = other RNA
tcggtctactttatgctcctt

SEQ ID NO: 43 - RNA, 3610 nt, Homo sapiens
  source 1..3610: mol_type = mRNA
cttcctgcggctgaaccgcccggctgagccgacattgccggcgtcttggcgattcggcccgacgagctccgctttcgctacagcatggtggcctactggagacaggctggactcagctacatccgatactcccagatctgtgcaaaagcagtgagagatgcactgaagacagaattcaaagcaaatgctgagaagacttctggcagcaacgtaaaaattgtgaaagtaaagaaggaataatctaccctgactaaagcttgaaatgctacatttccaaggtgaagatgtgtgggcacatgttatggcagattgaaaaggatctcattccatgggaaaaaaaaaaatcctgtcttgttcataaattgacaatgtcaataaattgaaatatggttcactgttactcttgattttagtttgtgagactttttcattttctttaaaggaactgtctgcacagctgcatgattatggccatgtgaaccagggtttcttaaaagcaggggaagccagatttaatatttagtgaccaaagggatacagaaagcattttgaaaatgtctcaaaatagttgtttcatgacattaattggtgatcaaaattatctgctgtcattgaggcttcgtagtcccttatttttgaattgttgtcatgtcagaataatgacatggttatcagtaacaaatggaaataattattaaatactaaaagtgtagattagaagaagaaatactaaattgtacttgttttgtattgtgcacttaaagcctatagataagaacggttacagaatttaagcacaaagtgtttttcacattgttttgtaaatgaatctttatactctggtagcatttctcataggaaatgaggcaaagctattgaataacttgctcattgagttgatggccaaactgaaattaaaacctaggagttttgactgcaagtctgggacacggtgaccacaacagcagtttcgtggtatttccatagtgattcattcattcattatttagggatgaaaaattgttgcaagttatgtttttatctttctttgaaacttgaaagggcttgggcaagtaaaacaccattgcaaagttaactgcctgattttaactgtttttggagactggagaaataaatttgaactggaaattttgaaacaccattctaggatcagttctcacatggtacttaagctcacaatgatttatcagggccactgtcagcaatgccgaggataagccgaaaagaacatctttgttctcggcttctgggaatgacttattgtcccgacacttgacctgtaggttttactgaattctcagcacagagcgagcagggaggcagagccaattgagtggaagagggtgcctctggaaaacagcaggttaggagcccttggagtcctccgcctcggagctgtcatcctgctgttggcaagttagggcacctcagttcagtgctgtggcccccaagattaaggtactaggaagtatcctccattacagggaacaggaataaatgaatcaaaaagctatcgggcctttttgacatgggacgaataaagtaactaataggcaaaggggctaaatgatttccagtcaagtaattagagataagtcatttattaaggaggcagatggggtattattggtatatagatggcatagtagattgctagtatattggtagattattggtatatagatggtatgttaaggaggcagatggggtattactggtatatagatggtatggtagattggtagtatattggcagattattggtatatggtatgttgaggcagatggggtattactggtatatagatggcatagtagattggtagtatattggtagattattggtatagagatggtatattattaaggaggcagatggggtaattagaggtgagacttagggacagggcttgtgatatggaaaggagtgaagtcactgagtaaaagacaagtttactgtcagcagagattcttagaggctcttttctccgtgattaaaaaaaaatcctaaaccagtagtataataaacttcctgcctccagcagtgctgggtagctggcaggatgactgtgcccagtgatgaggtgaccccagtcagaccaacttaatgagtatcccactaattagttggatgatgtttgaagctgtcagttaatctcttagttcacatcgttcttgtgagcaaagtgaaggaatcggataagctctaaatttcggtgatgctgctgctctgagagaccaaggtcgctggagcaaccgtactgctaaaggataagctcttcaaaatcaaaagggacagatttcttaagtttctagtttgttttgtgtttttacatctatttctaccatacgaaagagtaggatggtgagatgcatttctaaatatttttaactactcagtctggatggttaggcttcaagaaaaggaatttatttagtctgaaataaagcaattttgccatttaaaattgaattactttgtagtttaatgattactattgagcaaacaaatctttacagtaatataagtcagcaaaagttttttaaaaggtttaaatccctccttccttacatctatttcattgcttttttaggtttgggaagtaaagcaccagaggaagcatgtgtaaatccttgtataatcgcaggctgtaagtctttagcatctcaggaagttactaacttcaaactaagtatataggtagagtttcttactaaatctagtgcttcttgaaccacaagtagaaagcatttaaaacatgaatgttgttttgtgttttttgaagtttgtaaatagaagacttgttgatgatccgatggcaaggtatttttctcttggtatgtattttagttatttcctcgtgatgcataagtgaaaagagtgaagtttctcagaatgagcaactgtcatccatctacctgctattttattattgctgattacaaaagcaaatcaagagatgagaacccagttgcctgcaagtaaatatttactgcattgagggtcggagcattttcccattaccggttatccatggatcaaatagtgtatctcagtggtaattctagagggccattaaaaccctgatggtgctggaagagatggcagtgctgcatgtcagaaataggtaaactgtaattaagaagttacagatgatttgattacgctcttgtgtatttggtcctgttataatgtgagcagattaaaatcatgtagtgcttaaagctatgtcattatgcaaacatttatggcaacttctgcaaattaatttgaactgtaaaagttccctaatgagacgatgtcctccataactgagaaggacactgcagccattgctgcttaattcctgcatttgaatggttgccgagcgcatggtgtgccctttctcaaagagcagagaggctgcagagcattttggggggtgatgactgaatgaaagtgaaaagaccatttcaaaatagcctttaaaaggaaagccaaggatgattattaatgaattcctgggatctaacagtagtaagaaatttaaactatataaacatatatgttaaaggctagctcttcagaaataaagatcagaagactgca

SEQ ID NO: 44 - DNA, 568 bp, Homo sapiens
  source 1..568: mol_type = genomic DNA
gaacccgggaggtggagggtgcagtgagccaagatcacatcactgcacttcagcctgggcaacaagagtaaaactccgtctaaaaaaaaagaaaaaaaaggacattgccagagtcttggcgatttggcccgacgcgctccgctttcgctacagcatggtggcctactggagacaggctggactcagctacatccgatactcccagatctgtgcaaaagtagtgagagatgcactgaagacagaattcaaagcaaatgccaaaaagacttctggcaacagcgtaaaaattgtgaaagtaaagaaggaataatctaccctgactaaagcttgaaatgctgcatttccaaggtgaagatgtatgggcacatgttatggcagattgaaaaggatctcatttcatgggaaaaaaaaatcctgtcttgttcataaattgacaatgtcaataaattgaaatatggttcactgttaaaaaaaaaaaaaaaaaaggttggaaagtgcaaggcaaggaaatggattctctcccagaaacctccagaaggaaccagccctgccaacaccttgattttag

SEQ ID NO: 45 - RNA, 3013 nt, Homo sapiens
  source 1..3013: mol_type = mRNA
cattgccctctgccccgcagcctgcccgttcccaggccagcgagggcgctcccaggccggtctgcagccagaacacagcccgttccatgtgcttgtcctcgggacacattaggaatgaacccaggcctcagtcctggcctcctcctcaaggctgggtgctcagacgtccccctcagcccctttctctagaccttccggctgaacaaacgagaacatctcacagccctgcaacagccaggagctgctcgtggagctccacgcccggctggagagagaggaggcaccctgggcagcccgccagcacccaggctctcacagtggaagcccacgtcttcttgggctctattttgaaagctgttctttccacgacggctttgacaagaatcgatttgagtgagccggcgtgtcaatacaggccagcacaggactggcctcagggccacattggggagggtggcgaccaggccctggctgcagcccacgtgggcaaaagcatggccaggcaggaggtggggcctgtgagcttggctacagcgaccgtgggcgctgggcccggggctcctcagccctgaggggagacccaggcccaggcaggctcccaggcgggctcccgggctcccagccttccgatcttctccctttgagaccttgccttctgcacagtgacaggaaagagccctgagtcccactttgcagggatgtattgcatttccagaaagttccttccgggtgcagagtgatacatgaaccctggggactcactccaacttggtattgattttctccacggaggacccagcagtcacggcccgtgtctgcacagccccttggatcctgcggaggaattcagccggcccctccccctgtgccgaaaaccacggaggcaaagccaggccagggtacagtgagaacacgggagaggactgagagaccgctggctgccgagggaggaagagcaggattcgcacccccgtcatcgctgggcccacacgccgccgacagcttccgcgtgtggccatctgtctttcagaaactctctggcagagggacccagcagatgacacgggggccgttcacactctgaaacctgcctggagtcaaaacactgctggcgagcctccaccgcaaacccaatccagatggtcttccctggtggccctcggggtggcgggcaccctgcccactccctgccagcccctgcccgctggactccaggcccggtcgagcccccgaccggtctcctgacccagcatgctgctctcccctgggcccggcttcctcgcccgctgggactcccaggcccctccagcccagccccgagagcaggagccccaagggaggcttggcggctgtggaggcgagtgggccacgtgtagctgggaggccggcaggaccaggagcccctcacagcgcagcacccacagcatcacgagccacttccactcctgggtggcctgggggcccccaccctgctctgagctgccccttgttgtgacctggggtcccccgtggtgggcccgccccttgagcctgggatccagcctcagcgaggctccagaagtgtcgtctccgccggccaggcgtcagcgcctccacccaggatcacacactcctatctctgcacttctgccctccatcctgctccacgccgcctctgcttgttattccagcacattccagggtgatgcctgctgaagccacgcagccagtgcctccaccctaccccccatctgacccaccggccccttccgcacgctggaagcctgcccggcctgaagtgtcctcacctgtcgccctatcctcccctccactccagcccaccggtgactcagccctcacagctctctcctccccgagggagccggtgacttggctctgatggtgccgggacccagccccggcatgttcagaatccttgtcctttaacctgcgtccacatgtagtcgcctccacctccctgcctgaacctccaggccctgcagagcaaacactgctgcctgcaggagacccctggggccttctccgtcccctcatcctcctaaggagcctcccctgccaccgtgccagaatgtgccggaggaacagaggcccgagcctcccgtggccccagcgaggcttcctctggggaagacagtgagcccaacgccctggctctcgaggggtggacggagtccagctggcatcaaccgggtgcccctcttcaccggtgtccacagtccttgagagcaaagggttcccagaggcgacctccctcctgcacaggcatttgtcatccttaccctctaggacagctttgccatgtgcagtgaacccctggacagtgttgcgtgccactttgccggatttggactgtatacagaccgaagcatgtggcatgaagccttctgctgtcagcaagttccttgtccctcgacagtatgtgcccaggctgcccttcaaggtggcagtgatggcagttgtctcttgcacgctgcgtacagttctgtcactgactcttacacacagccctggagacgggagagtgccgggagcattctatgcaagtgtcctggcctttgcgggcgtctccacagcacaaacacgagtcagggaccacccctcttctgcttccatgattccaccaactgggtggggcagagcagtgccagggccggaggcacgccccaggtgagggcaggtggccatgccagtgagtgcactgcgttctcagttaattcatccgaaatatttctaagtttcttgattattgcagttgcatccccaagataatgccgccctcaccaagtagaaggcacatgtgctctgatggcccctcccacactggggtcagggcacagcaacttagcgaggcgacgtgggggctcagaggacctcggaccaggctcggcacatggggcttctccagccagccagggggaagcaccctgcagactgctaacttttgcaaaatagagtaaaaaagatcagctagaaaaataaaacaaaaaaggaaagatatacaaaatacaaggcc

SEQ ID NO: 46 - RNA, 3015 nt, Homo sapiens
  source 1..3015: mol_type = unassigned RNA
cattgccctctgccccgcagcctgcccgttcccaggccagcgagggcgctcccaggccggtctgcagccagaacacagcccgttccatgtgcttgtcctcgggacacattaggaatgaacccaggcctcagtcctggcctcctcctcaaggctgggtgctcagacgtccccctcagcccctttctctagaccttccggctgaacaaacgagaacatctcacagccctgcaacagccaggagctgctcgtggagctccacgcccggctggagagagaggaggcaccctgggcagcccgccagcacccaggctctcacagtggaagcccacgtcttcttgggctctattttgaaagctgttctttccacgacggctttgacaagaatcgatttgagtgagccggcgtgtcaatacaggccagcacaggactggcctcagggccacattggggagggtggcgaccaggccctggctgcagcccacgtgggcaaaagcatggccaggcaggaggtggggcctgtgagcttggctacagcgaccgtgggcgctgggcccggggctcctcagccctgaggggagacccaggcccaggcaggctcccaggcgggctcccgggctcccagccttccgatcttctccctttgagaccttgccttctgcacagtgacaggaaagagccctgagtcccactttgcagggatgtattgcatttccagaaagttccttccgggtgcagagtgatacatgaaccctggggactcactccaacttggtattgattttctccacggaggacccagcagtcacggcccgtgtctgcacagccccttggatcctgcggaggaattcagccggcccctccccctgtgccgaaaaccacggaggcaaagccaggccagggtacagtgagaacacgggagaggactgagagaccgctggctgccgagggaggaagagcaggattcgcacccccgtcatcgctgggcccacacgccgccgacagcttccgcgtgtggccatctgtctttcagaaaccctctggcagagggacccagcagatgacacgggggccgttcacactctgaaacctgcctggagtcaaaacactgctggcgagcctccaccgcaaacccaatccagatggtcttccctggtggccctcggggtggcgggcaccctgcccactccctgccagcccctgcccgctggactccaggcccggccgagcccccgaccggtctcctgacccagcatgctgctctcccctgggcccggctttctcgcccgctgggactcccaggcccctccagcccagccccgagagcaggagccccaagggaggcttggcggctgtggaggcgagtgggccacgtgtagctgggaggccggcaggaccaggagcccctcacagcgcagcacccacagcatcacgagccacttccactcctgggtggcctgggggcccccaccctgctctgagctgccccttgttgtgacctggggtcccccgtggtgggcccgccccttgagcctgggatccagcctcagcgaggctccagaagtgtcgtctccgccggccaggcgtcagcgcctccacccaggatcacacactcctatctctgcacttctgccctccatcctgctccacgccgcctctgcttgttattccagcacattccagggtgatgcctgctgaagccacgcagccagtgcctccaccctaccccccatctgacccaccggccccttccgcacgctggaagcctgcccggcctgaagtgtcctcacctgtcgccctatcctcccctccactccagcccaccggtgactcagccctcacagctctctcctccccgagggagccggtgacttggctctgatggtgccgggacccagccccggcatgttcagaatccttgtcctttaacctgcgtccacatgtagtcgcctccacctccctgcctgaacctccaggccctgcagagcaaacactgctgcctgcaggagacccctggggccttctccgtcccctcatcctcctaaggagcctcccctgccaccgtgccagaatgtgccggaggaacagaggcccgagcctcccgtggccccagcgaggcttcctctggggaagacagtgagcccaacgccctggctctcgaggggtggacggagtccagctggcatcgaccgggtgcccctcttcaccggtgtccacagtccttgagagcaaagggttcccagaggcgacctccctcctgcacaggcatttgtcatccttaccctctaggacagctttgccatgtgcagtgaacccctggacagtgttgcgtgccactttgccggatttggactgtatacagaccgaagcatgtggcatgaagccttctgctgtcagcaagttccttgtccctcgacagtatgtgcccaggctgcccttcaaggtggcagtgatggcagttgtctcttgcacgctgcgtacagttctgtcactgactcttacacacagccctggagacgggagagtgccgggagcattctatgcaagtgtcctggcctttgcgggcgtctccacagcacaaacacgagtcagggaccacccctcttctgcttccatgattccaccaactgggtggggcagagcagtgccagggccggaggcacgccccaggtgagggcaggtggccatgccagtgagtgcactgcgttctcagttaattcatccgaaatatttctaagtttcttgattattgcagttgcatccccaagataatgccgccctcaccaagtagaaggcacatgtgctctgatggcccctcccacactggggtcagggcacagcaacttagcgaggcgacgtgggggctcagaggacctcggaccaggctcggcacatggggcttctccagccagccagggggaagcaccctgcagactgctaacttttgcaaaatagagtaaaaaagatcagctagaaaaataaaacaaaaaaggaaagatatacaaaaaaaaaaaaaaa

SEQ ID NO: 47 - RNA, 2079 nt, Homo sapiens
  source 1..2079: mol_type = mRNA
actgctgtgcagggcaggaaagctccacacacacagcccagcaaacagcagcacgctgctgaaaaaaagactcagaggagagagataaggaaggaaagtagtgatggatctcatcccaaacttggccgtggaaacctggcttctcctggctgtcagcctgatactcctctatctatatggaacccgtacacatggactttttaagaagcttggaattccagggcccacacctctgccttttttgggaaatgctttgtccttccgtaagggctattggacgtttgacatggaatgttataaaaagtatagaaaagtctggggtatttatgactgtcaacagcctatgctggctatcacagatcccgacatgatcaaaacagtgctagtgaaagaatgttattctgtcttcacaaaccggaggcctttcgggccagtgggatttatgaaaaatgccatctctatagctgaggatgaagaatggaagagaatacgatcattgctgtctccaacattcaccagcggaaaactcaaggagatggtccctatcattgcccagtatggagatgtgttggtgagaaatctgaggcgggaagcagagacaggcaagcctgtcaccttgaaacacgtctttggggcctacagcatggatgtgatcactagcacatcatttggagtgagcatcgactctctcaacaatccacaagacccctttgtggaaaacaccaagaagcttttaagatttaatccattagatccattcgttctctcaataaaagtctttccattccttaccccaattcttgaagcattaaatatcactgtgtttccaagaaaagttataagttttctaacaaaatctgtaaaacagataaaagaaggtcgcctcaaagagacacaaaagcaccgagtggatttccttcagctgatgattgactctcagaattcaaaagactctgagacccacaaagctctgtctgatctggagctcatggcccaatcaattatctttatttttgctggctatgaaaccacgagcagtgttctctccttcattatatatgaactggccactcaccctgatgtccagcagaaagtgcagaaggaaattgatacagttttacccaataaggcaccacccacctatgatactgtgctacagttggagtatcttgacatggtggtgaatgaaacactcagattattcccagttgctatgagacttgagagggtctgcaaaaaagatgttgaaatcaatgggatgtttattcccaaaggggtggtggtgatgattccaagctatgttcttcatcatgacccaaagtactggacagagcctgagaagttcctccctgaaaggttcagtaaaaagaacaaggacaacatagatccttacatatacacaccctttggaagtggacccagaaactgcattggcatgaggtttgctctcgtgaacatgaaacttgctctagtcagagtccttcagaacttctccttcaaaccttgtaaagaaacacagatccccctgaaattacgctttggaggacttcttctaacagaaaaacccattgttctaaaggctgagtcaagggatgagaccgtaagtggagcctgatttccctaaggacttctggtttgctctttaagaaagctgtgccccagaacaccagagacctcaaattactttacaaatagaaccctgaaatgaagacgggcttcatccaatgtgctgcataaataatcagggattctgtacgtgcattgtgctctctcatggtctgtatagagtgttatacttggtaatatagaggagatgaccaaatcagtgctggggaagtagatttggcttctctgcttctcataggactatctccaccacccccagttagcaccattaactcctcctgagctctgataacataattaacatttctcaataatttcaaccacaatcattaataaaaataggaattattttgatggctctaacagtgacatttatatcatgtgttatatctgtagtattctatagtaagctttatattaagcaaatcaataaaaacctctttacaaaagtat

SEQ ID NO: 48 - RNA, 6348 nt, Homo sapiens
  source 1..6348: mol_type = mRNA
aattaattttacttaggataagtgttgttattattgtttttattgttgttctgttagttactcaaaacttcattctaattgtgccctgagtttgttaaaataccatactgtatttttgtgtaacatgtaaataggcattaatttttgagaaatagaaatgtttatccttaatgtatttttaatttgctaacattgattttttattttctttcctgaaatagcttatttcctaaaatgaaagaatttattctcagatgaataatttttatatcagctattcttatcagagcaataaacaaataccaatgatgcgctcagccaacaattcattacactctctgaagagtaactggacaaggagaaaaacatagggaaaaaaccaacagaatttgttggcatgttctacacacagaccatggcttttcagaagccaagctgaatcaaaacagttttaaaagaggcaaccatttgtagaggagtccttgaaggattcttcattgttttcttggacaaaaagagaccagtggatccaagtgcttcaaatacttctctcttattttcttaactctattgctctgcaatatttactttaccctgttaatgaacaggacaaaatggttaaaaaagagataagcgtgcgtcaacaaattcaggctcttctgtacaagaattttcttaaaaaatggagaataaaaagagagtttattggaatggacaataacattgtttctagggctatatttgtgcatcttttcggaacacttcagagctacccgttttcctgaacaacctcctaaagtcctgggaagcgtggatcagtttaatgactctggcctggtagtggcatatacaccagtcagtaacataacacaaaggataatgaataagatggccttggcttcctttatgaaaggaagaacagtcattgggacaccagatgaagagaccatggatatagaacttccaaaaaaataccatgaaatggtgggagttatatttagtgatactttctcatatcgcctgaagtttaattggggatatagaatcccagttataaaggagcactctgaatacacagaacactgttgggccatgcatggtgaaattttttgttacttggcaaagtactggctaaaagggtttgtagcttttcaagctgcaattaatgctgcaattatagaagtcacaacaaatcattctgtaatggaggagttgacatcagttattggaataaatatgaagataccacctttcatttctaagggagaaattatgaatgaatggtttcattttacttgcttagtttctttctcttcttttatatactttgcatcattaaatgttgcaagggaaagaggaaaatttaagaaactgatgacagtgatgggtctccgagagtcagcattctggctctcctggggattgacatacatttgcttcatcttcattatgtccatttttatggctctggtcataacatcaatcccaattgtatttcatactggcttcatggtgatattcacactctatagcttatatggcctttctttgatagcattggctttcctcatgagtgttttaataaggaaacctatgctcgctggtttggctggatttctcttcactgtattttggggatgtctgggattcactgtgttatatagacaacttcctttatctttgggatgggtattaagtcttcttagcccttttgccttcactgctggaatggcccagattacacacctggataattacttaagtggtgttatttttcctgatccctctggggattcatacaaaatgatagccacttttttcattttggcatttgatactcttttctatttgatattcacattatattttgagcgagttttacctgataaagatggccatggggattctccattatttttccttaagtcctcattttggtccaaacatcaaaatactcatcatgaaatctttgagaatgaaataaatcctgagcattcctctgatgattcttttgaaccggtgtctccagaattccatggaaaagaagccataagaatcagaaatgttataaaagaatataatggaaagactggaaaagtagaagcattgcaaggcatattttttgacatatatgaaggacagatcactgcaatacttgggcataatggagctggtaaatcaacactgctaaacattcttagtggattgtctgtttctacagaaggatcagccactatttataatactcaactctctgaaataactgacatggaagaaattagaaagaatattggattttgtccacagttcaattttcaatttgacttcctcactgtgagagaaaacctcagggtatttgctaaaataaaagggattcagccaaaggaagtggaacaagaggtaaaaagaattataatggaattagacatgcaaagcattcaagacattattgctaaaaaattaagtggtgggcagaagagaaaactaacactagggattgccatcttaggagatcctcaggttttgctgctagatgaaccaactgctggattggatcccttttcaagacaccgagtgtggagcctcctgaaggagcataaagtagaccgacttatcctcttcagtacccaattcatggatgaggctgacatcttggctgataggaaagtatttctgtctaatgggaagttgaaatgtgcaggatcatctttgtttctgaagcgaaagtggggtattggatatcatttaagtttacacaggaatgaaatgtgtgacacagaaaaaatcacatcccttattaagcagcacattcctgatgccaagttaacaacagaaagtgaagaaaaacttgtatatagtttgcctttggaaaaaacgaacaaatttccagatctttacagtgaccttgataagtgttctgaccagggcataaggaattatgctgtttcagtgacatctctgaatgaagtattcttgaacctagaaggaaaatcagcaattgatgaaccagattttgacattgggaaacaagagaaaatacatgtgacaagaaatactggagatgagtctgaaatggaacaggttctttgttctcttcctgaaacaagaaaggctgtcagtagtgcagctctctggagacgacaaatctatgcagtggcaacacttcgcttcttaaagttaaggcgtgaaaggagagctcttttgtgtttgttactagtacttggaattgcttttatccccatcattctagagaagataatgtataaagtaactcgtgaaactcattgttgggagttttcacccagtatgtatttcctttctctggaacaaatcccgaagacgcctcttaccagcctgttaatcgttaataatacaggatcaaatattgaagacctcgtgcattcactgaagtgtcaggatatagttttggaaatagatgactttagaaacagaaatggctcagatgatccctcctacaatggagccatcatagtgtctggtgaccagaaggattacagattttctgttgcgtgtaataccaagaaattgaattgttttcctgttcttatgggaattgttagcaatgcccttatgggaatttttaacttcacggagcttattcaaatggagagcacttcattttctcgtgatgacatagtgctggatcttggttttatagatgggtccatatttttgttgttgatcacaaactgcgtttctccttttatcggcatgagcagcatcagcgattataaaaaaaatgttcaatcccagttatggatttcaggcctctggccttcagcatactggtgtggacaggctctggtggacattccattatacttcttgattctcttttcaatacatttaatttactacttcatatttctgggattccagctttcatgggaactcatgtttgttttggtggtatgcataattggttgtgcagtttctcttatattcctcacatatgtgctttcattcatctttcgcaagtggagaaaaaataatggcttttggtcttttggcttttttattatcttaatatgtgtatccacaattatggtatcaactcaatatgaaaaactcaacttaattttgtgcatgattttcataccttccttcactttgctggggtatgtcatgttattgatccagctcgactttatgagaaacttggacagtctggacaatagaataaatgaagtcaataaaaccattcttttaacaaccttaataccataccttcagagtgttattttcctttttgtcataaggtgtctggaaatgaagtatggaaatgaaataatgaataaagacccagttttcagaatctctccacggagtagagaaactcatcccaatccggaagagcccgaagaagaagatgaagatgttcaagctgaaagagtccaagcagcaaatgcactcactgctccaaacttggaggaggaaccagtcataactgcaagctgtttacacaaggaatattatgagacaaagaaaagttgcttttcaacaagaaagaagaaaatagccatcagaaatgtttccttttgtgttaaaaaaggtgaagttttgggattactaggacacaatggagctggtaaaagtacttccattaaaatgataactgggtgcacaaagccaactgcaggagtggtggtgttacaaggcagcagagcatcagtaaggcaacagcatgacaacagcctcaagttcttggggtactgccctcaggagaactcactgtggcccaagcttacaatgaaagagcacttggagttgtatgcagctgtgaaaggactgggcaaagaagatgctgctctcagtatttcacgattggtggaagctcttaagctccaggaacaacttaaggctcctgtgaaaactctatcagagggaataaagagaaagctgtgctttgtgctgagcatcctggggaacccatcagtggtgcttctagatgagccgttcaccgggatggaccccgaggggcagcagcaaatgtggcagatacttcaggctaccgttaaaaacaaggagaggggcaccctcttgaccacccattacatgtcagaggctgaggctgtgtgtgaccgtatggccatgatggtgtcaggaacgctaaggtgtattggttccattcaacatctgaaaaacaagtttggtagagattatttactagaaataaaaatgaaagaacctacccaggtggaagctctccacacagagattttgaagcttttcccacaggctgcttggcaggaaagatattcctctttaatggcgtataagttacctgtggaggatgtccaccctctatctcgggcctttttcaagttagaggcgatgaaacagaccttcaacctggaggaatacagcctctctcaggctaccttggagcaggtattcttagaactctgtaaagagcaggagctgggaaatgttgatgataaaattgatacaacagttgaatggaaacttctcccacaggaagacccttaaaatgaagaacctcctaacattcaattttaggtcctactacattgttagtttccataattctacaagaatgtttccttttacttcagttaacaaaagaaaacatttaataaacattcaataatgattacagttttcatttttaaaaatttaggatgaaggaaacaaggaaatatagggaaaagtagtagacaaaattaacaaaatcagacatgttattcatccccaacatgggtctattttgtgcttaaaaataatttaaaaatcatacaatattaggttggttatcggttattatcaataaagctaacactgagaacattttacaaataaaaatatgaggtttttagcctgaacttcaaatgtatcagctatttttaaacattatttactcggattctaatttaatgtgacattgactataagaaggtctgataaactgatgaaatggcacagcataacatttaattataatgacattctgattataaaataaatgcatgtgaattttagtacatattgaagttatatggaagaagatagccataatctgtaagaaagtaccgcagttaatattttctttagccaacttatattcaatgtattttttatggatcctttttcaaaggtagtatcagtaggcatagtcattttctgtatcttttcacctcacagttcatgaacatttcccatgtcattatagcacttttgtgttatataattgctgcattaatgttatagctgtattgatgtaataccagagttgttacgttcgggttatatgcaagtttatttcttagaaataaaactttgatggacttttaaaaaaaaaaaaaaaaa

SEQ ID NO: 49 - RNA, 1892 nt, Homo sapiens
  source 1..1892: mol_type = mRNA
aaaccggtgccaacgtgcgcggacgccgccgccgccgccgccgctggagtccgccgggcagagccggccgcggagcccggagcaggcggagggaagtgcccctagaaccagctcagccagcggcgcttgcacagagcggccggacgaagagcagcgagaggaggaggggagagcggctcgtccacgcgccctgcgccgccgccggcccgggaaggcagcgaggagccggcgcctcccgcgccccgcggtcgccctggagtaatttcggatgcccagccgcggccgccttccccagtagacccgggagaggagttgcggccaacttgtgtgcctttcttccgccccggtgggagccggcgctgcgcgaagggctctcccggcggctcatgctgccggccctgcgcctgcccagcctcgggtgagccgcctccggagagacgggggagcgcggcggcgccgcgggctcggcgtgctctcctccggggacgcgggacgaagcagcagccccgggcgcgcgccagaggcatggagcgctgccccagcctaggggtcaccctctacgccctggtggtggtcctggggctgcgggcgacaccggccggcggccagcactatctccacatccgcccggcacccagcgacaacctgcccctggtggacctcatcgaacacccagaccctatctttgaccccaaggaaaaggatctgaacgagacgctgctgcgctcgctgctcgggggccactacgacccaggcttcatggccacctcgccccccgaggaccggcccggcgggggcgggggtgcagctgggggcgcggaggacctggcggagctggaccagctgctgcggcagcggccgtcgggggccatgccgagcgagatcaaagggctagagttctccgagggcttggcccagggcaagaagcagcgcctaagcaagaagctgcggaggaagttacagatgtggctgtggtcgcagacattctgccccgtgctgtacgcgtggaacgacctgggcagccgcttttggccgcgctacgtgaaggtgggcagctgcttcagtaagcgctcgtgctccgtgcccgagggcatggtgtgcaagccgtccaagtccgtgcacctcacggtgctgcggtggcgctgtcagcggcgcgggggccagcgctgcggctggattcccatccagtaccccatcatttccgagtgcaagtgctcgtgctagaactcgggggccccctgcccgcacccggacacttgatcgatccccaccgacgccccctgcaccgcctccaaccagttccaccaccctctagcgagggttttcaatgaacttttttttttttttttttttttttttctgggctacagagacctagctttctggttcctgtaatgcactgtttaactgtgtaggaatgtatatgtgtgtgtatatacggtcccagttttaatttacttattaaaaggtcagtattatacgttaaaagttaccggcttctactgtatttttaaaaaaaagtaagcaaaagaaaaaaaaaagaacagagaaaagagagacttattctggttgttgctaataatgttaacctgctatttatattccagtgcccttcgcatggcgaagcaggggggaaaagttatttttttcttgaagtacaaagagacgggggaacttttgtagaggactttttaaaagctattttccattcttcggaaagtgttttggttttccttggacctcgaagaagctatagagttcaatgttattttacagttattgtaaatatagagaacaaatggaatgactaatcattgtaaattaagagtatctgctatttattctttataatatcccgtgtagtaaatgagaaagaagtgcagagcaggatt

SEQ ID NO: 50 - RNA, 3227 nt, Homo sapiens
  source 1..3227: mol_type = mRNA
cccactctgcagttgtctcccgagcgctggctgcgccgcccgagccgctgggccggggaagcactggccgttcgctcccgggccggctccgccaggcgctcgcaggcatgcagcccgggagcaggaggcgctccccgggccgctgctgagccggccggggcggcggggaccagcgccagcggagcccctcccaccttgccccggggcagacgagcggcgccccgacaccccctcttctcccgcagccccgccagcgccaccccccgcgggccgcaggggctcatgcagccgccaagggagaggctagtggtaacaggccgagctggatggatgggtatggggagaggggcaggacgttcagccctgggattctggccgaccctcgccttccttctctgcagcttccccgcagccacctccccgtgcaagatcctcaagtgcaactctgagttctggagcgccacgtcgggcagccacgccccagcctcagacgacacccccgagttctgtgcagccttgcgcagctacgccctgtgcacgcggcggacggcccgcacctgccggggtgacctggcctaccactcggccgtccatggcatagaggacctcatgagccagcacaactgctccaaggatggccccacctcgcagccacgcctgcgcacgctcccaccggccggagacagccaggagcgctcggacagccccgagatctgccattacgagaagagctttcacaagcactcggccacccccaactacacgcactgtggcctcttcggggacccacacctcaggactttcaccgaccgcttccagacctgcaaggtgcagggcgcctggccgctcatcgacaataattacctgaacgtgcaggtcaccaacacgcctgtgctgcccggctcagcggccactgccaccagcaagctcaccatcatcttcaagaacttccaggagtgtgtggaccagaaggtgtaccaggctgagatggacgagctcccggccgccttcgtggatggctctaagaacggtggggacaagcacggggccaacagcctgaagatcactgagaaggtgtcaggccagcacgtggagatccaggccaagtacatcggcaccaccatcgtggtgcgccaggtgggccgctacctgacctttgccgtccgcatgccagaggaagtggtcaatgctgtggaggactgggacagccagggtctctacctctgcctgcggggctgccccctcaaccagcagatcgacttccaggccttccacaccaatgctgagggcaccggtgcccgcaggctggcagccgccagccctgcacccacagcccccgagaccttcccatacgagacagccgtggccaagtgcaaggagaagctgccggtggaggacctgtactaccaggcctgcgtcttcgacctcctcaccacgggcgacgtgaacttcacactggccgcctactacgcgttggaggatgtcaagatgctccactccaacaaagacaaactgcacctgtatgagaggactcgggacctgccaggcagggcggctgcggggctgcccctggccccccggcccctcctgggcgccctcgtcccgctcctggccctgctccctgtgttctgctagacgcgtagatgtggagggaggcgcgggctccgtcctctcggcttccccatgtgtgggctgggaccgcccacggggtgcagatctcctggcgtgtccaccatggccccgcagaacgccagggaccgcctgctgccaagggctcaggcacggacccctccccttctagtgcacgtgacaaggttgtggtgactggtgccatgatgtttgacagtagagctgtgtgagagggagagcagctcccctcgccccgcccctgcagtgtgaatgtgtgaaacatcccctcaggctgaagccccccacccccaccagagacacactgggaaccgtcagagtcagctccttccccctcgcaatgcactgaaaggcccggccgactgctgctcgccgatccgtggggccccctgtgcccgccacacgcacgcacacactcttacacgagagcacactcgatccccctaggccagcggggacaccccagccacacagggaggcatccttggggcttggccccaggcagggcaaccccggggcgctgcttggcaccttagcagactgctggaaccttttggccagtaggtcgtgcccgcctggtgccttctggcctgtggcctccctgcccatgttcacctggctgctgtgggtaccagtgcaggtcccggttttcaggcacctgctcagctgcccgtctctggcctgggcccctgccccttccaccctgtgcttagaaagtcgaagtgcttggttctaaatgtctaaacagagaagagatccttgacttctgttcctctctctcctgcagatgcaagagctcctgggcaggggtgcctgggccccagggtgtggcaggagacccagtggatggggccagctggcctgccctgatcctctgcttcctcctcacaaccccaagagcccccagcccggtccatccacgtctggagtctggggagaggagcagggtcttaggactctcagctctgagcatccctggcagggtcttcaacctctaatctcttcccttaagccctgtggccacacagccaggagagacttgccgctggctcccgcctcatttcagcccagggtgctcatccaggggcccagaacagtcccacctgtgctgctgtgcccacagcacaaagccaggcttcactcccaaaagtgcagccaggccctggagggtgatcctgccagcagccctacagctccacaccctacccacccatcggcagcccctctgctgttccccagggacctctcatacactggccaggaggctgcagaacgtgtgtctccccctccctccaagaggtcctgctccctctgccagaaccgtgtgtgggcgggtgggagggcgctcggggcccggcccctccctctccctgctggttttagttggtccctatgttggaagtaaaaagtgaagcactttattttggttgtgtttgctcacgttctgcttggaagtggggacccctcactgcgtccacgtgtctgcgacctgtgtggagtgtcaccgcgtgtacatactgtaaattatttattaatggctaaatgcaagtaaagtttggtttttttgttattttctttta

SEQ ID NO: 51 - RNA, 3200 nt, Homo sapiens
  source 1..3200: mol_type = mRNA
ccatactgaagccctcccaagtgcagccttgcgggcctcgcagagagcgccaaaccatccggccctgcgcgccacgcggaccggcatagccatctctctggagtggcagctgtgccaaggtggcaaggttaggcccgcaggccctcagcacgggggcgcagccggggcaagtcttcaggaagcagcgaggtgcttgaagggggtgctggaggcaggccaaacccagtcaggatgggtggcctggggccacgacgggcgggaacctcgagggagaggctagtggtaacaggccgagctggatggatgggtatggggagaggggcaggacgttcagccctgggattctggccgaccctcgccttccttctctgcagcttccccgcagccacctccccgtgcaagatcctcaagtgcaactctgagttctggagcgccacgtcgggcagccacgccccagcctcagacgacacccccgagttctgtgcagccttgcgcagctacgccctgtgcacgcggcggacggcccgcacctgccggggtgacctggcctaccactcggccgtccatggcatagaggacctcatgagccagcacaactgctccaaggatggccccacctcgcagccacgcctgcgcacgctcccaccggccggagacagccaggagcgctcggacagccccgagatctgccattacgagaagagctttcacaagcactcggccacccccaactacacgcactgtggcctcttcggggacccacacctcaggactttcaccgaccgcttccagacctgcaaggtgcagggcgcctggccgctcatcgacaataattacctgaacgtgcaggtcaccaacacgcctgtgctgcccggctcagcggccactgccaccagcaagctcaccatcatcttcaagaacttccaggagtgtgtggaccagaaggtgtaccaggctgagatggacgagctcccggccgccttcgtggatggctctaagaacggtggggacaagcacggggccaacagcctgaagatcactgagaaggtgtcaggccagcacgtggagatccaggccaagtacatcggcaccaccatcgtggtgcgccaggtgggccgctacctgacctttgccgtccgcatgccagaggaagtggtcaatgctgtggaggactgggacagccagggtctctacctctgcctgcggggctgccccctcaaccagcagatcgacttccaggccttccacaccaatgctgagggcaccggtgcccgcaggctggcagccgccagccctgcacccacagcccccgagaccttcccatacgagacagccgtggccaagtgcaaggagaagctgccggtggaggacctgtactaccaggcctgcgtcttcgacctcctcaccacgggcgacgtgaacttcacactggccgcctactacgcgttggaggatgtcaagatgctccactccaacaaagacaaactgcacctgtatgagaggactcgggacctgccaggcagggcggctgcggggctgcccctggccccccggcccctcctgggcgccctcgtcccgctcctggccctgctccctgtgttctgctagacgcgtagatgtggagggaggcgcgggctccgtcctctcggcttccccatgtgtgggctgggaccgcccacggggtgcagatctcctggcgtgtccaccatggccccgcagaacgccagggaccgcctgctgccaagggctcaggcacggacccctccccttctagtgcacgtgacaaggttgtggtgactggtgccatgatgtttgacagtagagctgtgtgagagggagagcagctcccctcgccccgcccctgcagtgtgaatgtgtgaaacatcccctcaggctgaagccccccacccccaccagagacacactgggaaccgtcagagtcagctccttccccctcgcaatgcactgaaaggcccggccgactgctgctcgccgatccgtggggccccctgtgcccgccacacgcacgcacacactcttacacgagagcacactcgatccccctaggccagcggggacaccccagccacacagggaggcatccttggggcttggccccaggcagggcaaccccggggcgctgcttggcaccttagcagactgctggaaccttttggccagtaggtcgtgcccgcctggtgccttctggcctgtggcctccctgcccatgttcacctggctgctgtgggtaccagtgcaggtcccggttttcaggcacctgctcagctgcccgtctctggcctgggcccctgccccttccaccctgtgcttagaaagtcgaagtgcttggttctaaatgtctaaacagagaagagatccttgacttctgttcctctctctcctgcagatgcaagagctcctgggcaggggtgcctgggccccagggtgtggcaggagacccagtggatggggccagctggcctgccctgatcctctgcttcctcctcacaaccccaagagcccccagcccggtccatccacgtctggagtctggggagaggagcagggtcttaggactctcagctctgagcatccctggcagggtcttcaacctctaatctcttcccttaagccctgtggccacacagccaggagagacttgccgctggctcccgcctcatttcagcccagggtgctcatccaggggcccagaacagtcccacctgtgctgctgtgcccacagcacaaagccaggcttcactcccaaaagtgcagccaggccctggagggtgatcctgccagcagccctacagctccacaccctacccacccatcggcagcccctctgctgttccccagggacctctcatacactggccaggaggctgcagaacgtgtgtctccccctccctccaagaggtcctgctccctctgccagaaccgtgtgtgggcgggtgggagggcgctcggggcccggcccctccctctccctgctggttttagttggtccctatgttggaagtaaaaagtgaagcactttattttggttgtgtttgctcacgttctgcttggaagtggggacccctcactgcgtccacgtgtctgcgacctgtgtggagtgtcaccgcgtgtacatactgtaaattatttattaatggctaaatgcaagtaaagtttggtttttttgttattttctttta

SEQ ID NO: 52 - RNA, 590 nt, Homo sapiens
  source 1..590: mol_type = mRNA
atcccactgggaagcttcaacatagctgtggaagtctgcagtctacaggagcctactatagacattctacaaccaaccagaatcatggaacagccaacttccagcaccaatggggagaagacgaagagcccctgtgaatccaataacaaaaaaaatgatgagatgcaagaggtaccaaacagagtcttagcccccgaacagagtttgaagaacacaaaaacatcagaatatccaataatatttgtgtattacctcaggaagggtaagaaaataaattcaaatcaactggagaatgaacagtcccaagagaactccatcaatccaatccaaaaggaggaggacgaaggcgtagacttatctgaaggatcttcaaatgaggatgaagacctaggcccatgtgaaggaccttcaaaggaggacaaagatctagactcatctgaaggatcctcacaggaggatgaagacctaggcttatctgaaggatcttcacaggacagtggggaggattagtcacacatggagaaaccaaattggacaaatcatcaccactgatggcgatgattacaataaaatcaagtttaaggagctga

SEQ ID NO: 53 - RNA, 1408 nt, Homo sapiens
  source 1..1408: mol_type = mRNA
caccttcctcccatcttgccttctccctcgagttgggacccgggaagaaccatgaagtggctgctgctgctgggtctggtggcgctctctgagtgcatcatgtacaaggtccccctcatcagaaagaagtccttgaggcgcaccctgtccgagcgtggcctgctgaaggacttcctgaagaagcacaacctcaacccagccagaaagtacttcccccagtgggaggctcccaccctggtagatgaacagcccctggagaactacctggatatggagtacttcggcactatcggcatcggaactcctgcccaggatttcaccgtcgtctttgacaccggctcctccaacctgtgggtgccctcagtctactgctccagtcttgcctgcaccaaccacaaccgcttcaaccctgaggattcttccacctaccagtccaccagcgagacagtctccatcacctacggcaccggcagcatgacaggcatcctcggatacgacactgtccaggttggaggcatctctgacaccaatcagatcttcggcctgagcgagacggaacctggctccttcctgtattatgctcccttcgatggcatcctggggctggcctaccccagcatttcctcctccggggccacacccgtctttgacaacatctggaaccagggcctggtttctcaggacctcttctctgtctacctcagcgccgatgacaagagtggcagcgtggtgatctttggtggcattgactcttcttactacactggaagtctgaactgggtgcctgttaccgtcgagggttactggcagatcaccgtggacagcatcaccatgaacggagagaccatcgcctgtgctgagggctgccaggccattgttgacaccggcacctctctgctgaccggcccaaccagccccattgccaacatccagagcgacatcggagccagcgagaactcagatggcgacatggtggtcagctgctcagccatcagcagcctgcccgacatcgtcttcaccatcaatggagtccagtaccccgtgccacccagtgcctacatcctgcagagcgaggggagctgcatcagtggcttccagggcatgaacgtccccaccgaatctggagagctttggatcctgggtgatgtcttcatccgccagtactttaccgtcttcgacagggcaaacaaccaggtcggcctggcccctgtggcttaagcctaagtctcttcagccacctcccaggaagatctggcctccgtcctatgcccactttagatgtatctaattctcctgactgttcttcccaggggagtgtgaaggtcttggccctgttccctgtcctaccaataacgtagaataaaaacataacccactgaaaaaaaaaaaaaaaaaaaaaaaaaaaaaa

SEQ ID NO: 54 - RNA, 5073 nt, Homo sapiens
  source 1..5073: mol_type = mRNA
aggctggccctggctgcctgcggcgcgggcgcctccctcgcagccgctgctgccgacgccgctgcgctcccgcctcctgtcggttcccgggttcccgctgcggctgcggttctggcagccgagcccccgcggtgctgcagcccagctttagcgcgcagaccgacccgcgccccttcttcgccgccggcagcctctaatccacgcggcgcgttgcggcaggtgccctgggcgtactgaggcgcggtggcctgagcccggccgccatcgatgaccccggtcgcggacttgcttcaggctggccaccccccgtcttgtttcatcatctgtgttgagtaaccatggggaggaagctggacctgtctggtttgactgatgatgaaacagagcatgttcttcaggtggttcaaagagacttcaatcttcgcaaaaaagaagaagaacgactaagtgagctgaagcagaagctggatgaggaaggcagcaagtgcagcatcctctcgaagcaccagcagtttgtggagcactgctgcatgcgctgctgctcgcccttcaccttcctcgtcaacaccaagcgccagtgtggagattgcaaattcaatgtctgcaagagctgctgctcctaccagaagcacgaaaaggcctgggtctgctgcgtctgccagcaagcgaggcttctgagggcccaatctctggaatggttctacaataatgtgaagagccgcttcaagcgctttggcagtgccaaggttctgaagaacctgtacaggaagcaccggctggagagtggcgcgtgcttcgacattctaggaggaagcctttttgagtcaaacctggagaatgaaggaagcatttctggcagtgattcaacattttataggcagtcagaaggacatagtgtgatggacaccttggctgtggccctacgggtggctgaagaggccattgaggaagcaatttccaaagcagaggcatatggggacagcctggacaagcaaaatgaggccagttacctgcgggaccacaaggaggagctaactgaggaactggccacgacaatcctgcagaagattatacgaaaacagaagagcaaaagtgagcagcaagtggaagaagagccaggatggccacatccccagagttgcagcacaaaggtggcagatgaggggacctcagcatcccctggaggctaccgtgctcccgctgccctctggaggtcccagtctgccttctcaatcactggagaagaagccctgaagacccctccagtggaggctccatcgaggcagccaagggaccaaggccaacacccgagagcagagtctgctctgcccagctggaagagtgtggacaggctggatgaaacaaacctggccccagttttgcagagccccgacgggaactgggtggccctgaaggatggcgctccaccccccacccgactactggccaaacctaagagcgggacgtttcaggccctggaggtggcctccagtgtggcatctgcctacgatgagatgggctccgatagcgaggaagactttgactggagtgaggccttgagcaagctgtgtcccaggtcccgggccctgcccaggaacccccagcctcagcccacacaggcccagagctctgaccaaggccccatagctgcctccccatcctctgcactctcccccaaccctgaggccatgtgctctgactcggagacctcctccgcaggctcttcccgagaagttgggcaccaggccagactgtcctggttgcagaggaaggcccccaggaaccctgcagctgagaagatgcgcttgcatggagagctggacgtgaacttcaacccccagttggccagcagggagacctcggacagcagcgagccggaggaggccccccacaccacagaccggcgggccaggaggtggagaagagcccgactgggctcagaagagccaagcaaagaaccatcttcccccagcgcccagctccgggatctagacacacatcaggtgtcggatgatttatcagagacagacatcagcaatgaggctcgggatccccagactctcacagacaccacagaggagaaacggagaaacaggctgtacgagttagcaatgaaaatgagtgaaaaggagacttcttcaggggaggatcaggagtctgagcccaagacagaatctgagaaccagaaggaaagtctgtcctctgaagacaacagccagagtgtccaggaagagctgaagaagaagttttctgctgtttctctctgcaacatctccacagaagtcctgaaagtcatcaatgccacagaggagttgatagcaggatctacagggccctgggagtccccacaagtccctcctgacagacagaaggggatgtttcctcgtgggacagaccaagtgagactggatgagcagctgacttccctggaagaaaatgtatacctggcagcaggcactgtgtatggactggagacccagctgactgagctagaagatgccgcccgctgcatccacagtggcactgatgagacccatctggcggatctggaggaccaggtggccacggctgcagcccaagtccaccatgctgaactccagatttcagatattgagagccggatttcagccctgaccattgcaggattaaacatagcaccatgtgtgcgcttcacaagaagacgggatcagaagcaaaggacccaggtacaaaccatagatacatcaaggcagcaaaggaggaaactgcctgctccaccggtgaaagctgaaaaaattgagacatcttcagtgactaccattaaaacatttaaccacaacttcattctccaaggctcctcaacaaacaggactaaggaaaggaaaggcaccaccaaggatttgatggagcctgctctggagtcagctgtgatgtactgacaccatggaattccactgccagtgacccactgcctccggccgtacacgacagtgccttgacccaacagccatcgagtactgtatgtatttccacctgaggagaaggcctggggaggccacagtgcaccattgcacagggctgtcctgatacctcatccagaaagccgtctcagacttcagcactgcggtcttgcccactctctgccttaggctcccaggggaatccaagacagaaaatgaagacactggcttccaacagcagcgctccatgtttaagatacatattttccctgtttgctttgctactgtatgttgactttaagatctttttttaaatacatttgattcagctagtattccatgtcaacaatttgtccaaaggaaaactgctggagggaggtggagggaggaaggtgggaattattatttaatacatcattaatgcttattaatctctcacaagcatctttgtcttgcaaatcctaagggaaaagcaagtccctgcagtgagcactagggacagtctaatttggggattgctcaaccatcaagactgcaggtctcccttcagccacctccttcctgctaaaagcttagcctaccacactaccagtcattcccatcgctttgcaatcacaagccacaggatgagaagttctgactcactcatgccatgcccagggctatctgaaacaatgtctcattaagaatttagggttcttccatgggcttactgacagttgcccagatctgaaggggaaagggtcttgagaaagaccatcactggctcaactttagggcactgtccagagtcaacatgatgtggtttagcagtgatcacatctaaacaaagtttaggtaaatgaattatcgcagagaaaaaccacatgagaaaatttttgtactccaaatttacttcccaataaatattcagcaaagtagtaaaatgaccttaaagataaaaatgattagggaatagccttagaaaatttataggtataaaaaattcaaggacaaactgtgcatttaatggacacaagaattgactctaactccatgtctgtggtttctttgaacccatatcaaatgtatgactatttagagtgtttataagagataatggaactgaactttcactcaattaattgggcattaacaaccttcttttatgtttgttcctgatatagtctgaatcttaggaagaaggtaaaagaaaggaggcaagagaatagttatgatgaatatgtgttaagtgcctgctctgaaggaggcaatgttcttctcatttgaatccttatggcaaccttattcaataggttttcccatatttcagatttaataactgaaggccagagagattaatttgccaaagccacacctttatgctaattatgattggaatgcatcacaaaagcctaactctgttgttttcaacctctacgttattttgctgctatgtgcatttccagatctgattttctgctaacttgtgtgctatgatccactcctgatgggggtctacattaatcttccagtactccttgctgatgctgtgttatgtgtcatctaacagaaatgactcctttgaaataagtaaatctttggctttttgttctgttggtgtgattcaaagcaaaacaaacaaacaaaaacaaattttaagaacacaacaaaaaagatttgacttccgaatagaatgttttctttaagaggcatgaaaagcaactattgttgtgttacagtgttaaaaatattcagttttctttgacaaaaatgtgtactgtgtaagccttgcaaacaaaaaacaacaaaaaagaagcagcagcagcagcctgctgtgtggcatctgaacttttataaaggtttccttgtgccaaataagtgcaaagatttaatttactattaaaaaccataagcatatgttatagttccagaagaattattttgtcatcaagtgattttgatctttagtgtcaatatttatatttagattaatttttataaatgaaaatattttaatggtttaagaaaatgaggacaacaggataatatctttgatgacttctgaaagttatgcttcccttcatgttatatgcacattgccaagaattactgtcaagagaaatgataagtaaaagtcatttatgaaaataaa

SEQ ID NO: 55 - RNA, 4857 nt, Homo sapiens
  source 1..4857: mol_type = mRNA
aacccggcggttccgcagctgtgtttgggagacttgggggcgcattagcctggacagctccctgatcggtcttgtttcatcatctgtgttgagtaaccatggggaggaagctggacctgtctggtttgactgatgatgaaacagagcatgttcttcaggtggttcaaagagacttcaatcttcgcaaaaaagaagaagaacgactaagtgagctgaagcagaagctggatgaggaaggcagcaagtgcagcatcctctcgaagcaccagcagtttgtggagcactgctgcatgcgctgctgctcgcccttcaccttcctcgtcaacaccaagcgccagtgtggagattgcaaattcaatgtctgcaagagctgctgctcctaccagaagcacgaaaaggcctgggtctgctgcgtctgccagcaagcgaggcttctgagggcccaatctctggaatggttctacaataatgtgaagagccgcttcaagcgctttggcagtgccaaggttctgaagaacctgtacaggaagcaccggctggagagtggcgcgtgcttcgacattctaggaggaagcctttttgagtcaaacctggagaatgaaggaagcatttctggcagtgattcaacattttataggcagtcagaaggacatagtgtgatggacaccttggctgtggccctacgggtggctgaagaggccattgaggaagcaatttccaaagcagaggcatatggggacagcctggacaagcaaaatgaggccagttacctgcgggaccacaaggaggagctaactgaggaactggccacgacaatcctgcagaagattatacgaaaacagaagagcaaaagtgagcagcaagtggaagaagagccaggatggccacatccccagagttgcagcacaaaggtggcagatgaggggacctcagcatcccctggaggctaccgtgctcccgctgccctctggaggtcccagtctgccttctcaatcactggagaagaagccctgaagacccctccagtggaggctccatcgaggcagccaagggaccaaggccaacacccgagagcagagtctgctctgcccagctggaagagtgtggacaggctggatgaaacaaacctggccccagttttgcagagccccgacgggaactgggtggccctgaaggatggcgctccaccccccacccgactactggccaaacctaagagcgggacgtttcaggccctggaggtggcctccagtgtggcatctgcctacgatgagatgggctccgatagcgaggaagactttgactggagtgaggccttgagcaagctgtgtcccaggtcccgggccctgcccaggaacccccagcctcagcccacacaggcccagagctctgaccaaggccccatagctgcctccccatcctctgcactctcccccaaccctgaggccatgtgctctgactcggagacctcctccgcaggctcttcccgagaagttgggcaccaggccagactgtcctggttgcagaggaaggcccccaggaaccctgcagctgagaagatgcgcttgcatggagagctggacgtgaacttcaacccccagttggccagcagggagacctcggacagcagcgagccggaggaggccccccacaccacagaccggcgggccaggaggtggagaagagcccgactgggctcagaagagccaagcaaagaaccatcttcccccagcgcccagctccgggatctagacacacatcaggtgtcggatgatttatcagagacagacatcagcaatgaggctcgggatccccagactctcacagacaccacagaggagaaacggagaaacaggctgtacgagttagcaatgaaaatgagtgaaaaggagacttcttcaggggaggatcaggagtctgagcccaagacagaatctgagaaccagaaggaaagtctgtcctctgaagacaacagccagagtgtccaggaagagctgaagaagaagttttctgctgtttctctctgcaacatctccacagaagtcctgaaagtcatcaatgccacagaggagttgatagcaggatctacagggccctgggagtccccacaagtccctcctgacagacagaaggggatgtttcctcgtgggacagaccaagtgagactggatgagcagctgacttccctggaagaaaatgtatacctggcagcaggcactgtgtatggactggagacccagctgactgagctagaagatgccgcccgctgcatccacagtggcactgatgagacccatctggcggatctggaggaccaggtggccacggctgcagcccaagtccaccatgctgaactccagatttcagatattgagagccggatttcagccctgaccattgcaggattaaacatagcaccatgtgtgcgcttcacaagaagacgggatcagaagcaaaggacccaggtacaaaccatagatacatcaaggcagcaaaggaggaaactgcctgctccaccggtgaaagctgaaaaaattgagacatcttcagtgactaccattaaaacatttaaccacaacttcattctccaaggctcctcaacaaacaggactaaggaaaggaaaggcaccaccaaggatttgatggagcctgctctggagtcagctgtgatgtactgacaccatggaattccactgccagtgacccactgcctccggccgtacacgacagtgccttgacccaacagccatcgagtactgtatgtatttccacctgaggagaaggcctggggaggccacagtgcaccattgcacagggctgtcctgatacctcatccagaaagccgtctcagacttcagcactgcggtcttgcccactctctgccttaggctcccaggggaatccaagacagaaaatgaagacactggcttccaacagcagcgctccatgtttaagatacatattttccctgtttgctttgctactgtatgttgactttaagatctttttttaaatacatttgattcagctagtattccatgtcaacaatttgtccaaaggaaaactgctggagggaggtggagggaggaaggtgggaattattatttaatacatcattaatgcttattaatctctcacaagcatctttgtcttgcaaatcctaagggaaaagcaagtccctgcagtgagcactagggacagtctaatttggggattgctcaaccatcaagactgcaggtctcccttcagccacctccttcctgctaaaagcttagcctaccacactaccagtcattcccatcgctttgcaatcacaagccacaggatgagaagttctgactcactcatgccatgcccagggctatctgaaacaatgtctcattaagaatttagggttcttccatgggcttactgacagttgcccagatctgaaggggaaagggtcttgagaaagaccatcactggctcaactttagggcactgtccagagtcaacatgatgtggtttagcagtgatcacatctaaacaaagtttaggtaaatgaattatcgcagagaaaaaccacatgagaaaatttttgtactccaaatttacttcccaataaatattcagcaaagtagtaaaatgaccttaaagataaaaatgattagggaatagccttagaaaatttataggtataaaaaattcaaggacaaactgtgcatttaatggacacaagaattgactctaactccatgtctgtggtttctttgaacccatatcaaatgtatgactatttagagtgtttataagagataatggaactgaactttcactcaattaattgggcattaacaaccttcttttatgtttgttcctgatatagtctgaatcttaggaagaaggtaaaagaaaggaggcaagagaatagttatgatgaatatgtgttaagtgcctgctctgaaggaggcaatgttcttctcatttgaatccttatggcaaccttattcaataggttttcccatatttcagatttaataactgaaggccagagagattaatttgccaaagccacacctttatgctaattatgattggaatgcatcacaaaagcctaactctgttgttttcaacctctacgttattttgctgctatgtgcatttccagatctgattttctgctaacttgtgtgctatgatccactcctgatgggggtctacattaatcttccagtactccttgctgatgctgtgttatgtgtcatctaacagaaatgactcctttgaaataagtaaatctttggctttttgttctgttggtgtgattcaaagcaaaacaaacaaacaaaaacaaattttaagaacacaacaaaaaagatttgacttccgaatagaatgttttctttaagaggcatgaaaagcaactattgttgtgttacagtgttaaaaatattcagttttctttgacaaaaatgtgtactgtgtaagccttgcaaacaaaaaacaacaaaaaagaagcagcagcagcagcctgctgtgtggcatctgaacttttataaaggtttccttgtgccaaataagtgcaaagatttaatttactattaaaaaccataagcatatgttatagttccagaagaattattttgtcatcaagtgattttgatctttagtgtcaatatttatatttagattaatttttataaatgaaaatattttaatggtttaagaaaatgaggacaacaggataatatctttgatgacttctgaaagttatgcttcccttcatgttatatgcacattgccaagaattactgtcaagagaaatgataagtaaaagtcatttatgaaaataaaaaaaaaaaaaaaaaaaaaaaaaaa

SEQ ID NO: 56 - RNA, 1473 nt, Homo sapiens
  source 1..1473: mol_type = mRNA
cttcaacactgtggaagctttgttctttcgctcttttgcaataaatcttgctactgctcactctttgggtccacactgcctttatgagctgtaacactcaccgcgaaaggtctgcagcttcactcctgaagccagcgagcccacgagcccacagggaggaatgaacaactccagatgcaccgccttaagagctgtaacactcacggcgaaggtctgcagcttcactcctgagccagcgagaccaggaacccaccagaaggaagaaactccgaacacatccgaacatcagaaggaacaaactccagacggcgccaccttaagagctgtaacactcaccgccagggtccggggcttcgttcttgaagtcagtgagaccaagaacccaccaattccggacgcagtaccatgatcaacaggatgttactagcaacttccttggagcgatgtggttgatatcaataacttttctctccattggttatggtgacatggtacctaacacatactgtggaaaaggagtctgcttacttactggaattatgggtgctggttgcacagccctggtggtagctgtagtggcaaggaagctagaacttaccaaagcagaaaaacacgtgcacaatttcatgatggatactcagctgactaaaagagtaaaaaatgcagctgccaatgtactcagggaaacatggctaatttacaaaaatacaaagctagtgaaaaagatagatcatgcaaaagtaagaaaacatcaacgaaaattcctgcaagctattcatcaattaagaagtgtaaaaatggagcagaggaaactgaatgaccaagcaaacactttggtggacttggcaaagacccagaacatcatgtatgatatgatttctgacttaaacgaaaggagtgaagacttcgagaagaggattgttaccctggaaacaaaactagagactttgattggtagcatccacgccctccctgggctcataagccagaccatcaggcagcagcagagagatttcattgaggctcagatggagagctacgacaagcacgtcacttacaatgctgagcggtcccggtcctcgtccaggaggcggcggtcctcttccacagcaccaccaacttcatcagagagtagctagaagagaataagttaaccacaaaataagactttttgccatcatatggtcaatattttagcttttattgtaaagcccctatggttctaatcagcgttatccgggttctgatgtcagaatcctgggaacctgaacactaagttttaggccaaaatgagtgaaaactctttttttttctttcagatgcacagggaatgcacctattattgctatatagattgttcctcctgtaatttcactaactttttattcatgcacttcaaacaaactttactactacattatatgatatataataaaaaaagttaatttctgcacataaaaaaaaaaaaaaaaa

SEQ ID NO: 57 - RNA, 2112 nt, Homo sapiens
  source 1..2112: mol_type = mRNA
cggcggcagcagcccatgcctccggtgcaacagctgcgcctcctccggtgccccggcggcgggggcgggagataacctgtccctgctgctccgcacctcctcgcccggcggcgccttccggacccgcacctcctcgccgctgtcgggctcgtcctgctgctgctgctgctgctcgtcgcgccggggcagccagctcaatgtgagcgagctgacgccgtccagccatgccagtgcgctccggcagcagtacgcgcagcagtccgcgcagcagtcggcgtccgcctcccagtaccaccagtgccacagcctgcagcccgccgccagccccacgggcagcctcggcagtctgggctccgggcccccgctctcgcaccaccaccaccacccgcacccggcgcaccaccagcaccaccagccccaggcgcgccgcgagagcaaccccttcaccgaaatagccatgagcagctgcaggtacaacgggggcgtcatgcggccgctcagcaacttgagcgcgtcccgccggaacctgcacgagatggactcagaggcgcagcccctgcagccccccgcgtctgtcggaggaggtggcggcgcgtcctccccgtctgcagccgctgccgccgccgccgctgtttcgtcctcagcccccgagatcgtggtgtctaagcccgagcacaacaactccaacaacctggcgctctatggaaccggcggcggaggcagcactggaggaggcggcggcggtggcgggagcgggcacggcagcagcagtggcaccaagtccagcaaaaagaaaaaccagaacatcggctacaagctgggccaccggcgcgccctgttcgaaaagcgcaagcggctcagcgactacgcgctcatcttcggcatgttcggcatcgtggtcatggtcatcgagaccgagctgtcgtggggcgcctacgacaaggcgtcgctgtattccttagctctgaaatgccttatcagtctctccacgatcatcctgctcggtctgatcatcgtgtaccacgccagggaaatacaggtaccatgatcaacaggatgttactagcaacttccttggagcgatgtggttgatatcaataacttttctctccattggttatggtgacatggtacctaacacatactgtggaaaaggagtctgcttacttactggaattatgggtgctggttgcacagccctggtggtagctgtagtggcaaggaagctagaacttaccaaagcagaaaaacacgtgcacaatttcatgatggatactcagctgactaaaagagtaaaaaatgcagctgccaatgtactcagggaaacatggctaatttacaaaaatacaaagctagtgaaaaagatagatcatgcaaaagtaagaaaacatcaacgaaaattcctgcaagctattcatcaattaagaagtgtaaaaatggagcagaggaaactgaatgaccaagcaaacactttggtggacttggcaaagacccagaacatcatgtatgatatgatttctgacttaaacgaaaggagtgaagacttcgagaagaggattgttaccctggaaacaaaactagagactttgattggtagcatccacgccctccctgggctcataagccagaccatcaggcagcagcagagagatttcattgaggctcagatggagagctacgacaagcacgtcacttacaatgctgagcggtcccggtcctcgtccaggaggcggcggtcctcttccacagcaccaccaacttcatcagagagtagctagaagagaataagttaaccacaaaataagactttttgccatcatatggtcaatattttagcttttattgtaaagcccctatggttctaatcagcgttatccgggttctgatgtcagaatcctgggaacctgaacactaagttttaggccaaaatgagtgaaaactctttttttttctttcagatgcacagggaatgcacctattattgctatatagattgttcctcctgtaatttcactaactttttattcatgcacttcaaacaaactttactactacattatatgatatataataaaaaaagttaatttctgcacataaaaaaaaaaaaaaaaa

SEQ ID NO: 58 - DNA, 36151 bp, Homo sapiens
  source 1..36151: mol_type = genomic DNA
caagctgacttctcaagctctggggatgcctcagtgatctctgatgtggaattagtggaggctggccgttgctcacctctacacagcctgctggtcgttagcgagcaggagttctgatgttcctctcatcttggatggtaagaaatttatgaaaatgctaagtcctgaggattaatagaggaaatgaggactaaatatacaaaatagtccagaaggtggcaggtggaacacagcaataatatatgatgttagtcatcagtcattagtgccacagggatcctaggcgatggctgggaaatggcttttgcaggctgagatggtggtagcgcttcaccctctaaggcaagtgcctatgattcaatgggtgaactcactgccaccccagcagggtggggttggggttggcttggtgagaagggagatgtcatcttgcccaaggaggcttatcccttgtgaaagtctggaacaacacacaaaacaccaggaggaactgtttcggagtgctgtcttgaatgcaagcaggcagcctccatttagatctcactgattcttctaggatattacaaagcactttcttaccccaaatccttcagtgccaccctgccactaaatacaaagaagaagctctgcttcccaccgtgcagaggagccacagggattgcatggtccccatgactgcctctttggcccagggaaagggcccaatcagacagcaccccagatcttaagagaagaacttccagtcaaaacaacagggcacccccgtgtgctggaaggtccaactctctccagtcccaagagtgttaagcatctggctaaggctttgctaaatttcccataagagctgcattgttttcttctgtctgttagacctaggaagttttctgggtgtggacgtagggcttttgcaagtttcaaagcttccaggggattagaaaccatcattcccaagcatttacaagaaaatcttttgtccccactgcttgatatcgaaaaacaagggtcactcttcttttcccctaaggagggaaatgggaacaccccgctgaccatctccttctgcctcaggcactttcatggagacagtcgagggctttggggcctgtcctacctggtggtctactcagtagagccatcgggctgaaaaggagacatctgttaaggtaactggtgatcccatgtaaaagctttgactaaaccacaggagctggaaccactttctcagtaaagcatgccaggaagcacaaataaagtcatgtgctgcataaggacgttttggtcaatgagggactgcataaatgatggaggtcccataaaattataatatcatatttttactgtcccttttctatgtttagataaacaaatattaccattgcattacaactgctcacagtatttcagcaggaaagggcccttcgggaggggctccagcttgcctctccaggtctaggtcttatcgttcttccccttgaagttgaccctacaaccttattaaatcttctgcagttaccaaaagtgataatgtgcttggcctttgtttaagctgtgctctctgccttgagctgtcctccttccctctgctcaagtgccccatttttcatgtcccttgggaacaagctcaggtgtcatcactgaattcaggtagatgcccctcttatctgtccccagagcatgctgcacttatccccatcacagcacttctcatgctctgttataatttccttcctattccattagcatatgaggaaccaagggccatgtgtcattcatcctagtacatgttacttagtggagttgcataaatacttatggaatgaataaattgactaagaggtgagctgggctcagcttgtggatgatcctgaaagatagattggcagactcttaccttttttgggagtgtgcaaccggaaactgtttattgtgtttttgaggtgggagataatttattgggactgtcctttggcatgatagctctagaatgagtgctaaggttgaatgggatggaagagagagccagttagtaagccattgcagggtgcaggtgagacagaataaactgaagttaaataggagtaatgcgggaacattcaaaatagggtcagtaagaagtgatcacaaataacgcaagggagacggaggaatcaagggtgatgtggaggtttccagcgtctactgaagtgagtgaatgaggatgcctctttcccactagaggaagataggtatgggggtggccacagctgcccggttgcatggataggaactccttgacttaccttttattagaagatgggtaatgatgactccatcactgtaactgcaatctattctgggtgaagagtgcccatacggtaaatgttttaaaatattctgaatattgcccctggtcccaagggatgaaggcattgtggaggagttttcccttaagcatagttgaaataggagaaaaaaaaaagaaaaggaaaaatcagaagttatgtcttagaagggtgttgatgtggattggtttaccagggttgctgtaacgaagcagcatggcctgggtggcttgaacaacagaggtttattttctcacagttctggaggctagaagtctgagatcaggctgtcagtgggtttgcatcctcctcaggcctctctccgggcatgtaggtggctgtcatctccctgcgtcctcacagggtctttcctgtgtgggtgtctgtgtcctcatctcttcttctaaggacacaagtttgtttttggattgggatccaccctaattacttcacttaactttaatcacctctttaaagacctacctccaaatagggtcacggtgtgagatactgggaagttaggacttcaatatatgcattttggtggcaggaggcgcacaattcagcccgcaacatgaggcatgagcaaaaatataaatttaaacaatgttggcaaaaacagattcaaaagcaaaatgcaacgccaaagcatgaggaaagggaagaagttttggatagtaggcatggatgaggtggtcccgagggagagaagaggctggggcaccacgcggaggggtttggtagaccctgagtggtttctctcattgtagagggtcaggaacaatttgaacacagaacaactgaagagccaggcttgatggctctgttaaggaaaagacacttttaggacgtgcagttggaccttttcaagaagctgttggaggtcagggtctgagttgagaaaggagcttggtcctgtaagtcatcgacactcagttgcttctggatcccatcctcaccccctgcaccgcggggtccttctcagggaacgggccacttcccgatgctccctgggctgccatcagctgccatgaacactctgcttagtggaggctccgtggcctgtgaattagcatcaacctgaaataacaagcaggggcttcctatgagtcagccatagtctgttcagagatgctgcctaaagggtctaaatccaaaggcattaattaatttgttcccagggtccacagcatggggagctcaagcccttccatttccgctcattgataccggctgggtaatattatgtgggttccatttttaatgtagaatgccttatttgacaaacatccagacagcacccccaactttgttttcttgagcagaagagagaaaacaagagatgaaggcattgatgcatcagccctgcagaaaaatggcaaaggttacaatatgttatatttagatccgcaatttgccgaaggaagataattgggaaattaagttgcctggttgggaaatcagccgtccttttgtttttgtccggttgtgtagtctcataaatgttcagggcttcaatacctttaggcacttataattaattttttaaaaattggaagattcagtatataagctgcctgatttaatgaaataagtcaaaaggagaaaaaagaagggagtttcttgcagtaattggttgttacatttttccacattgccattctaattcattcctttgaaaagagttcgtttttcagattctataagtacccatggcaataagtgctatttataataaagagacataatgagtctcagctgcacccagggaggttgcagcaggtcaggagcctctctgtctgctccattgttgctgggagagagggtcttgttccaagggttgggtgggaaacaggaattccccacactgtccctgaaattgtggagtgatgaactagggagggaagtcttaactggaatttttcaatattagattttccagagacttcattcagcccactcttcagtagggaaataaatcctttatgtagaaaaaaaatcaaaccatttccttttgaataaggtaatctctgaatctgaggaaccccgagataagatgatgtattagcctgctcaggctgtcataacaatgtaccgcagactgggtggcttcaacgtcggagatttatttccttgcagttccggaggccgggagttctagaatacatcaaggcatccacagggttggtgtcttctgagtcctctctcccgggcatgcagggggctgtcatctccctgtgtcctcacatggtcttcaccctgtgtatgtgtgccttactctttgcttaaaaggacatcagtcatattgggttagggtagagcctcattttaccttcactaccattttaaataccctatctccaaacacagtcacattctgaggtactggctctgcatataaatttggggggacacatttcagctcataatagatgtgcatcttatttttgaaaataaaattacagaggtctttcctcttattcccttttcctctttcaccattcctgcctttcctgtattagctgagacagtaattcatattgtcagagcacacactgtctctctctctcacacacaaacacacatacctccacacactttatctttcctgcctccaaattattttcttgttattggaagcaaaaatgcatttttccttcataggcaccttcttacccagtcctcaatgggtgctgtgatcaaaggaggccaccacagctctattagctcatgcccaaataaagccaaatctcacaccttccacaagtgacctgtgttgctcccttcctgaggttgtttcacagagaagagacccaaggttaggctggctgttggcctccatcagtgatagtgatggctgggtcacaggttgctgaaggagtcaagggactcattcaatactggatgcttctgttcctctttcctctggggctggactaattaaggaaaaatgtttcctggctgttcaggtttcctccatgctaattcaggcctattcttgcagatgcatacagttatgacttcattatgcttgttggtatggaaatagaggtaagatcaggtgcccagtggtctgaaaaatgttgacatgcatttttgagcctgtccacagttgtcgttaaagccttgtcagacttccctgggaggacatttagagtgagaaaaaagagaacctaggaaaaattcctgggaaacatcagcatttccagttcagctgcatgcttgcagatgcgaaaaagtcagggaagtggaagccaggaaggtgagaggaaaaccgggatgggaggagctgtggagatcaagggtggggcaagagtttcatcggggaggggcagcagtctgcagtggccagggttgtggatacatgaagcatatgattcctgactaagtgaggatttctggagtgcgtcatctaattttctctgaactcattttgcaagaatttcctagggattccattggggcgggcgaggttccccttgatgctttcatgtagtagttggctcagagggaatggtggaggtaggaggcatatagctcctcttcaagtgccacttcaaatccactaggtgttatctctcattttggctactgagctacatgttgtgcccccgttccagcaccttccagcaggtgtaactgacaatactcaccccaggctcacacacatagcctttgctttgtgctgctgtcttctccattggtatggcatggagcatggccattagcacctgctcagtgctcatgagtgcaccagagttatttccctgtgggcagcttgtgaccagagtaggtcagaagctaatggagaaatgcttcctcctttcctcctctgggaaaaaggtctgcagaattgagcaactggttgcctgtggtgatggccaaatttctgaacacatcattagaatgttagggtgactctctcctcctctgctttgctttctctcacccttggtccatggaccaccctccctactgaagtacttgcaactcaactttgggggaaactgtggataagagaattagtctgggaagagttggattacttaaaagtcaaagaggaatacggaccccattgctggtgataagaggagtggtgataatccttggtatgcaacagcttcctgttactatggctcttgcctgtggtagagtgatgtgtggcacaggtggaaggtaatgcattaacttaggtcagcagctggcaagccatcgtccgtcgcctatttttgtatggcctgtgagtataaaatggcttttgcatttttagaggattggaaccacacacacacacacacacatacacacacacacacacacacacacagaatatgcaacagagactgtgtgtagcctacaaagcctaaaatattttctgtcttccatttactgacgaagtttgctgaactctggtgtaagtgatagggtaccatgtgaacaatgagaacatgaagaatggccatggcagctgacagctaacggctagatagtactagtgaacatgagtaacactcacagaatataagcaataattagacaaggccactgtgtcaccaccatggaacaagacagacaagaccacttcatgactgtgagtgaaacaagatagagacaaggacaccctgcaactccccctccaaataacttaacatgccactcttctaacaagaataagagactatcacttcgtgctgattgcaactctatctgtgattcaatcctcctgcctcctaattaaaaattaccaaggaaccaaatcactgaattgttcacataccctaacagcattcagtctagaactgtctttgtttccttcaactctccctccaatcacctggcacaaggtcaaattctacaaaaatcctatcttaattcctagtttccaagacatcccacagccctacagaaataaagaccacccaactgagaacaaggatggtgatttattcggagcttgctgtagtgaaagagtcggccaccatcactggtgtttggcagagactcaaaggcaggcagaggggtgggagagctttacagtgggcaaaaaagacgtcttcaggtgtgccccaattggaggctgttggcctagagaagctggaggtggccaagtaggagcagggcaggctatgtgattggttggggacatatttgaccttctctggtttgtcctgaattggaagtggggacaaaaatcaagaaatatgtcagctattaatcatgtgctggtcattttgggctgattgctacagaagttgtggctcagagtcttattgccacctctggtccagccactggccatgggtgtgttcagttttcagttctctggaatgccttcttttctctgcagtaagctcaatgaacttggaagcagattctcccccacagcctctagggaagagcccagctatgtcaatgctgatttgggcctgtgagaccctaagctgagaacacagttaaggccccccagacttctgatctccagagttagaaataaatgagcactctttcaagctgctgagtttatgctaatttgtttgacaacaggtgattcctgagggtcactggttactggacttggaaaactgtgtgagtgcaagcataattattaggatcacagtgtatgctgggttttgttaactgcattgacagccttaaagaaaatggtaccctcagatcagccaactgtcaactcaaggtgtgctgtgaaagctggaggcctgtagaaagagtttaaagaaccatgacctcctttagccagaggaagactagatatctacaaagacctcacttgaggctgatccttcctcacaagatggtgttcatcctcctgagggtctttgctgtgaccccttaccatctccagaccaatgaccaggatcaggcctcatagccctgcctgccctgaaaacatagggataaaacagatcccagtggttatttagcaaatgttctgcatcctctatgtctgcatcagttactacctcaaaacagctggcattttgccttttttcccccactaatttaaagcgcattctgagagcaggggtcagcacactctttctttaagggtcagattgtaaatattttcatttctgcaaaccatttggtctttgttgttatcctccactttgccactagtagacatagataatacacaagcgaatgggcatggcttttgttccaatataacttggtttacaaaaacagaaacaggccagatttggcccacaggctatagtgttccagcttcagtcttggaaaatcatttaatctctctgggtttcatttctacacctgtaaaattagagtattgttctaagacaatgcctctcacactccagctagcgttagaatcatgtggagagctactgaaagcacagattcttgcaccttactcccagtgattccagttgaataggtctggagtgggatctgcatttttaacatgctcccaggtaaaactagtgccgctggtccagggaccatgctttgagtagcagaagtcttgggcagtacttctcatccctggcttcagaggagactcgtgctgagaacttttaaaaagatactgatccctctgttccacctaagaccaattaaattacaagcagaatccaggcttctgagcattttaaaatttcccccaagggatcatgtacaatcagggttgagatccactggtggaggtgagctccacagccctgccctccaagtatgattcttgatgagtggccttggcatcagcacaacgtaggaacttcccagtaacacacagtctcgggctccaccttgacctactgaatcagaatccacattataacaagatcccttggtgatgcatgaacacattaaagttggggaaatggggttctacaggaaggaatggatgaatattttcacaggttcctcttctcctactacacaccaggcactgggtgggctatacttaagtcacttaaacttgcctgtgtaggttttctactgctgtcctgacagattaacataaacttagtaacttaaagcaacactcatttattttctcacagttctgtggattggcagtctggggaggctcaagtgggttctctgctcaggatctcacaggcccagagcagggtattgctgtgttaggttcttaactcaggcttggggaaggaatccacttctaagctcaattacgtttttggcagaatcaagcttcttgcagttgtaggactgaggttgctgttttcttgtgagctgttggccaggggttgccttctgcttctagatgtctctttgagttccttttaacttttacattatgttcaaagatagcaaccattcatcatatcctcctcatgcttccaacctctctgactcctccttccgctactagctagtgaaaacttgcttttaaagagctcgtgtgattatattaaactcacctccataatcgccaattttactaattcaaggttgatggattagtaaccttaataacatctgtaaaattcctttttccataaatgtgttatctcgtatattcccagtcactggcattagggctgaaggtcttgggggacatttaagaattctgtctacctcaatgtaccatgccacttacacgtcagtcttagagtcagattttgaatctaggtgtgtctgatcctaaggcttatatgaacattattttaccaatttgtttctctgtggtaagaacaaaggtgttctttgttcagagaaatttatagttatcaaagagctttcacatttattttattgtttggtctcataaaagacctttattaggtattattattctttttatgggggattgagaatgtttaattatttgttcaagggcctataagcagaatatagaaaattcaggactgaagaatgggtctcctgattccaaatttcatacttttccttttatattaccacaattttataatttcttatttagatcattcttgttgtctaattagaaaacaaatttaccaggatagaataaaagaaactagcagagtagttgtagataggatgcaaatgtctgaatcctgcagtgggccctgcacagaaaaatagacccacctgttacgctaattgaaggaaaaactctgctgaaattcagagaagtactccattcaggtcttaaaaattattgttcttaaagaagacagtcattcaatttttaaatgtaatttagaaaacaaccaattcacaaaacaaacactttttactgaattacaagtcaatcatgaccttactttggagaaatgacaggaaatattggcaaatcatgtggatgcaactcacatttgtgataagaaagttagcctgctcccatggatgaagcttaaggagaatcataaaaccatcctccttctagtgactatctttcagttcattctctttttgtttcaaaagcaaagaaaccaatgtgacctgaatgaactataattacaaagctaagattctttcagagggcaggatgatatctactggttggggttttcttggcatggccaactaggattctggtatatagggcatgtctctcatgagtcccctaataatgtaatcattttttcttctccatatggtgcaaaagtcatatctagtgagaaagtatccttacattgccagtttagaagttattttatgcttaaaaaatggcccagtcagttgttcaaataaaaaacagctggaaattcaatatcttgggctgtttccttgtcatcatttatagatgtaaggggtttcattacaatctgagaggcagtctatgatttaaacaaattatcaaaaaaattaggccttggtcagctcaattttattaaaagccttcagatcttaaaggatcaatgactacaagagaaaggttaacccccagggctcctggttggtgaacaaacacatcaaagtgaaatcagagttaaaagctattgttcccttggctaacttttgcctctgtcatattcagttgatttgaagagatgaggaatgataaacaggaccaggttacaaacatagaactgttacattttgttcatttagccaggagctttattcttttgtttttcatgagatgggtcattttaatagtcaacggtgttgacgacagtcaccattcaatctgtcatttctaatacagtctttcctaggagaatatttctcacgtgtttgacagaaaacattatctgtccatcatttccatggattcctgggtctcacgggcagaaggacaaggcagatatgaggcggatctggtaaagtaagtcattgtgtactctttgtatttgtttgaagatatttggtctttttcagtcatatatgtgatggagaaccagttgttagaacaatgtgtatattgatggagaaacaggtgggccagagtggtcggtagtctgtgtgacaaacagtgatgccactttcactatctgaaaatgaatgaatatattcatagtagtgggcatgcaaaggtaataaagacctgacccccaacatcaataatataataatgatgaattcatattctaaatctgatcatttaaggaaggccatccatatctccatcacaatcacttaaacaaatgcaaaaatggatattctttctagaatatttctgatgaaattgcttctgaaggctgcaaattagtgctcaagaccctgttgaaaagcagtttcctgtattcacttatgaagtcaaatgcagaaattatatttatttggattttatttatagcagagagaataacttaaaaactgacatagcattcttttcaaatctgaactcaatgtctgatttttggaagattcatatcagctctactcttataaatcagaaaacaaaaacaaaacaaacaaacaaaaaacaacaacaaaaaaagaaaaacaggcccagacaccagcagtgacccagtcaaccactggccagtcaatatgagagccagctggttcttctagtgaaaccttcacctttctaatcagagggtttcaggttttgctctctattgagtcaattgcttgagggtagaaaaagaaaatcttcgaattgcattacataccactcctttccctttctatagatttccccctctctcctttccaggaatattgattttcttccctgggtttctgaaaaagcttttctccccaggcatctttatttggtttgctgaccaaagcattattcacacttctcctgcggccacgccaatgccacacccgctgcaaggtgctcccggtctcctacctctgaacaacagtggtctgaatctctgacttgagccgtgcagccctttgaaagaaaagtcatttaggagctcagccctctgaggatgtaaagcccgtcgcaggtgcttgttttgtctgtctccgtgtgtggaagatgccttctgtgaggtcctgcgcctctccgtaagcagctgagtagttccttgggctgacaaaatactttgttgtggctccattacacacccagggcaacctctttagctggattttgcagaccgggaaatgggctctgaggggaagtcacgcctcatctcaaggtttactgaggagccacgaggaggccccaccgctgccacagctaactcatggcctccggccaggaagtctggagattttaagaccataatgtcagccagggtgacctctggaacctgttcaccccttcacccgttgtgaacaagagagctgaatagccactatcacagataataacaacagctgacattattaataggatgccttacaggctgtaaaatactttcacaaacaggatctcatttgatcctcataatctcaacatgaggtgcgtaaagcaagcaattattactaatctccttttacagaagagaaaaggaagtcctgagagatgaatccacttctgggaggtctgacaacgttcagctttacctaagccatggtcaggaaacttttctgcgttccaggaaatggccggccacaacgactggcccttccctataaaaacttacaaaaccctcacaggtgacctccttgtttacctatgacagaaccagacacagtgccagatcctccaaactcccattctttgccttatatgtaatcagttgaactgtttgtatctactgactaatctagacaaggtacttgctaacttgacctgtttaaaagttcgaaatgccaaactttctttcttttttttttttttcccatcccaccccacccagatggagtttcactcttgtcacccaggctggagtgagtgcagtggtgccatctgggctcagtgcaacctctgcctcccgggttcaagcaattctcctgcctcagcctctagaatagctgggattacaggcatgtgccaccacacccggctaattttgtttgtttagtagagacagggtttcatcatgttggccaggcttgttacaaactcctgacctgaggtgatctgctgcctcagcctcccacagtgctgggattacaagtgtgagccacagtgcctggcccgaaatgctaaactttcatcaagatattgcaatagtcttttctaagaaagctcttccttacttaacttgtgcctggccccaaatgctaaactttcatcaaatattgcaatagtcttttctaagaaagctcttccttacttaacttgggatttgttttatctgacacagggcacagggtaaagatcagaggtggcttcctgtcactcatccagccagccacccagctatggttacccacagggcctgatagccatccctgccagcggtcagggttgcattgtggcccccactccaggccttgcttattgcaggcttggttcccatggctgctctctgtgttcttgcaaccttgcatcctgccagaatctctggaagaaagctctttggcgagaaatgaagaacaatcccaaaggtgctccaggaaagttcattattttgtgtactctggtgtcttaccttaatcagagaacagttgtctgtgatcctcaaagagggaattaaagggaggaatggaaccagaattcagtgacaactggaatgagtgactgaggtgtagcgtcaatcatcaaggtttattaaactacttagggtgcgtctggaaaaaacatgagccacaaatacatctggggttgtttttccaaagaggtttttgggagatttagtatttatacatttccttagagggaaaaggcatgtggaaagaggggcaggtaggcagttggcaaatactacattttacataagataaggcaaatgaagagagaaagagagtaaaggaatagtccattttacttatgtctttgttctgcacctgggaggaagttcttcatgacatcagtgtggaatcaacaaactttagttttaggagctaggcttagtttgtagacctacagttataactggtggttctgttgatgggatgccagtgaagaatgtacttaggaatgatctgtgaggactgtcctttcctgatgcctgagtcctttttcttggggtgagcggggggtacaaaataggagcatatatttatggagtacatgagatgttttgatacgggcatgcaatgtgtaataatcatgtcatggacaatgaggtatccatcccctcaagcatttatcctttcttttatgaacaatctgattacacttttttacttattttaaaatgaacaattaagttattattgactataatcaccttattgccaatgtttgtcattctgtgcctggcttatttcacttgatagaatgttcaccagttccatccatgttgctgcaaatgtcaggatctcattctcttttaagactgaatagtgctccattgtgtatatgtagcacattttctttatccattcatgtgttgatggacacaggttgcttccaaatcttagctattgtgaacagggctgcaacaaacatgagagtgcagaaatctcttgcatatactgatttcctttgttttgggtgtatacccaacagtgggattgctggaccatatggtagctctatttttagttttttgaggaactttcaaactgttctccattgtggttgtactaatttatattccaactaacagtgtaggagtgttccctttcctccacatcctcgccagcatttgttactgcctgtcttttggatataagccattttaactggggtgagatgatatctcatggtagttttgttttgcatttctctgatgatcaatgatgttgagcacttttttgtatgcctgtttgccatttgcatgtcttcttctgagaaacctctattcaaatcttatgtccagttttttgatcagattattagactttttcctatagagttgtttgagttctttatatatggcgcttataaatcccttgtcagatcggtagtttgcaaatattttctcccattatgtaggttgcctcctcactttgttgattgtttcctttgctgtgcagaaggtttttaccttaatgtaatcccatttgtccacttctgctttggttgccttgcttgctgggtattactcaagaaatttttacccaaaccaatgtcctggagagttttttctatgttttcttgtagtaatttcatagtttgaggtcttagatttaagtctttaattcattttgatttaatttttgtatatgaccagagataggggtctagttttttcctgcatatgaatatccagtttccccagaaccatttattgaagaaactgtcttttccccagtgtatgttcttggcacctttattgaaaattcacatattgtaggtgtgtggatttatttctgtgttttctattctgtttcattggtctatgtgtctgttttaatgccagtaccatgctattttggttactatggttctgcagtataatttgaagtcaggtaatgtgattcttccagttttgttctatttgcttaggacagctttggctattctgggtgttttgtggttccacataaattttaggattgttttttctatttctctaaagaagttcagtggtattttgacaggtattgcattgaatctgtagattgatttgggtagtatggacagtttaataatattgattcttccaatttatgaacatggaatatctttccgtttttttggtgttgtcttcagtttctttcatcagtgttttgttgtttcattatagagatctgttgcttctttaggttaattcctaggtttttaattttacttttggcttttgtaaatgggatttacaaaaatttctttttcaggttgttcactgttggtatacaggaatgctaatgatttctgtacgttgattttatatcctgcaactttactaaatttgtttatcagttttaatagttttttggtggagtctttaggtttttccaaatataagatcatatcatttgcaaacaaggagaatttcacttctttctttccaatttggatgccatttatttctttcttctgattgctttagctaggaattccagtagtatgttggataatactagtgaaagtgggcatccttgtcatggtcatgttccatatcttagaaaaaagctttcagtttttccccattcaatatgatacaagctgtgggtctgtcatatatggcttttattatgttgaggtgtgtttcttctacacccagtttttttagggtttcttatcatgaaatgatgttgaattttattgaatgcttttttggcatcaattcaaaagatccttatatcctttattctgttttatcctttatcctttattctgttgacatgatgtatcatgttgattaatatacatatgttgaaaaattcttgcctcccagggataaatcccacttggttatgatgaatgatctttctaatgtgttgttgaattcagtttgctagtattttgttgaggacttaaaggccagagattttggcctgtggttttctttttttgagtgtctttgtctggttttgcattatagaatgaatttggaagtattcacttctcctctatttttcaaaatggtttgagtaggattagtattagttcttccttaaatgtttggtagaattcagcagtaaagccattgggtcctgggcttttctttactgagagaatttttatttgatctcattacttgttattggtttgtttaggttttgaatttcttactggcacaagcttggtaggttttatgtgtctagaaatttgtccatttcttggagaaattccaatttattggcctatagttgcttatagtagccactaatgatcctttgaatttttgcagtatcagttgtgaagtctcctatttcatttctgattttatgtttttgtacctctattttttaaattagtttgactaaaaggtttgtcaattgtgtttaactataaaaagaacctttttgttttattgatcctttttattattttttttcatttcaatttcacttatttctgctctgatctttattacttttcttttactaattttgagtttggtgtgctcttgtttttctatttctttaagatgcatcattagatcacttatttgaaggatttccccttttttgatgtaagcatttgtagatataaacttcatcttagtactgcttttgctgtatcccataggttttggtatgttgtttccattaacatctgtttcaagacatttttcaatttccctcttaatttctccattgatccactagtcattcaggagcatattgcttaatttccatgtgtttgtgtacttcccaaaattcctcatgttattgatttctagttttattccattgtggtcagagaagatgcttggaattattttaattttttgaatgttttaagatttgtttttcacctaatgtatggtctacgcttgagaatgatccttgcactgaggaaaagaatgtatattctgcagccattggatgaaatgttctataaatatctattagatccatttggcctatagtgaagattaagtcggatgattcttggttgattttctgtctaaaagatctgtccaatgctgaaagtggggtgttgaagtctccagctattctcgtattagagtctatctttctctttagctctgacatttgccttatacatctggatgctccagtgttggggacatatatatctaaaattgttattcatatattatcttgctgaattggccctttaatcattatatagtgaccttgtttgtcatttcttatagattttgtctcaaaaatctattttgtctgaaaatatagtgactcctgcttatatcagacaaatttgatatttttccatccctttattttcagtctgtgtgtgtctttataggtggtgtgtttcctataggcaagatatcagcaggtcttgcttttttcattcatttagccaggctatgccttttaatttgggagtttagtccatttatattcaatgttattattgataagtaaagacttcctcctgccattttgttattgttttctggttgttttgtggtcttctcttccttctctctttttcttcctgtattcaattaactgaaggtgattttctctggtgatatgatttagtttcttgcttttccttttttgtgtatccattgtatgttttttggtttgaagttaccatgaggcctgcaaatactagcttataacccgtgattgtaacccaatagcaacttaaccactgtttgcataaataaacaagcaaaaagataactaataaacactctacacatttttttttttttttgagacagtgtctttgctctgttgcccaggctggagtgcagtggtgcaatctcagctcacggctctttctgcctccatattcaagtgattctcatacttcagtctcccaagtagtggagattatagtcatgtgccagcatgcccagctgatttttgtgtttttagtagagatggggtttcaccatattggccagtctggtctcgaactcctgacctcaagtgatccgcctgcctcagcctcccaaagtgctgggtttataggcataagccacaatacctggccaacattctacatcttaacttcatccccccactttttaacgttttgttttatctatttacattttcttatactgtctatgtcttgaaaagttgttctagttactatttttgactggttcatcatttagtctttctacttagaataaaagtagtttacacaccacagttacagtgttgtaatagtctgtgtttgtatacttaccattaacagtgagttttgtatcttcaggtgattacttattgttcattaacacccttttctttctgattgaagtactccctttagcatttcttgtaggacaggtctagtgttgatgaaatcctcagcttttgtttgtctgggaaagtctttatttctctttcatgtttgaggggtattttttgccagatatactgttctggggtgtacattttcttctttcagcactttaaatatgtcatgctactctctcctggcctgtaagatttccattgaaaagtctgctgccagaagcattggtgcttcgttgtatgttaattgttccttttctcttgttgcttttaggatcctttatccttgatctttatcctgaatttgattattaaatgccttgaagtggtcttcttcgggttaaatctgcttggtgttctataactttcttgtacttggatattgatatctctctctaggtttgggaaattctctgatatccctttaaataaactttctgcccctatgtctttctctgcctcttctttaaggtcaataactctcagatgttcccttttgaggctattttataaatcctgtaggtgtgcttaattgtattttattcttttttcttttgtctcctctggctgtgtattttcaaacagcctatcttcaggctcactaattctttcttctgcttgatcagttctgctattaaaagactctgatgcattcttcagtatgccagttgcatttttcagctccagaatttctgcttgattctttttaattacttcagtctctgttaaatttccctgatagaattctgaattccttctctgtgttatcttgaatttctttgagtttcctcaatatagctattttgaattctctgtctaaaaggtcacatatctctgtttcattaggattggtccctggtgccttatttagttcatttgataaggtcatgatttcctggatggtgctgatgctagatgttcttcggtgtctgggcattgaagagttaggtatttattgttgtcttcactgtctgggcttgtttgtacccatccttcttgggaaggctccagatacttaaagaactagcgtgttgtgatataagctgtatctgctttagggggcacccaagcccagtaacactgtggttcttgcagactagcagagatataccatctggtatctcttggtattgatggtcgtggacaagatcgaggagaattctctagattaccaggcagagactcttgttctcttcccttacttcctcccaaacaaacagggtctctctatctctgttctgagccacctaaagctggaggtgaagtgacacaagcacccctgtggctaccatcattatgacggcacttggttgaactgaagccagcacagcactgggtctcacccaaggtctgctgtaacctctctctggccactgcctatgttcactcaaggcccttgggctttagagtaagcagatggcaaagccaaccaggcctttgtcttttccttcagggcagcaagtttgcccaggcccctggagggtctagaggtgccatctgggagtcagggactacagtcaaaaaccttagaagtctacttggtgttctactgtattgcagctgagctggcactcaaaccacaagacacagtccttcctgctcttccttcccctttccaaaggcaaaggagcctcaccctgaagtccctgccaccacaggtcacagggagtactgccagactactgccgatgatcccttaagacccaagagctcttcagtcagcttgtggtgaatgctgcctggcctgggactcacccttcagggtagtgggctcccttctggacacaggcaggtccagaaatgccatccaagagtcaagttctggaataagggaccccaggagcccacttggtgctctacccttctgtggccaaaccagcacctaaggtgaaggacaaagtcccctttacttttccttctgcttttctcaagcagaaggactctcttactagagctaccatagctgggaatgtgccgagtctcacctgaagccagcaagtctcagaggctcaccaaggccctcaacatagttcctgggtattgctgctgtttattcagggcccacgggctcttcagttagaaggtgttgagtgctggcaggactgagttcttcccttcaagacagcaggttcccttctggcccgggatgtgtctagaaatgtcatccaggcgccaggtcctggatcaaagccctcacaactgtgactggtatcctatcctgctgtggcttagccggtatccaagatacaagacaaagtcctccccacccttccctctcctctcctcaagcagaaggaaggggtctcttttggagccacaagctgtgcagcctggggttaggggaggggcgatgccagtactcccttgtcttccccagcttgtgtctcagtatgtcgcccatccccagtccactgtctctgggcctggttcagcagtagctcttgcctaagagttgcagttcctgtggcctagactgcttttcaagtttacttggagatacagagcgctgtagccctcagtggtgaggtttgcagaaactcaagtttgcacggctgggatctgtgatcctttctggctagggctggtttaaatgttccctctgtgggtgagagtgagctgagttaggtcctttcggttttcctttctgctctaacaggacagcactgagttcagtgcttcacaattgctgtgctctccctcctctagtgcctagagatgctctccacaccacatcacaacggtttttcctgcctctgaagttaaaaccaggtactatgagggctcacctgatttttggttctgatgaaggttgtgtgtgtgtgtgtgtgtgtgtgtgtgtgtgtgtgtgtgtgtgtaaattggtgtccttgaagggaggatgatgggtggagtcttctattttgccatcttgctccacctccacctcacctgaggccttttacctctctctgaggacccgcctaatgcgtaatgctggtaacaggtattcatttgggagaaggtgttgcagtgatcttcccttttgcataagaattttggaggggtcctgagatactttaattaattgaagaataatcccaaatgttctccaggaaagtttatgttgttttgtttatgctggtggtttaccttaacccagaaaagttgctgtgatccaaaaaggagcaagttaaagggaggaatgaaacaggaattcaggatgaaaaaatgagaaatctttccaatggttgttgttttgtttatgatgaatgccacggatatcagacattcttatgcagaagtgagtttaaaagatccaaagcatttccaccgttttctgtttaaagagcagcaacaagaatcacacatgttccaacatgcagtttagagaggctagaggcacagatatggacagacgtccctgctcctaacagctctctaaggtctggtggaggattcagacacatgacctggcctggaggattgagcttctggattgtctgtaagctaggcaatccactatttagtctttagtactgtaatagaggtctcagtaagtgtgtaccttgtttgcctactttatatatttttattttgtgcgataccaaatattgtggaaggatttcaggtaggaattttgagtgttgtttttgcatcaatttataaagatgacggtctctgagctttgcaggcagtatgtctataccgtgttgattcatgtgcctggctacaggtcaccacacactatacactcttgagtttgctctggagaaatctcaatgagccacctgcaacataacctgctttgtggctgggttccttaaaatccaatatattctgaaggatgtgatcaataagcagatggctcaagcccatacacatgccaagaaatgaggcatctaattaacagttagacaagggtgtgagggaattgcagaggggtgtggaggccctgcctgggctgggagacctcgggatgggagaatcagggctggttgtgtgacgtgtcgacacatagtggctgttggaagtgtgaacacagtcgggaccttcaggctgtgctcattagggctttgcaaaattcccaattatctcttgacctggccctaagcaaggcccctttccattgccctggaaaaaaaaaatgagtgagtcaaaaaggacacctgatttgacacgctacatgggtttggttctgaagctaggctagtagaacacagaaatgaggcattcttgtgcaaccagtgatgactacaggaaggataagtaccaggagcatccggtgtcacagaggcaagggcgggctttttgagatggctgtatggtgccccaaacaaaggccagaaagagacgatttgtgagaagagaccaacgcgccccttgcaaagaattcataagcagtgtttcagttaaactgcaaatctcatggatggtgtcaaaagagtgaggactcctttccgtacacgtctcacagctcaaacaggaacagaaagaggctttctctctgtgtcttggagaacccaccactaagtgctccctggtgtcagagaagggaattgggctcagtgtgacagctttccctgctatatgtgaaatacattatcaatctgttgtcagcctgatgctccctagctgactgttttgttgttgcgcttccagtgttaccctgggttggtccctgcgtgatgtttctaatataaaatatgcagattgtgaaagagaagacaatgcttcgagtctgatctatcctaagatgaatgtgctaatgctcccaagacccataagacagaggaggtcaagaaggtgccttgtggtaggcgatgcatcttcaaattcgttgacacaggctcgtgaattcagattagcacttgaccacaaatacacatgttaaaacaaagtcacattaaacatctctcttcctcaatctcctttcccagcttccttgcagagaagaaatgcaagaaggtgagtgaataaaatcagaattatttgctttttattctgaggacaaaagacaactggttggattccatgggcacacagtgatctaaggttatagtaaccaacaactatgactaagaaagtgatagtcattgtgatagttaattttacatgtccatttggctaggctatggtgcccaattgttttatcaaatgctagctggatgttgctgtgaaggtgattaacatttaaatccataagctttgagtaaagttgattaccgtccataatgtgagtggccctcatccagttagctgaaggacttacgagaatgattgaggtttctggaagaagaaattctgcctcaagacttttaacatagagactctcagcctgcagattttggactcaagactgcaacatcatcttgtgcctgaaattccagtaggctgccttgccctacagattttggacttgtcatcatcccccataatgacatgagtcaattccttaacataaatctctcctatatatcctactggctctgtttttctggataaccctgattaacacaatcattctgtctattgtagatgatctgtcctccttgaccacactcattggccaacactagccttattacctgcaaacatgagatagcatgatctattgcccagaaaagaggatagaatagaggctatttttttcctcccatctccctgttcccctgtcctctatccttatcttaatctttccaacctactattatgctccaagagtgttacctacttttattattttctgtcttcaaagttaaagagtgcctgaaagtatgcagataattcaaacaaaaaagcaacacttccctgtgtaattattcgctcaactctttgccttggtgtaatttatgtgctcaacacttttccttggaatcttagctaaagaagacttttctggggactgggcgtggtggctgaagcctgtaatcccagcactttgggaggccgaggtgggcggatcacgaggtcaggagatcaagaccatcctggctaacacggtgaaaccccatctctactaaaaatacaaaaaaattagccaggtgtggtggcggacacctgtagtcccagctattcaggaggctgaggcaggagaatggcgtgaacccaggaggcagaacttgcagtgagccaagatcgcatcattgcactccagcctgggcgacagagcaagactctgtctcaaaaaaaaaaaaaaaaagaacacttctggaaactgtttttcctggcagtccttggtcaagttgctaatttaatattttagatggttttgacatggtttagaatgcaaaattatcatcttttaatatgtatttgtttgcagagaacccatgggccttagttcatttctgcacccacagagaggtttaatctcatttaaatgctaaacatcgcagtggtcaggcattaggaacactgctaggaaggctgcaacaaaacaagtgtttccacttagtgttatttgggggttaccatccacatcatacaggaaatggagtgacaaataccaatttgagaaaggaacacaatgaaactacataattcctcttgtaggttagttgatctgtaaccaagccttaaaaattgacattgacattgaaccactgcctgtgaagggagaatgagaaataacctcaaatctgcctctaaataaaccatccatagcttaattcaagagttgggataccagctctctagcattcaaatggaatttaggtatttactaggcatactgtgggggatttttaagttgaatgggtaacatagagcattggggcaaaataatagaatacggttgctggtttcaatgacagcatgggtcttttccaccaagagactacagtacaaaatttaaatcaggtaaactggtccaatgtagaaagcaggcagtcagggaagcatagtttaagggtggcattgaaatagttttgccacttcaatttggtcactagcttttgggatcagagtatagtgtcaatgaagagagtcaaattctgtaaaatatttgaagagatttattctgagccaaatatgagtgaccatggctcatgacagccctcaggagaccctgagaacatgtacccaaggtagttggggcacagcttggttttatacactttagggagacacaagacatcaatcaaatacatttaagatatacattggttcgattcagaaaggtggtacaacctgaagtggcaggggggtggtggcggggcagcttccaagttataggtagatttaaacattttctgattggcaattggttgaaagacttactatcaatagaaagaaatgtctgagttatgataaaatgttgctgagaccaaagttttatcatgcagatgaagcctccaggtagcaggctctagagagaatagattgtaaatatttcttatcagacttaaggtctgtgttgatgttacatgctggtcagctcttcctgaattccaaaagggaggtggtataacgaggtatgtctaacctctctaagagctccctcttagaaacaaacagacaaaacacacaacaaaaaccacacttgtaaacccatcgtcccatctctccacagcttgacttcttttcataatccagatttggcttagggatccaagagtcttgaaaatatttcccatgcatttattctgaaatttgccaagggtaatttgactcctccctttgggttttgtgtttatggtgcctgagatttcaaatttgacattgttaattttgttccccacctccaatcagccttgcccacctcaacaaatgtccacttcattctttcggtgggggtcactctggactttgttctttctctcaaattgtcagtcaacaactctgtcggccctgcctttgaaataaattcatctaggatcctgcccctcttcaccactgccaccaccatgctggctaaagccactgttatttctcgtaggttaggacaacaccttctgtctggtgttgcctctactgtctagtctccacacatagccagaatactcttaaaacatacatcagataccatcatttcttagcttgccattctctaatgatgtcccacaattcttagagtaacatctcaaaatgtgaccatgccctgtgtattagtcgtcctcatcctcctcctcctcctccccttcttcttcttcttcttctttatcatcattaaactttaagttctgggatagatgtgcagaatatgcaggtttattacataggtatacatgtgccatgtggttttctgcacccgtcaacccattgtctaggatttgagtgctgcatgcattaggtatttgtcctaatgctctccctccccttgtcccccatcccctgagaggccccagtgtgtgatgttcccttctctgtgtccatgtgttctcattgttcaactctcacttatgagtgaaaacatgtggtgtttggttttctgttcctgtgttagtttgctgagaataatggtttccagattcatccatgtccctgcaaaggacatgaattcattcttttttatggctgcatagtattccatggtgtatatgtgccatattttctatatccagtctatcattgatgggcatttgggttggttccaagcctttgcgattgtgaatagcactgcaataaacatacatgtgcatgtatctttacagtagaatgatttatcatcctttgggtgtttatacccagtaatgggattgttgggtcaaatggtatttctggttctagatcttgaggaatcgccacactgtcttccacaatggctgaactaatttacactgacacagtgtaaaagctttcctatttctcgacatcctctccagcatctgttgtttcctgactttttaatatttgccattctaactggcatgagatggtatctcactgcggttttgatttgcatttctctaacgaccagtgatgagctttttttttcatgtttgtaggctgcataaatgccttctttagagaagtgtctgttcatatccttcacccactttttgatggggttgtttttttcttgtaaatttgtttaagttccttgtagattctggatgttagacctttgtcagatggagagattgcaaaaaatttctcccattctgtaggttgcctgttcactctgatgatagtttcttttactatgcagaagctccttagtttaagtagatcccatctgtcaattttggcttttgttgccattgcttttggtgtttttgtcatgaagtctttgcccatgcctatgtcctgaggggtattgcctaggttttcttctagggtttttatggttttaggttttatgtttaaatctttaggcaatcttgtgttaatttttgtataaggtgtaaggaaggggtccagtttcagttttctgcatatgcttagccagtgtccccagcaccacttattaaatagggaatcctctccccattgcttatttttgtcaggtttgttgaagatcagatggttgtagatgtgtggtgttatttctgtggcctttgttctgttccattggtctatatatctgttttggtaccagtaccatgctgttttggttgctgtagccttgtagtatagtttgaagccagggagtgtgatgcctccagctttgttctttttgcttaggattgtcatggctatacaggctcttttctggttccacataaaatttaaagtagttttttctagttctgtgaagaaaatcaatggtagcttgatgggaatagcactgaatctataaattactttgggcagtatggccattttcacaatatcgattcttcctatccatgaggatggaatgtttttccatttgtttgtgtcctctcttatttccttgagcagtggtttgtagttcttgaagaggttcttcgcgtcccttgtaagttgtattcctaggtattttatttcctttgtagcaattgtgaatgagagttcactcatgatttggctctcttttcatctgttattggtgtttaggaatgcttgtgggttttgcacattgattttgtatcctgagactttgctgaaggttcttatcagcttaaggagattttgggcagagatgatggggttttctaaatatataatcacatcatctggaaatagacaatttgacttcccctcttcctatctgaatactctttctttctcttgcctgattgccctagccagaacttccaatactatgttgaataggagtggtgagagagggcatccttgtcttgtgccagttttcaaagggaatgcttccagcttttgcccattcagtatgatattggctgtgggtttgtcataaataggcctcattattttgacatatgttccataaatccctagtttattgagtgtttttagcatgaagggatgttgaattttattgaaggccttttctgcatctattgagataatcatgtggtttttgtcattggttctgtttatgtgatggattatgtttattgatttgcatatgtttgaaccagctttgtatcccagggatgaagccgacttgatcgtggtggataagccttttgatgtgctcttggattcgctttgccagtattttattgaggattttcgcagcgatgttcatcagggatattggcttgaaattttatttttttgttgtgtctctgctaggttttggtatcaggatgatgctggccttataaaattagggaggattccctcttttcctattgtttggagtagcttcaggaggaatggaaccaactcctctttgtacctctggtagaatttggctgtgaatccatctggtcctgggctttttttggttcgtaggctattaattactgcctcaatttcagaacttgttattagtgtatatggggattcaacttcttcctgggttagtcttgggagggtgtatgtatccaagaatttatccatttcttctacattttctagtttacttgcatagaggttttcatagcagtctctgatggtagtttttatttctgtgggatcagtggtgatattccctttatcatttttttattgtgtctatttgattcttctctcttttcttctttattagtctggctagcagtctatctattttgttaatcttttcaaaaaaccagctcctggattcattgattttttgaagggtttttcatgtctctgtctccttcagttctgctgttagtgtgttggttttagatctttcctgctttctgatgtgggcattatagtgctataaatttccctcttaacactgctttagctgtgtcccacagattctggcatgttgtgtctttattctcattggtttcaaagaacttatttatttctgcctcaattttgttatttacccagtagtcattcaggagcaggttgttcagtttccatgtagttgtgtggttttgaatgagtttcttaatcctgagttctaatttgattgcactatggtctgagagactgtttgttacgatttccattcttttgcaattgctgaggagtgttttactttcaattacgtggtcaattttagaataactgctacgtggtgaagagaagaatgtatattctattgatttggggtagagagttctgtagatgtctattaggtctgcttggtccagagctgagtttaagtcctgaatatccttgttaattttctgtctcattgttctgcctaatactgacagtggggtgttaaagtctcctactattattgtgttggagactaagtctctttgtaggtctctaagaacttgctttatgaatctgggtgctcccgtactgggtacatatatatttaggaaagttagctcttcttgttgcattgatccctttaccattatgtaatgcccttctttgtctttttttcatctttgttggtttaaagtctgttttatcagagactaggattgcaacacctgctgtttttatttatttatttatttatttatttttatttttttagctttccatttgcttggtaactattcctccatctctttattttgagcgtatgtgtgtctttgcatgtgagttgggtctcctaaatacagcacaccaatgggtcttgactctatccaatttgccagtctgtgtcttttaattggggcatttagtccatttacttaaaggtaatatcattatgtgtgaatttgttcctgtcatcatgacgctagctgtttattttgcacattagttgatgcagtttcttcatagtgtcaatggtctttacatttcggtttgtttttgcagtggctggtaccagttttttctttccatgtttagtgcttccttcaggaactcttgtaaggcaggcctggtgttgacaaaatccctcagcatttgcttgtctataaaggatttttttctccttaacttatgaagcttagtttggctggatataaaattatgggtcgaaaattcttttctttaagaatgttgaatattggcccccactctcttctggcttgtagggtttctgcagagagatccactgttagtctaatgggcttccctttgtaggtaacctgacctttctctctggctgtgcttaacactttttccttgttttcaaccttggaaaatctgatgattatgtgtcttggggttgctcttctcgaggagtatctttttggtgttctctatatttcctgaatttgaatgttgacctgtcttgttaggctgtggaagttctcttggaaaatatcctgaagtatgtttcttggaaaatatcctgaagtatgtttggaaaatatcctgaagtatactttccaacttggttccgttctccccatcactttcaggtacactaatcaatcataggtcttttcacatagtcccatatttcttggaggctttatttgttccttttcattcttttttctctaatcttgtcttcacgctttatttcattaagctgatcttcaatctctgatatcctttcttctgcttgatcgatttggctattgatacttgtgtatgccttacgaagttctcatgctgtgtttttcagctccatcagatcatttatgttcttctctaaactgcttattctagttagcaattcctctaaccttttatcaaggttcttagcttccttgcattggtttagaacatgctcctttagctcagtggagtttgatattacccaccttctgaagcctacttctgtcgatttgtcaaactcattctccatccagttttgtgcccttgctggagaggagttgcaatcatttggaggagaagaggcattctgttttttggaattttcaacatttttgcactgttttttcctcatcttcgtggatttatctacctttgatttttgatactaatgacctttgggtggggtttttctgtgggcatcttttttgttgatgttgatgttattgctttctgtttgttagttttccttctaacagtcaggcccctcttctgcaggtctgctggagtttgctggaggtccactccagaccctgtttgcctgggtatcaccagtggaggctgcagaacagcaaatattgctgcctgctccttcctttagtagcttcgtcccagagtggcatctgccagaagtcagctggagctcttctgtatgaggtgtctgttgaaccctgctggaaggtgtctccctgtcaggaggcatgagggtcagggacccccttaaggagacagtctgtctcttagcagagcttggacactgtgctgggagatccgctgctctcttcagagctgacaggcagaacgtttaagtctgctgaagctggatccacagccgccccttcccccaggtgctctgtcccaggcagatgggaattttacctatcagcccctggctggggctgctgcctttctttcagagatgccctgcccagagaggaggaatctggagaggcagtctggccccaggtactttgccatgctgcggtgagttctgcagtcagagttaacactgtaaggggaaaagcacctactcaagcctcagtaatggtggacgcccctcccccaacaagctcaattgtcccaggttgacttcatactgctgtgctggcagcgagaatttcaagccagtggattttagcttgctggactttgtgggaatgggacccgctgtgcgagaagacttggcttcctggcttcagccccctttccaggggagtgagtggttctgtctcactggggttccaggcactgctggtgtatgaaaaaaaactcctgcagctgtttatctgcccagagagttgcctagttttgtgcttgaaacccagggccctggtagtgtaggcacacgagggaatctcctggtctgtgggtggcaaaaaccatggaaaaagcatagtatctgggctggatagcacagtccctcacagtagagtccctcagggcttcccttggctaggagagggaggtccatgaccccttgcacttcctgggtgaggcgataccccaccctgcttctgttccccctccatgggctgcacccactgtctaaccagtcccagtgagataaaccgggcacctcagttggaaatgcaataatcacccaccttctgcgttggtcttgctgggagctgcagactgaagctgttcctattcagccatcttgccaactctcttgtattagttttctattgctgccataacgaatagtctccatcttagcagctaaaaacaacacccatttatcaactcacagttctgcaggtcagagtctcgtaggcttggctagtttctctgtcctgagtctcatcagattgacagcaaggggctgacaaggctgtattcccttttgaaaattttggagatggatgtgtgtctttgttcattcaggttattcactgaattcagttcctgtggttgtagcaatgagatctccgtttcctttctggcggtcagcccagggtagcctttgctcctagatgctgcttgcatccctcaatctttccaggtggcccctccacccacagcaggtagggtccaggtccctctcttgctttagatctttcctccttactcttctcactccagccacatttctctcactccagccagagacatttctctaattttagatgggttccactccctggtttaaggtctgctatcttaattgcatctgccaaatctcatcatagcagtacccaggttaatatttgattgaattactgggagataggaatagtgagggactaccacagcctaggaagtcttgaatgatatgattcctgtgacttctctgacttcatgtcatccctctttcccttttaatatctagactccaagtcagtatggcctccttgctgttttctccaattgaccaaacctatatccacttgagggccttgacacctcacatttcctctgccagaacacccttgcccgaggcctttccatggcccactctctcactaagttggatgagtcctctgggaagcagactccaaggcagtattaggagagctcaagatttattgggggtaaggcctgtgaaagataaaggagagaagggacaggagtgggaaggctgggccttcagaccacagtgcaggtctgacatccttgcagggataggaggggataggaggattggatgaagagcctcagatttccatgcatactgcactgagaaagtgtcagccagcccagcagggagcttttgccacagagattgcctgtggaagggttccacgctgcccgggaaatggcaagcctttgtatcattgtgctccgtcattggctgaggattatccaggaagcatgtgacctccactcaaaagctgagggagaaacaaccaaatgtccatcaacagatgagtggatggacaaaacgtagtatgcacattcagtggaatattatccagccatgaaaaggaatgaagtattgacacacactaccacatggatgaacattgaaaacactgtgctcagtgaaataaaacagtcacaaaaggacaaatattgtatgcttctacttatggtaaatatttagaacaggtaaatccatagagacaaaatatagattagaaggtccaaggcttgcagaaggaagaaatgaggagttatcatttaacgggtacagtttctgtttgaggtgttaacaatttttgaaactggatagtggtggtgatagttgcacagaattgtgaatataattaacaccactttaaaatcttaaaaatggttgaagtggcaaattttatgctaaatacattttactacagtaaaaaacaaaacaaaacaaaaaaacagaccgaggcagatcctgaaagcaccagcagctagaagctgtcagcaaatagcaccttttgtgggaagcacatctccatggctgccatgctcactgcctcgggtctctgccgaaatggcaactgagcagtgagtcccttaatgactaaattacatccaagaggaaggcctatccttctcagtcaagttaatttttctccattacacttaaagccttctgacttatatatttacctgtttattttctgcttccccaatagaccataaacaccatgagatcagagcatccatctgtagactcacagagcaagaagcgtgactgccccaatcttgaatggatgagtaag

SEQ ID NO: 59 - RNA, 2861 nt, Homo sapiens
  source 1..2861: mol_type = unassigned RNA
ctcgcaagacctggttgtttaaaagtgggcggcaccttccccttcgctcttcctcctgctcctgccctgatgtgtctggatggtgacagatgggaatcttctccagtgaccagaatccatgccttaccttgggagcccaagctgacttctcaagctctggggatgcctcagtgatctctgatgtggaattagtggaggctggccgttgctcacctctacacagcctgctggtcgttagcgagcaggagttctgatgttcctctcatcttggatgtctttcctaggagaatatttctcacgtgtttgacagaaaacattatctgtccatcatttccatggattcctgggtctcacgggcagaaggacaaggcagatatgaggcggatctggtaaaaccataaacaccatgagatcagagcatccatctgtagactcacagagcaagaagcgtgactgccccaatcttgaatggatgagtaagatgaaaatcaatttataaacatcttgtgacaagctggaattcagttatagatcaattacagattgctcactggacaatcaaggacattattagaaaacggtttccaccactagaaattcatctacattgacatccacctaaatatccagaaggtcatccccactttcgtcgctctccacagtcagctgccgggtccaccacttcttaacttcagaaccgcaggaagccgcgtcagtcatggggttcattttgcacacgacagatttcatggttgtccttcctccaaaccgaggaacctagatcttctctgatctctgaaaaataagttcctggatatgaaaggggatactttccttggtggtagtcccagtgaagacccactgaacatcgtcagcctcaaaatgtaagctgtgctgtcatctgctttgtggaacacatatgctacacatcttctcctttcaattgggctgaagttggggcacagttaaagagcattataaagctcctgctttagcatgaacttagttatctatagaaaaaaacatgtacaaggacactgggctgtggggagcattgctctccctccattcctgagtgctcttaatgggattatcctggctttccaaaaccctatagagaagaagctcatagaagaaaattctcttactgaatcagtaaaatatggaaaataatgggactgtttattaaaggattgaatgagagaacacaagtaatgcacttagaagagtgactggcatacagtgagtactacgtgctggcttctcaaccatgatagcctgaaatgtacccggaaaatgggggctaatgtaagtcatatttagagaagttattttgggggacaatgtttttgacaagactacatttttcattatgttgctgtctgtaaacaactgtagatgcagacttgcattttgaaaggcaagacccaaaacacacaaacagagtaaatacctttaaaatgcctcgatcgctggagaaatgcctggatggacctacctgagggcagacgccactgcccaaatttcctctggggtttccctgcgtcggctgcatcttgccgacttcctcctcgatccgacagcgtggggctcaggagctgctgctggctgcttgtctgcatgggaggagaagcacacagttaccgtctttttaaccctgcgctctcctccttttactataagaaaggaaaacacatggcaggaggacaagtgggaagaacaacatggactcagagtgtccacaatggggaaaagtgaaaatgggtataaagggtcacagaagaaaaagagccaccactgcacgccctgctgaaggagacaccactcttcctgggacatcttgtcctgggtaacccccactgcttcgttcccctccatgcctgagaatgtcattgtttcccttcatatcttcgctggcaaaccagggggtaaggaaggggaactgcaggaaggaaggtggggcagtggggacgggctgggcgggggcagtctactgtcaccagcctggagatccacacttagagcttcagggaaagagttctctgcaggcaaagctgtatgatgggccctggtggaggatggagctgataacaatctaagctacagtgaaagaaagagagatgccaagacttcccacacatttcacctgttctcaggttcacctgtcacctctccctttcctaacaaaagccttggtcttcacatctctttagtatccttttttttttttttttgtcacctctttctttcttaggctttttcatctaaaaacagagggctctgaaatcatcactttccccaacctcattagccctgtctcactgtctgtcctgacttcagcctgcctccctctcccatgcgagacattgccaaccatcttcagggtgaccagtggcaaggccactgcagatgcctccccttgccccttggttgactaactccgcagaagtccctgcagtcggtggcagatgatttctaaaggctggggagcccaaaacatgagatctactgaaaactgagctatggcaccagtgggcctcctggagcaccctgctgggccgtggagccttaggcttccaaaggtactctggcccctggctgcgttgctgcacaccatggagcctggtgctgctgggggctctgcacagagcacgggatcagctccacgttgttaacctcaaatcgtaattgctgcctcataaaggggcaggtcctcccagccagctgcttctcacagcacaggactctccaggacgctgataactccttgtacttttttatatagacacatacatagagtttgaaaatgaaaatatgctgtgacttaataaatagaaactcctcaaaaaaaaaaaaaaaaa

(19) United States
(12) Patent Application Publication
(10) Pub. No.: US 2024/0376440 A1
(43) Pub. Date: Nov. 14, 2024

(54) ENGINEERED PRODUCER CELL LINES AND METHODS OF MAKING AND USING THE SAME

(71) Applicant: Ultragenyx Pharmaceutical Inc., Novato, CA (US)

(72) Inventors: Aubrey R. Tiernan, Somerville, MA (US); Nicholas Richards, Arlington, MA (US)

(21) Appl. No.: 18/659,358

(22) Filed: May 9, 2024

Related U.S. Application Data
(62) Division of application No. 16/844,475, filed on Apr. 9, 2020, now Pat. No. 12,018,288.
(60) Provisional application No. 62/979,483, filed on Feb. 21, 2020.
(60) Provisional application No. 62/839,207, filed on Apr. 26, 2019.
(60) Provisional application No. 62/833,548, filed on Apr. 12, 2019.

(51) Int. Cl. C12N 7/00; C12N 15/11; C12N 15/86; C12Q 1/6869

(52) U.S. Cl. CPC C12N 7/00; C12N 15/11; C12N 15/86; C12Q 1/6869; C12N 2310/14; C12N 2310/315; C12N 2310/321; C12N 2750/14143; C12N 2750/14152

(57) ABSTRACT

This application relates to recombinant adeno-associated virus (rAAV) packaging and/or producer cell lines which have been engineered to reduce expression and/or activity of one or more genes and/or proteins to increase rAAV titers. The methods of generating the engineered cell lines have also been described herein.

### CROSS-REFERENCE TO RELATED APPLICATIONS

This application is a divisional of U.S. patent application Ser. No. 16/844,475, filed on Apr. 9, 2020, which claims the benefit of and priority to U.S. Provisional Patent Application No. 62/833,548, filed Apr. 12, 2019; to U.S. Provisional Patent Application No. 62/839,207, filed Apr. 26, 2019; and to U.S. Provisional Patent Application No. 62/979,483, filed Feb. 21, 2020, the entire disclosure of each of which is incorporated herein by reference in its entireties for all purposes.

### SEQUENCE LISTING

This application contains a Sequence Listing which has been submitted electronically in XML format and is hereby incorporated by reference in its entirety. The XML copy, created on Jul. 23, 2024, is named ULTA-015USD1_SL.xml and is 130.8 kilobytes in size.

### FIELD OF THE DISCLOSURE

This application relates generally to engineered producer and/or packaging cell lines and methods of generating the engineered producer and/or packaging cell lines for increasing recombinant adeno-associated virus (rAAV) titer.

### BACKGROUND

rAAV-based vectors are one of the most promising vehicles for human gene therapy. rAAV vectors are under consideration for a wide variety of gene therapy applications. In particular, rAAV vectors can deliver therapeutic genes to dividing and nondividing cells, and these genes can persist for extended periods without integrating into the genome of the targeted cell. Although systems for producing rAAV have evolved over the last two decades, several issues remain to be solved. One limitation of rAAV production systems is the low titer yield of rAAV particles. Pharmaceutical development of rAAV-based gene products at preclinical stage require large amounts of rAAV vectors for studies in larger species to enable complete toxicology and biodistribution studies that are helpful in predicting dosages in humans. Furthermore, because current rAAV production systems result in low titer yields, manufacturing sufficient levels of rAAV for use in human trials and commercial applications is challenging. Researchers have explored numerous ways to generate adequately high titers of rAAV particles, but there is still a great need for addressing this issue. In particular, there is a need for efficient cell lines that are able to produce high quality rAAV with high titer yields. Production of high titer rAAV by the engineered cell lines described herein expedites the application of this vector system for gene therapy use in vivo.

### SUMMARY

The present disclosure addresses the need for obtaining improved rAAV titers for gene therapy applications by providing rAAV packaging and/or producer cell lines comprising cells in which one or more genes and/or proteins have been modified. Also described herein are methods of identifying one or more genes and/or proteins that are relevant to the production of rAAV, and methods of generating engineered rAAV packaging and/or producer cell lines.

Described herein are compositions and methods of generating rAAV packaging and/or producer cell lines comprising cells that can produce a higher titer of rAAV compared to control parental cells. More specifically, provided herein are rAAV packaging and/or producer cell lines comprising cells in which expression of one or more genes and/or proteins is modulated resulting in a higher rAAV titer compared to control parental cells. In one aspect, the present disclosure provides rAAV packaging and/or producer cell lines comprising cells in which expression of one or more genes and/or proteins is reduced compared to control parental cells. For example, expression of ATP5EP2 (ATP Synthase F1 Subunit Epsilon Pseudogene 2), LINC00319 (Long Intergenic Non-Protein Coding RNA 319), CYP3A7 (Cytochrome P450 Family 3 Subfamily A Member 7), ABCA10 (ATP Binding Cassette Subfamily A Member 10), NOG (Noggin), RGMA (Repulsive Guidance Molecule BMP Co-Receptor A), SPANXN3 (SPANX Family Member N3), PGA5 (Pepsinogen A5), MYRIP (Myosin VIIA And Rab Interacting Protein), KCNN2 (Potassium Calcium-Activated Channel Subfamily N Member 2), and/or NALCN-AS1 (NALCN Antisense RNA 1) is reduced compared to control parental cells.

In some embodiments, the present disclosure provides rAAV packaging and/or producer cell lines comprising cells in which expression of KCNN2, LINC00319, RGMA, and SPANXN3 is reduced compared to control parental cells.

In certain embodiments, the present disclosure provides a rAAV packaging and/or producer cell line comprising cells which have been engineered to reduce the expression and/or activity of a gene product expressed from ATP5EP2, LINC00319, CYP3A7, ABCA10, NOG, RGMA, SPANXN3, PGA5, MYRIP, KCNN2, and/or NALCN-AS1 as compared to corresponding unmodified parental cells. In certain embodiments, the present disclosure provides a rAAV packaging and/or producer cell line that exhibits reduced expression and/or activity of a polypeptide or a polyribonucleotide expressed from at least one of ATP5EP2, LINC00319, CYP3A7, ABCA10, NOG, RGMA, SPANXN3, PGA5, MYRIP, KCNN2, and NALCN-AS1 as compared to a corresponding parental cell line.

In one aspect, the present disclosure provides a rAAV packaging and/or producer cell line in which expression of one or more genes is reduced using a nuclease, a double stranded RNA (dsRNA), a small interfering RNA (siRNA), a small hairpin RNA (shRNA), a microRNA (miRNA), or an antisense RNA oligonucleotide (ASO).

In certain embodiments, the expression of one or more genes is reduced with an siRNA comprising a nucleotide sequence selected from any one of sequences SEQ ID NOs: 1-11. For example, in some embodiments, expression of ATP5EP2 is reduced, and the siRNA comprises the nucleotide sequence of SEQ ID NO: 1 in the sense strand and the nucleotide sequence of SEQ ID NO: 32 in the anti-sense strand. In some embodiments, expression of LINC00319 is reduced, and the siRNA comprises the nucleotide sequence of SEQ ID NO: 2 in the sense strand and the nucleotide sequence of SEQ ID NO: 33 in the anti-sense strand. In some embodiments, expression of CYP3A7 is reduced, and the siRNA comprises the nucleotide sequence of SEQ ID NO: 3 in the sense strand and the nucleotide sequence of SEQ ID NO: 34 in the anti-sense strand. In some embodiments, expression of NOG is reduced, and the siRNA comprises the nucleotide sequence of SEQ ID NO: 4 in the sense strand and the nucleotide sequence of SEQ ID NO: 35 in the anti-sense strand. In some embodiments, expression of SPANXN3 is reduced, and the siRNA comprises the nucleotide sequence of SEQ ID NO: 5 in the sense strand and the nucleotide sequence of SEQ ID NO: 36 in the anti-sense strand. In some embodiments, expression of MYRIP is reduced, and the siRNA comprises the nucleotide sequence of SEQ ID NO: 6 in the sense strand and the nucleotide sequence of SEQ ID NO: 37 in the anti-sense strand. In some embodiments, expression of KCNN2 is reduced, and the siRNA comprises the nucleotide sequence of SEQ ID NO: 7 in the sense strand and the nucleotide sequence of SEQ ID NO: 38 in the anti-sense strand. In some embodiments, expression of NALCN-AS1 is reduced, and the siRNA comprises the nucleotide sequence of SEQ ID NO: 8 in the sense strand and the nucleotide sequence of SEQ ID NO: 39 in the anti-sense strand. In some embodiments, expression of RGMA is reduced, and the siRNA comprises the nucleotide sequence of SEQ ID NO: 9 in the sense strand and the nucleotide sequence of SEQ ID NO: 40 in the anti-sense strand. In some embodiments, expression of PGA5 is reduced, and the siRNA comprises the sequence of SEQ ID NO: 10 in the sense strand and the sequence of SEQ ID NO: 41 in the anti-sense strand. In some embodiments, expression of ABCA10 is reduced, and the siRNA comprises the sequence of SEQ ID NO: 11 in the sense strand and the sequence of SEQ ID NO: 42 in the anti-sense strand.

In certain embodiments, the nuclease used to reduce expression of one or more genes is selected from the group consisting of a Zinc Finger nuclease (ZFN), a meganuclease, a transcription activator-like effector nuclease (TALEN), or a clustered regularly interspaced short palindromic repeats (CRISPR) associated protein.

In certain embodiments, the expression of one or more genes is reduced using CRISPR genome editing. In some embodiments, a guide RNA pair is used to target a gene to reduce and/or eliminate expression of that gene. In certain embodiments, the expression of one or more genes is reduced using a guide RNA pair, wherein each guide RNA: (a) comprises a sequence selected from the nucleotide sequences of SEQ ID NOs: 12-15 and/or (b) targets a target DNA sequence selected from any one of the nucleotide sequences of SEQ ID NO: 16-31. For example, in some embodiments, the gRNA pair is used to target KCNN2 and comprises a first gRNA molecule comprising the sequence of SEQ ID NO: 12 and a second gRNA molecule comprising the sequence of SEQ ID NO: 13. In some embodiments, the gRNA pair is used to target KCNN2 and comprises a first gRNA molecule comprising the sequence of SEQ ID NO: 14 and a second gRNA molecule comprising the sequence of SEQ ID NO: 15. In some embodiments, each gRNA molecule is a 2′ O-methyl analog comprising 3′ phosphorothioate internucleotide linkages in the terminal three nucleotides on either or both its 5′ and 3′ ends.

In certain embodiments, one guide RNA pair is used to reduce expression of one gene. In certain other embodiments, multiple guide RNA pairs are used to reduce expression of one or more genes. In certain embodiments, the gene expression of one or more genes and/or the activity of one or more genes and/or proteins is reduced and/or eliminated in a rAAV packaging and/or producer cell line compared to a control parental cell line. In certain embodiments, the gene expression and/or activity is eliminated in the rAAV packaging and/or producer cells compared to control parental cells.

In some embodiments described herein, the rAAV packaging and/or producer cell line is a eukaryotic cell line. In certain embodiments, the rAAV packaging and/or producer cell line is a human cell line. In certain embodiments, the rAAV packaging and/or producer cell line is an insect cell line. In certain embodiments, the rAAV packaging and/or producer cell line is a HeLa cell line. In certain other embodiments, the rAAV packaging and/or producer cell line is a human embryonic kidney (HEK) 293 cell line.

In some embodiments described herein, the rAAV packaging and/or producer cell line of the present disclosure produces a higher rAAV titer than a control parental cell line. In certain embodiments, the titer of rAAV produced from cells of the rAAV producer cell line of the present disclosure is increased about 1.5 to about 7 fold compared to the titer of rAAV produced from a cell line comprising the control parental cells. Also described herein are lysate of the engineered cell lines. In certain embodiments, higher titer rAAV is harvested from a lysate. Also described herein are cell culture supernatants from engineered cell lines. In certain embodiments, higher titer rAAV is harvested from a cell culture supernatant.

Also described herein is a method of generating a producer cell line where the method includes delivering a rAAV vector to cells of a packaging cell line in which the expression of ATP5EP2, LINC00319, CYP3A7, ABCA10, NOG, RGMA, SPANXN3, PGA5, MYRIP, KCNN2, and/or NALCN-AS1 is reduced compared to control parental cells. In certain embodiments, the present disclosure provides a method of generating a producer cell line, where the method includes delivering a rAAV vector to cells of a packaging cell line in which the expression of KCNN2, LINC00319, RGMA, and SPANXN3 is reduced compared to control parental cell.

Also described herein is a method of producing rAAV by infecting the cells of a producer cell line, generated by a packaging cell line, with a helper virus, wherein the expression of ATP5EP2, LINC00319, CYP3A7, ABCA10, NOG, RGMA, SPANXN3, PGA5, MYRIP, KCNN2, and/or NALCN-AS1 is reduced in the packaging cell line compared to control parental cells. In certain embodiments, the expression of KCNN2, LINC00319, RGMA, and SPANXN3 is reduced in the packaging cell line compared to control parental cells.

In one aspect, the present disclosure provides a method of producing rAAV, by infecting the cells of a producer cell line with a helper virus, wherein the expression of ATP5EP2, LINC00319, CYP3A7, ABCA10, NOG, RGMA, SPANXN3, PGA5, MYRIP, KCNN2, and/or NALCN-AS1 is reduced in the producer cell line compared to control parental cells. In certain embodiments, the present disclosure provides a method of producing rAAV, by infecting the cells of a producer cell line with a helper virus, wherein the expression of KCNN2, LINC00319, RGMA, and SPANXN3 is reduced in the producer cell line compared to control parental cells.

Also described herein is a method of harvesting rAAV from a producer cell line in which the expression of ATPSEP2, LINC00319, CYP3A7, ABCA10, NOG, RGMA, SPANXN3, PGA5, MYRIP, KCNN2, and/or NALCN-AS1 is reduced compared to control parental cell line. Also described is a method of harvesting rAAV from a producer cell line in which the expression of KCNN2, LINC00319, RGMA, and SPANXN3 is reduced compared to control parental cell line. In certain embodiments, the production of rAAV from a producer cell line of the present disclosure is enhanced compared to a control parental cell line.

Also described herein is a method of identifying one or more genes relevant to the production of rAAV, where the method includes i.) adding one or more supplements that increase the rAAV titer in a cell line; ii.) measuring the global gene expression across the transcriptome in supplemented and non-supplemented cell lines; iii.) obtaining a list of genes that are differentially expressed between supplemented and non-supplemented cell lines; and iv.) identifying one or more genes that are relevant to the production of rAAV. In some embodiments, the one or more identified gene(s) is responsible for reducing the production of rAAV.

Also described herein is a method of producing a rAAV packaging and/or producer cell line to promote increased production of rAAV. In some embodiments, rAAV production is increased by modulating the expression of one or more genes and/or proteins identified from a list of genes that are differentially expressed between supplemented and non-supplemented rAAV producer cell lines. In certain embodiments, rAAV titer is increased by modulating the expression of one or more genes and/or proteins identified from a list of genes that are differentially expressed between supplemented and non-supplemented rAAV producer cell line. In some embodiments, the modulation of one or more genes and/or proteins increases rAAV titer at least 1.5 fold compared to rAAV titer of a cell line without the modulation. In certain embodiments, modulating the expression is reduction of expression of one or more genes. In certain embodiments, modulating the expression comprises reduction of expression of one or more proteins. In certain embodiments, modulating the expression is elimination of expression of one or more genes. In certain embodiments, modulating the expression comprises elimination of expression of one or more proteins.

In some embodiments, the rAAV packaging and/or producer cell line is a eukaryotic cell line. In certain embodiments, the cell line is a human cell line. In certain embodiments, the cell line is an insect cell line. In certain embodiments, the cell line is a HeLa cell line. In certain embodiments, the cell line is a human embryonic kidney (HEK) 293 cell line.

Also described herein is a recombinant adeno-associated virus (rAAV) packaging and/or producer cell line comprising cells which have been engineered to reduce the expression and/or activity of a gene product expressed from ATP5EP2, LINC00319, CYP3A7, ABCA10, NOG, RGMA, SPANXN3, PGA5, MYRIP, KCNN2, and/or NALCN-AS1 as compared to corresponding unmodified parental cells.

In some embodiments, the expression and/or activity of a gene product expressed from ATPSEP2, LINC00319, CYP3A7, ABCA10, NOG, RGMA, SPANXN3, PGA5, MYRIP, KCNN2, and/or NALCN-AS1 is reduced indefinitely or permanently.

In some embodiments, the cell line has been engineered to comprise a gene disruption or a partial or complete gene deletion in at least one of ATP5EP2, LINC00319, CYP3A7, ABCA10, NOG, RGMA, SPANXN3, PGA5, MYRIP, KCNN2, and/or NALCN-AS1.

In some embodiments, the cell line has been engineered to comprise a gene disruption in at least one of ATP5EP2, LINC00319, CYP3A7, ABCA10, NOG, RGMA, SPANXN3, PGA5, MYRIP, KCNN2, and/or NALCN-AS1.

In some embodiments, the cell line has been engineered to comprise a gene disruption in at least two genes selected from ATP5EP2, LINC00319, CYP3A7, ABCA10, NOG, RGMA, SPANXN3, PGA5, MYRIP, KCNN2, and NALCN-AS1.

In some embodiments, the cell line has been engineered to comprise a partial or complete gene deletion in at least one of ATP5EP2, LINC00319, CYP3A7, ABCA10, NOG, RGMA, SPANXN3, PGA5, MYRIP, KCNN2, and/or NALCN-AS1.

In some embodiments, the cell line has been engineered to comprise a partial or complete gene deletion in at least two genes selected from ATPSEP2, LINC00319, CYP3A7, ABCA10, NOG, RGMA, SPANXN3, PGA5, MYRIP, KCNN2, and NALCN-AS1.

Also provided is a packaging and/or producer cell line, wherein said cell line exhibits reduced expression and/or activity of a polypeptide or polyribonucleotide expressed from at least one of ATPSEP2, LINC00319, CYP3A7, ABCA10, NOG, RGMA, SPANXN3, PGA5, MYRIP, KCNN2, and NALCN-AS1 as compared to a corresponding parental cell line.

Other features and advantages of the disclosure will be apparent from the following detailed description and claims.

Unless noted to the contrary, all publications, references, patents and/or patent applications reference herein are hereby incorporated by reference in their entirety for all purposes.

### BRIEF DESCRIPTION OF THE DRAWINGS

The disclosure can be more completely understood with reference to the following.

FIG. 1 is a schematic showing methods of generating rAAV packaging and producer cells described herein.

FIGS. 2A-2D show experimental data generated from the optimization and development of HPRT1 siRNA knockdown experiments. FIGS. 2A-2B show percent knockdown (FIG. 2A) and protein expression (FIG. 2B) data generated from HPRT1 siRNA knockdown experiments performed in 24 wells. FIGS. 2C-2D show percent knockdown (FIG. 2C) and protein expression (FIG. 2D) data generated from HPRT1 siRNA knockdown experiments performed in 6 wells.

FIGS. 3A-3B show the log fold change values in gene expression obtained from bioinformatic analysis of RNA-Seq data for PGA5 (FIG. 3A) and SPANXN3 (FIG. 3B), represented as log fold change in gene expression in cells cultured in unsupplemented cell culture medium relative to uninfected cells (cells not infected with a helper virus), and log fold change in gene expression in cells cultured in supplemented cell culture medium relative to unsupplemented cell culture medium. FIGS. 3C-3D show RT-qPCR fold change values in the expression of PGA5 (FIG. 3C) and SPANXN3 (FIG. 3D) in cells cultured in unsupplemented and supplemented cell culture medium, relative to uninfected cells.

FIGS. 4A-4B show the fold change values in PGA5 (FIG. 4A) and SPANXN3 (FIG. 4B) expression in producer cell line clones cultured in unsupplemented cell culture medium and supplemented cell culture medium relative to uninfected cells (cells not infected with a helper virus), as determined from RT-qPCR. 21C5, 3C6, 2B6 represent different clones of the HeLa producer cell line. FIGS. 4C-4D show relative fold increase in PGA5 (FIG. 4C) and SPANXN3 (FIG. 4D) expression in producer cell line clones 21C5, 3C6, 2B6 cultured in supplemented cell culture medium compared to the clones cultured in non-supplemented cell culture medium.

FIGS. 5A-5F show the effect of reducing expression of individual genes in different producer cell lines on rAAV titers. The figures show the titers of produced rAAV in genome copies (GC) per milliliters (mL) for producer cell line #1 (FIG. 5A), producer cell line #2 (FIG. 5B), and producer cell line #3 (FIG. 5C). FIGS. 5D-5F show the fold change in titers of rAAV produced from producer cell line #1 (FIG. 5D), producer cell line #2 (FIG. 5E), and producer cell line #3 (FIG. 5F). FIGS. 5A-5B show the average across 3 biological replicates. FIGS. 5C-5F show the average across 4 biological replicates.

FIG. 6 is an illustrative flow-chart showing an exemplary gene filtering methodology.

FIG. 7A shows the 24 deep well titers of the top 19 2H5 knockout clones. Titer is reported as genome copies per mL. The control sample is unmodified 2H5. FIG. 7B shows the fold change in titer compared to the 2H5 control. 2H5 titer was set to 1 and other titers are displayed as the fold increase above the 2H5 control. FIG. 7C shows the 24 deep well titers of the top 19 7B12 knockout clones. Titer is reported as genome copies per mL. The control sample is unmodified 7B12. FIG. 7D shows the fold change in titer compared to the 7B12 control. 7B12 titer was set to 1 and other titers are displayed as the fold increase above the 7B12 control.

FIG. 8A shows the ambr® 15 titers of the top five 2H5 knockout clones. Titer is reported as genome copies per mL. The control sample is unmodified 2H5. FIG. 8B shows the fold change in titer compared to the 2H5 control. 2H5 titer was set to 1 and other titers are displayed as the fold increase above the 2H5 control. FIG. 8C shows the ambr® 15 titers of the top four 7B12 knockout clones. Titer is reported as genome copies per mL. The control sample is unmodified 7B12. FIG. 8D shows the fold change in titer compared to the 7B12 control. 7B12 titer was set to 1 and other titers are displayed as the fold increase above the 7B12 control.

FIGS. 9A-9B show the effect on rAAV titer generated by reducing expression of various gene combinations in two producer cell lines. The figures show fold change in rAAV titer compared to a control treated with missense siRNA. FIG. 9A shows the fold change in titer compared to the 2H5 missense control. 2H5 missense titer was set to 1 and other titers are displayed as the fold increase above the 2H5 missense control. FIG. 9B shows the fold change in titer compared to the 7B12 missense control. 7B12 missense titer was set to 1 and other titers are displayed as the fold increase above the 7B12 missense control.

### DETAILED DESCRIPTION OF THE DISCLOSURE

The present disclosure describes a recombinant adeno-associated virus (rAAV) packaging and/or producer cell line comprising cells in which expression of one or more genes and/or proteins is modulated. The modulation of gene expression results in an increased titer yield compared to a cell line in which expression of one or more genes and/or proteins in not modulated.

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8.

The following definitions are included for the purpose of understanding the present subject matter and for constructing the appended patent claims. Abbreviations used herein have their conventional meaning within the chemical and biological arts.

### Definitions

As used herein, “modulation” or “modulate” refers to the alteration of the regulation, expression or activity of a gene and/or protein. Modulation may be increasing, reducing (decreasing), or eliminating the expression and/or activity of one or more genes and/or proteins. In cases where multiple genes and/or proteins are modulated, all the expression and/or activity of genes and/or proteins may be increased, or all the expression and/or activity of genes and/or proteins may be decreased, or one or more genes and/or proteins may be increased and others of the genes and/or proteins may be decreased.

As used herein, the term “cell” refers to any cell or cells capable of producing a recombinant adeno-associated virus (rAAV). In some embodiments, the cell is a mammalian cell, for example, a HeLa cell, a COS cell, a HEK293 cell, a A549 cell, a BHK cell, or a Vero cell. In other embodiments, the cell is an insect cell, for example, a Sf9 cell, a Sf-21 cell, a Tn-368 cell, or a BTI-Tn-5B1-4 (High-Five) cell. The term “cell line” refers to a clonal population of cells able to continue to divide and not undergo senescence. Unless otherwise indicated, the terms “cell” or “cell line” are understood to include modified or engineered variants of the indicated cell or cell line.

As used herein, the term “engineered cell line” refer to cell lines that have been modified by one or more means to reduce the expression or other properties (e.g., biological activity) of one or more endogenously expressed genes and/or proteins (e.g., ATP5EP2, LINC00319, CYP3A7, ABCA10, NOG, RGMA, SPANXN3, PGA5, MYRIP, KCNN2, and/or NALCN-AS1) so as to augment the production of rAAV.

As used herein, the term “control parental cells” refer to cells that have not been modified by one or more means to reduce the expression or other properties (e.g., biological activity) of one or more endogenously expressed genes and/or proteins (e.g., ATP5EP2, LINC00319, CYP3A7, ABCA10, NOG, RGMA, SPANXN3, PGA5, MYRIP, KCNN2, and/or NALCN-AS1) so as to augment the production of rAAV.

As used herein, the term “control parental cell line” refers to a clonal population of control parental cells able to continue to divide and not undergo senescence.

“Lysis” refers to the breaking down of the cell, often by viral, enzymatic, or osmotic mechanisms that compromise its integrity. A “lysed cell” is a cell that has undergone substantial lysis. As used herein, the term “lysate” refers to a fluid containing the contents of lysed cells.

As used herein, the term “higher titer” signifies an increased titer in comparison to titer produced by an unmodified control parental cell line and/or control parental cell.

As used herein, the term “cell culture supernatant” refers to the cell culture media in which cells are suspended and/or cultured.

As used herein, the term “gene” refers to a transcription unit and regulatory regions that are adjacent (e.g., located upstream and downstream), and operably linked, to the transcription unit. A transcription unit is a series of nucleotides that are transcribed into an RNA molecule. A transcription unit may include a coding region. A “coding region” is a nucleotide sequence that encodes an unprocessed preRNA (i.e., an RNA molecule that includes both exons and introns) that is subsequently processed to an mRNA. A transcription unit may encode a non-coding RNA. A non-coding RNA is an RNA molecule that is not translated into a protein. Examples of non-coding RNAs include microRNA. The boundaries of a transcription unit are generally determined by an initiation site at its 5′ end and a transcription terminator at its 3′ end. A “regulatory region” is a nucleotide sequence that regulates expression of a transcription unit to which it is operably linked. Nonlimiting examples of regulatory sequences include promoters, enhancers, transcription initiation sites, translation start sites, translation stop sites, transcription terminators, and poly(A) signals. A regulatory region located upstream of a transcription unit may be referred to as a 5′ UTR, and a regulatory region located downstream of a transcription unit may be referred to as a 3′ UTR. A regulatory region may be transcribed and be part of an unprocessed preRNA.

In the context of this document, the term “target” or “target gene” refers to any gene, including protein-encoding genes and genes encoding non-coding RNAs (e.g., miRNA), that when modulated alters some aspect of virus production. Target genes include endogenous genes, viral genes, and transgenes.

With regard to gene designations, single genes have often been denoted by multiple symbols. In the context of this document, gene symbols, whether they be human or non-human, may be designated by either upper-case or lower case letters. Neither the use of one particular symbol nor the adoption of lower or upper case symbols is intended to limit the scope of the gene in the context of these disclosures. All gene identification numbers identified herein (GeneID) are derived from the National Center for Biotechnology Information “Entrez Gene” or KEGG web site unless identified otherwise.

The term “about” is used herein to mean approximately, in the region of, roughly or around. When the term “about” is used in conjunction with a numerical range, it modifies that range by extending, within permissible value ranges, the boundaries above and/or below the numerical values set forth.

As used in the present disclosure, whether in a transitional phrase or in the body of a claim, the terms “comprise(s)” and “comprising” are to be interpreted as having an open-ended meaning. That is, the terms are to be interpreted synonymously with the phrases “having at least” or “including at least.” When used in the context of a method, the term “comprising” means that the method includes at least the recited steps, but may include additional steps. When used in the context of a composition, the term “comprising” means that the composition includes at least the recited features or components, but may also include additional features or components.

For the purposes of promoting an understanding of the embodiments described herein, reference made to preferred embodiments and specific language is used to describe the same. The terminology used herein is for the purpose of describing particular embodiments only, and is not intended to limit the scope of the present disclosure. As used throughout this disclosure, the singular forms “a,” “an,” and “the” include plural reference unless the context clearly dictates otherwise. All percentages and ratios used herein, unless otherwise indicated, are by weight.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. In addition, the materials, methods and examples are illustrative only and are not intended to be limiting. All publications, patent applications, patents and other references mentioned herein are incorporated by reference.

### Adeno-Associated Virus (AAV)

AAV is a small, replication-defective, non-enveloped virus that infects humans and some other primate species. AAV is not known to cause disease and elicits a very mild immune response. Gene therapy vectors that utilize AAV can infect both dividing and quiescent cells and can persist in an extrachromosomal state without integrating into the genome of the host cell. These features make AAV an attractive viral vector for gene therapy. AAV includes numerous serologically distinguishable types including serotypes AAV-1 to AAV-12, as well as more than 100 serotypes from nonhuman primates (See, e.g., Srivastava, J. Cell Biochem., 105 (1): 17-24 (2008), and Gao et al., J. Virol., 78 (12), 6381-6388 (2004)). AAV is non-autonomously replicating, and has a life cycle with a latent phase and an infectious phase. In the latent phase, after a cell is infected with an AAV, the AAV site-specifically integrates into the host's genome as a provirus. The infectious phase does not occur unless the cell is also infected with a helper virus (for example, adenovirus (AV) or herpes simplex virus), which allows the AAV to replicate.

The wild-type AAV genome contains two 145 nucleotide inverted terminal repeats (ITRs), which contain signal sequences directing AAV replication, genome encapsidation and integration. In addition to the ITRs, three AAV promoters, p5, p19, and p40, drive expression of two open reading frames encoding rep and cap genes. Two rep promoters, coupled with differential splicing of the single AAV intron, result in the production of four rep proteins (Rep 78, Rep 68, Rep 52, and Rep 40) from the rep gene. Rep proteins are responsible for genomic replication. The cap gene is expressed from the p40 promoter, and encodes three capsid proteins (VP1, VP2, and VP3) which are splice variants of the cap gene. These proteins form the capsid of the AAV particle.

Because the cis-acting signals for replication, encapsidation, and integration are contained within the ITRs, some or all of the 4.3 kb internal genome may be replaced with foreign DNA, for example, an expression cassette for an exogenous protein of interest. In this case, the rep and cap proteins are provided in trans on, for example, a plasmid. In order to produce an AAV vector, a cell line permissive of AAV replication must express the rep and cap genes, the ITR-flanked expression cassette, and helper functions provided by a helper virus, for example AV genes Ela, Elb55K, E2a, E4orf6, and VA (Weitzman et al., Adeno-associated virus biology. Adeno-Associated Virus: Methods and Protocols, pp. 1-23, 2011). Production of AAV vector can also result in the production of helper virus particles, which must be removed or inactivated prior to use of the AAV vector. Numerous cell types are suitable for producing AAV vectors, including HEK293 cells, COS cells, HeLa cells, BHK cells, Vero cells, as well as insect cells (See e.g. U.S. Pat. Nos. 6,156,303, 5,387,484, 5,741,683, 5,691,176, 5,688,676, 8,163,543, U.S. Publication No. 20020081721, PCT Publication Nos. WO00/47757, WO00/24916, and WO96/17947). AAV vectors are typically produced in these cell types by one plasmid containing the ITR-flanked expression cassette, and one or more additional plasmids providing the additional AAV and helper virus genes.

AAV of any serotype may be used in the present disclosure. Similarly, it is contemplated that any AV type may be used, and a person of skill in the art will be able to identify AAV and AV types suitable for the production of their desired recombinant AAV vector (rAAV). AAV and AV particles may be purified, for example, by affinity chromatography, iodixanol gradient, or CsCl gradient.

The genome of wild-type AAV is single-stranded DNA and is 4.7 kb. AAV vectors may have single-stranded genomes that are 4.7 kb in size, or are larger or smaller than 4.7 kb, including oversized genomes that are as large as 5.2 kb, or as small as 3.0 kb. Further, vector genomes may be substantially self-complementary, so that within the virus the genome is substantially double stranded. AAV vectors containing genomes of all types are suitable for use in the method of the instant disclosure.

As discussed above, AAV requires co-infection with a helper virus in order to enter the infectious phase of its life cycle. Helper viruses include Adenovirus (AV), and herpes simplex virus (HSV), and systems exist for producing AAV in insect cells using baculovirus. It has also been proposed that papilloma viruses may also provide a helper function for AAV (see, e.g., Hermonat et al., Molecular Therapy 9, S289-S290 (2004)). Helper viruses include any virus capable of creating and allowing AAV replication. AV is a nonenveloped nuclear DNA virus with a double-stranded DNA genome of approximately 36 kb. AV is capable of rescuing latent AAV provirus in a cell, by providing Ela, Elb55K, E2a, E4orf6, and VA genes, and allowing AAV replication and encapsidation. HSV is a family of viruses that have a relatively large double-stranded linear DNA genome encapsidated in an icosahedral capsid, which is wrapped in a lipid bilayer envelope. HSV are infectious and highly transmissible. The following HSV-1 replication proteins were identified as necessary for AAV replication: the helicase/primase complex (UL5, UL8, and UL52) and the DNA binding protein ICP8 encoded by the UL29 gene, with other proteins enhancing the helper function. An AAV packaging system serves two purposes: it circumvents the problem of the transfection process, and provide a production technology based on the use of one or several helper functions.

Production of rAAV

General principles of rAAV can be reviewed elsewhere (See, e.g., Carter, 1992, Current Opinions in Biotechnology, 3:533-539; and Muzyczka, 1992, Curr. Topics in Microbiol. and Immunol., 158:97-129). In general terms, to allow for production of rAAV, the cell must be provided with AAV ITRs, which may, for example, flank a heterologous nucleotide sequence of interest, AAV rep and cap gene functions, as well as additional helper functions. These may be provided to the cell using any number of appropriate plasmids or vectors. Additional helper functions can be provided by, for example, an adenovirus (AV) infection, by a plasmid that carries all of the required AV helper function genes, or by other viruses such as HSV or baculovirus. Any genes, gene functions, or genetic material necessary for rAAV production by the cell may transiently exist within the cell, or be stably inserted into the cell genome. rAAV production methods suitable for use with the methods of the current disclosure include those disclosed in Clark et al., Human Gene Therapy 6:1329-1341 (1995), Martin et al., Human Gene Therapy Methods 24:253-269 (2013), Thorne et al., Human Gene Therapy 20:707-714 (2009), Fraser Wright, Human Gene Therapy 20:698-706 (2009), and Virag et al., Human Gene Therapy 20:807-817 (2009). The two main approaches for AAV production systems are recombinant adeno-associated virus (rAAV) packaging cell line and adeno-associated virus (rAAV) producer cell line.

Recombinant Adeno-Associated Virus (rAAV) Packaging and/or Producer Cell Line

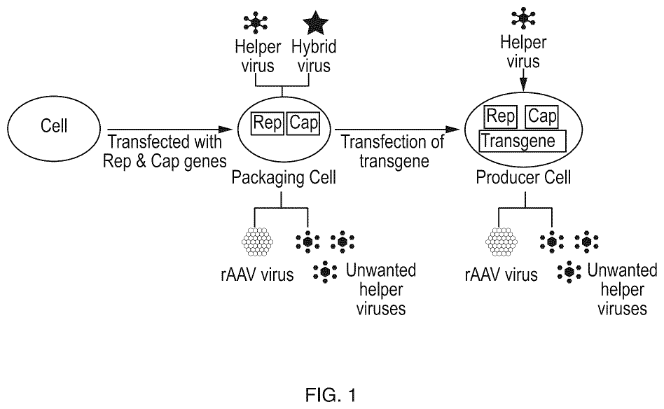

A rAAV packaging cell line can be produced by allowing cellular expression of AAV genetic elements described herein. The stable transfection of a cell line (e.g., HEK293, HeLa) with a plasmid encoding the AAV rep and cap genes can result in production of a packaging cell line. This rAAV packaging cell line can be co-infected with two different adenoviruses (helper virus and hybrid virus that contains the AAV gene-therapy elements) to produce rAAV particles. Alternatively, the stable transfection of the packaging cells with a plasmid containing the rAAV vector or their infection with a rAAV vector leads to a rAAV producer cell line. The infection of the producer cells with a helper virus leads to production of rAAV. FIG. 1 illustrates the packaging and producer cell lines.

In certain embodiments of the present disclosure, the rAAV packaging cell line comprising AAV rep and cap gene functions is engineered to increase the rAAV titer.

In one aspect, the present disclosure provides a rAAV packaging cell line comprising cells in which expression of one or more genes and/or proteins is reduced compared to control parental cells. For example, expression of ATP5EP2, LINC00319, CYP3A7, ABCA10, NOG, RGMA, SPANXN3, PGA5, MYRIP, KCNN2, and/or NALCN-AS1 is reduced compared to control parental cells.

In some embodiments, the present disclosure provides a rAAV packaging cell line comprising cells in which expression of KCNN2, LINC00319, RGMA, and SPANXN3 is reduced compared to control parental cells.

In other embodiments, the present disclosure provides a rAAV producer cell line comprising cells in which expression of one or more genes and/or proteins is reduced compared to control parental cells. For example, expression of ATP5EP2, LINC00319, CYP3A7, ABCA10, NOG, RGMA, SPANXN3, PGA5, MYRIP, KCNN2, and/or NALCN-AS1 is reduced compared to control parental cells. In some embodiments, the rAAV producer cell line of the present disclosure has been engineered to reduce gene expression of KCNN2, LINC00319, RGMA, and SPANXN3.

In certain embodiments, the cell line of the present disclosure may be in an adherent or suspension form.

In certain embodiments, the cell line of the present disclosure (e.g., rAAV packaging and/or producer cell line) is a mammalian cell line (e.g., HeLa, human embryonic kidney (HEK) 293, COS, A549, or Vero cell line). In certain embodiments, the cell line is an insect cell line (e.g., Sf9, Sf-21, Tn-368, or BTI-Tn-5B1-4).

Method of Generating a rAAV Producer Cell Line

In some embodiments, the present disclosure provides a method of generating a producer cell line by delivering a rAAV vector to an engineered rAAV packaging cell line comprising cells in which the expression of ATPSEP2, LINC00319, CYP3A7, ABCA10, NOG, RGMA, SPANXN3, PGA5, MYRIP, KCNN2, and/or NALCN-AS1 is reduced compared to control cells.

In certain embodiments, the present disclosure provides a method of generating a producer cell line by delivering a rAAV vector to an engineered rAAV packaging cell line comprising cells in which the expression of KCNN2, LINC00319, RGMA, and SPANXN3 is reduced compared to control parental cells.

### Supplements

As used herein, the term “supplements” refers to any compound or other material, whether chemical or biological in origin, which may be used in a media for cell culture to increase rAAV titers or to assay for increases in rAAV titers. Non-limiting examples of supplements include amino acids, salts, metals, sugars, lipids, nucleic acids, hormones, vitamins, fatty acids, proteins, enzymes, nucleosides, metabolites, surfactants, emulsifiers, inorganic salts, and polymers. In certain embodiments, the one or more supplements added to the rAAV packaging and/or producer cell line of the present disclosure is a glucocorticoid analog. In certain embodiments, the one or more supplements added to the rAAV packaging and/or producer cell line includes dexamethasone, hydrocortisone, prednisolone, methylprednisolone, betamethasone, cortisone, prednisone, budesonide, and/or triamcinolone.

In certain embodiments, the concentration of glucocorticoid analog in solution for increasing rAAV titer can be greater than or equal to 1 μM, greater than or equal to 0.1 μM, greater than or equal to 0.01 μM, between 0 and 1 μM, between 0 and 0.1 μM, between 0 and 0.01 μM, between 0.01 and 1 μM, or between 0.01 and 0.1 μM.

As used herein, “supplemented cell line” refers to a cell line (e.g., rAAV packaging and/or producer cell line) in which one or more supplements (e.g., glucocorticoid analogs) have been added to increase rAAV titer. As used herein, “non-supplemented cell line” refers to a cell line (e.g., rAAV packaging and/or producer cell line) not exposed to a supplement or supplements for increasing rAAV titer. As used herein, the terms “non-supplemented” and “unsupplemented” are used interchangeably to refer to culture conditions where the cell line (e.g., rAAV packaging and/or producer cell line) is not exposed to a supplement or supplements for increasing rAAV titer.

Method of Identifying One or More Genes Relevant to rAAV Production

The present disclosure is, in part, directed to a method of identifying one or more genes that are relevant to the production of rAAV by comparing global gene expression patterns in supplemented and non-supplemented cell lines.

The term “global gene expression” is well known in the art (See Wang Z. et al, Nature Reviews Genetics, 10 (1), 57-63 (2009)). The term “global gene expression” refers to one or more sets of data that contain information regarding different aspects of gene expression. The data set optionally includes information regarding: the presence of target-transcripts in cell or cell-derived samples; the relative and absolute abundance levels of target transcripts; the ability of various treatments (e.g., addition of supplements) to modulate expression of specific genes; and the ability of various treatments (e.g., addition of supplements) to change expression of specific genes to different levels.

The term “differentially expressed” is well known in the art (see Wang Z. et al, Nature Reviews Genetics, 10 (1), 57-63 (2009), Ozsolak, F. et al Nature Reviews Genetics, 12 (2), 87-98 (2011), Han, Y. et al Bioinformatics and Biology Insights, 9, 29-46 (2015)).

In certain embodiments, the cell line (e.g., rAAV packaging and/or producer cell line) of the present disclosure is supplemented with one or more supplements that increase the production of rAAV. In some embodiments, RNA samples are extracted from one or more cell lines (supplemented and non-supplemented) using any of well-known procedures. For example, total RNA can be purified from cells using silica-based isolation in an automation-compatible, 96-well format, such as the Rneasy® purification platform (Qiagen, Inc.; Valencia, Calif.).

Patterns of gene expression in expressed RNA samples can be evaluated by either (or both) qualitative and quantitative measures. In some embodiments, it is useful to quantitate the level of expression of a gene relative to other expression products, and/or relative to a control sequence. One convenient and broadly applicable method of determining relative expression is to compare the expression of one or more genes of interest to the expression of a control gene, such as a housekeeping gene (e.g., HPRT1, HSP70, or β-actin).

In order to ascertain whether the observed expression data, e.g., a change in gene expression profile in response to one or more treatments (e.g., addition of supplements) of a biological sample (e.g., supplemented and non-supplemented cell lines), is significant, and for example, not just a product of experimental noise or population heterogeneity, an estimate of a probability distribution can be constructed for each genetic and phenotypic endpoint in each biological sample. Construction of the estimated population distribution involves running multiple independent experiments for each treatment, e.g., all experiments are run in duplicate, triplicate, quadruplicate or the like. The expression data from multiple biological samples (e.g., supplemented and non-supplemented cell lines) can be grouped, or clustered, using multivariate statistics. Analysis of the data can produce a list of genes that are differentially expressed in response to treatment, for example, between supplemented and non-supplemented cell lines. The list of differentially expressed genes can be filtered using various gene filtering methodologies to identify one or more genes that are useful for increasing production of rAAV.

In some embodiments, the present disclosure is directed to methods of identifying one or more genes from a list of genes differentially expressed between supplemented and non-supplemented cell lines that are relevant to the production of rAAV. In certain embodiments, the cell line is a eukaryotic cell line. In certain embodiments, the cell line is a human cell line. In certain embodiments, the cell line is a HeLa cell line or a HEK293 cell line. In certain embodiments, global gene expression is measured across different cell lines (e.g., between a non-supplemented HeLa and a supplemented HeLa cell line, between a non-supplemented HEK 293 and a supplemented HEK 293 cell line, between a non-supplemented HeLa and a supplemented HEK 293 cell line, between a non-supplemented HeLa and a non-supplemented HEK 293 cell line, between a supplemented HeLa and a supplemented HEK 293 cell line) to identify one or more genes that are relevant to the production of rAAV. In certain embodiments, the global gene expression data from a supplemented HEK 293 and a supplemented HeLa can be combined and compared to the combined global gene expression data from a non-supplemented HEK 293 and a non-supplemented HeLa cell line to identify one or more genes that are relevant to the production of rAAV.

In certain embodiments, the present disclosure provides a method of producing a rAAV packaging and/or producer cell line to promote increased production of rAAV. In some embodiments, rAAV production is increased by modulating the expression of one or more genes and/or proteins identified from a list of genes that are differentially expressed between supplemented and non-supplemented rAAV producer cell lines. In certain embodiments, the titer of rAAV is increased by modulating the expression of one or more genes and/or proteins identified from a list of differentially expressed genes between supplemented and non-supplemented rAAV producer cell lines. In some embodiments, the rAAV titer is increased at least 1.5 fold (e.g., 2 fold, 3 fold, 4 fold, 5 fold, 6 fold, 7 fold, 8 fold, 9 fold, 10 fold, 20 fold, or 30 fold) compared to the rAAV titer produced by a cell line without the modulation of expression of the corresponding gene(s) and/or protein(s).

Modulated Genes and/or Proteins

In certain embodiments, the present disclosure provides a list of genes that when modulated (individually or in combinations) in a rAAV packaging and/or producer cell line enhance the production of rAAV.

ATP synthase F1 subunit epsilon pseudogene 2 (also known as ATP5EP2) encodes the ATP synthase subunit epsilon-like protein, mitochondrial. ATP5EP2 is a mitochondrial membrane ATP synthase that produces ATP from ADP in the presence of a proton gradient across the membrane which is generated by electron transport complexes of the respiratory chain. Examples of human ATP5EP2 sequences are available under the reference sequence NM_006886.4 (SEQ ID NO: 43) or NG_053163.1 (SEQ ID NO: 44) in the NCBI nucleotide database (nucleotide sequence).

Long Intergenic Non-Protein Coding RNA 319 (also known as LINC00319) is an RNA gene, and is affiliated with the non-coding RNA class. Long non-coding RNAs (lncRNAs) have been shown to play important regulatory roles in the pathogenesis and progression of multiple cancers. Examples of LINC00319 sequences are available under the reference sequence NM_194309 (SEQ ID NO: 45) or NR_026960.1 (SEQ ID NO: 46) in the NCBI nucleotide database (nucleotide sequence).

Cytochrome P450 Family 3 Subfamily A Member 7 (also known as CYP3A7) is a gene that encodes a member of the cytochrome P450 superfamily of enzymes, which participate in drug metabolism and the synthesis of cholesterol, steroids and other lipids. This enzyme hydroxylates testosterone and dehydroepiandrosterone 3-sulphate, which is involved in the formation of estriol during pregnancy. This gene is part of a cluster of related genes on chromosome 7q21.1. Examples of CYP3A7 sequences are available under the reference sequence NM_000765 (SEQ ID NO: 47) in the NCBI nucleotide database (nucleotide sequence).

ATP Binding Cassette Subfamily A Member 10 (also known as ABCA10) encodes a membrane-associated protein that belongs to a member of the superfamily of ATP-binding cassette (ABC) transporters. ABC proteins transport various molecules across extra- and intracellular membranes. ABC genes are divided into seven distinct subfamilies (ABC1, MDR/TAP, MRP, ALD, OABP, GCN20, and White). ABCA10 is a member of the ABC1 subfamily. Members of the ABC1 subfamily comprise the only major ABC subfamily found exclusively in multicellular eukaryotes. This gene is clustered among four other ABC1 family members on 17q24. Examples of ABCA10 sequences are available under the reference sequence NM_080282.3 (SEQ ID NO: 48) in the NCBI nucleotide database (nucleotide sequence).

Noggin (also known as NOG) encodes a secreted polypeptide that binds and inactivates members of the transforming growth factor-beta (TGF-beta) superfamily signaling proteins, such as bone morphogenetic protein-4 (BMP4). Without being bound by theory, it is believed that by diffusing through extracellular matrices more efficiently than members of the TGF-beta superfamily, this protein may have a principal role in creating morphogenic gradients. NOG appears to have pleiotropic effect, both early in development as well as in later stages. Examples of NOG sequences are available under the reference sequence NM_005450.4 (SEQ ID NO: 49) in the NCBI nucleotide database (nucleotide sequence).

Repulsive Guidance Molecule BMP Co-Receptor A (also known as RGMA) is a gene that encodes a member of the repulsive guidance molecule family. The encoded protein is a glycosylphosphatidylinositol-anchored glycoprotein that functions as an axon guidance protein in the developing and adult central nervous system. This protein may also function as a tumor suppressor in some cancers. Examples of RGMA sequences are available under the reference sequence NM_020211.2 (SEQ ID NO: 50) or NM_001166283.1 (SEQ ID NO: 51) in the NCBI nucleotide database (nucleotide sequence).

SPANX (Sperm protein associated with the nucleus on the X chromosome) Family Member N3 (also known as SPANXN3) is a protein coding gene. Examples of SPANXN3 sequences are available under the reference sequence NM_001009609 (SEQ ID NO: 52) in the NCBI nucleotide database (nucleotide sequence).

Pepsinogen-5, Group I (also known as PGA5 or Pepsinogen A) encodes a protein precursor of the digestive enzyme pepsin, a member of the peptidase A1 family of endopeptidases. The encoded precursor is secreted by gastric chief cells and undergoes autocatalytic cleavage in acidic conditions to form the active enzyme, which functions in the digestion of dietary proteins. This gene is found in a cluster of related genes on chromosome 11, each of which encodes one of multiple pepsinogens. Examples of PGA5 sequences are available under the reference sequence NM_014224.4 (SEQ ID NO: 53) in the NCBI nucleotide database (nucleotide sequence).

Myosin VIIA And Rab Interacting Protein (also known as MYRIP) encodes a Rab effector protein involved in melanosome transport which serves as link between melanosome-bound RAB27A and the motor proteins MYO5A and MYO7A. This Rab effector protein functions as a protein kinase A-anchoring protein (AKAP) and may act as a scaffolding protein that links PKA to components of the exocytosis machinery, thus facilitating exocytosis, including insulin release. Examples of MYRIP sequences are available under the reference sequence NM_015460 (SEQ ID NO: 54) or NM_001284423.1 (SEQ ID NO: 55) in the NCBI nucleotide database (nucleotide sequence).

Potassium Calcium-Activated Channel Subfamily N Member 2 (also known as KCNN2) gene is a member of the KCNN family of potassium channel genes. The encoded protein is an integral membrane protein that forms a voltage-independent calcium-activated channel with three other calmodulin-binding subunits. Alternate splicing of this gene results in multiple transcript variants. Examples of KCNN2 sequences are available under the reference sequence NM_170775.2 (SEQ ID NO: 56) or NM_001278204.1 (SEQ ID NO: 57) in the NCBI nucleotide database (nucleotide sequence).

NALCN Antisense RNA 1 (also known as NALCN-AS1) is an RNA gene, and is affiliated with the non-coding RNA class. Examples of NALCN-AS1 sequences are available under the reference sequence NW_011332700.1 (SEQ ID NO: 58) or NR_047687.1 (SEQ ID NO: 59) in the NCBI nucleotide database (nucleotide sequence).

In certain embodiments, the present disclosure provides a rAAV packaging and/or producer cell line comprising cells in which the expression of ATP5EP2, LINC00319, CYP3A7, ABCA10, NOG, RGMA, SPANXN3, PGA5, MYRIP, KCNN2, and/or NALCN-AS1 is reduced compared to control parental cells.

In certain embodiments, the present disclosure provides a rAAV packaging and/or producer cell line comprising cells in which the expression of KCNN2, LINC00319, RGMA, and SPANXN3 is reduced compared to control parental cells.

In certain embodiments, the present disclosure provides a list of genes that when modulated individually in a rAAV packaging and/or producer cell line enhance the production of rAAV compared to a control parental cell line. In some aspects, the modulation of different combination of genes in a rAAV packaging and/or producer cell line increases the production of rAAV. In some aspects, modulating the expression of at least 1, at least 2, at least 3, at least 4, at least 5, at least 6, at least 7, at least 8, at least 9, at least 10, or at least 11 genes in a rAAV packaging and/or producer cell line results in increased rAAV production compared to a control parental cell line.

Methods of Modulating One or More Genes and/or Protein

Modulating (e.g., reducing) the expression or activity of a gene (e.g., ATPSEP2, LINC00319, CYP3A7, ABCA10, NOG, RGMA, SPANXN3, PGA5, MYRIP, KCNN2, or NALCN-AS1) can be achieved by different mechanisms, including, but not limited to, altering one or more of the following: 1) gene copy number, 2) transcription or translation of a gene, 3) transcript stability or longevity, 4) the number of copies of an mRNA or miRNA, 5) the availability of a non-coding RNA or non-coding RNA target site, 6) the position or degree of post-translational modifications on a protein, or 7) the activity of a protein. Tools that can be used to modulate gene expression include but are not limited to a nuclease, a double stranded RNA (dsRNA), a small interfering RNA (siRNA), a small hairpin RNA (shRNA), a microRNA (miRNA), an antisense RNA oligonucleotide (ASO), a gene disruption, or a partial or complete gene deletion.

### Nuclease

In certain embodiments, gene modulation is achieved using zinc finger nucleases (ZFNs). Synthetic ZFNs are composed of a zinc finger binding domain fused with, e.g., a FokI DNA cleavage domain. ZFNs can be designed/engineered for editing the genome of a cell, including, but not limited to, knock out or knock in gene expression, in a wide range of organisms. Meganucleases, transcription activator-like effector nucleases (TALENs), or clustered regularly interspaced short palindromic repeats (CRISPR) associated proteins (e.g., Cas nucleases), and triplexes can also be used for genome engineering in a wide array of cell types. The described reagents can be used to target promoters, protein-encoding regions (exons), introns, 5′ and 3′ UTRs, and more.

Double Stranded RNA (dsRNA) Molecules for Modulation

In certain embodiments, double-stranded RNA (dsRNA) molecules may be used to modulate expression of one or more genes in a cell line described herein (e.g., a rAAV packaging and/or producer cell line). dsRNA molecules can be designed to antagonize one or more genes by sequence homology-based targeting of the corresponding RNA sequence. Such dsRNAs can be small interfering RNAs (siRNAs), small hairpin RNAs (shRNAs), or micro-RNAs (miRNAs). The sequence of such dsRNAs will comprise a complementary portion of the mRNA encoding the one or more genes to be modulated. This portion can be 100% complementary to the target portion within the mRNA, but lower levels of complementarity (e.g., 90% or more or 95% or more) can also be used. Typically the percent complementarity is determined over a length of contiguous nucleic acid residues. A dsRNA molecule of the disclosure may, for example, have at least 80% complementarity to the target portion within the mRNA measured over at least 10, at least 20, at least 30, at least 40, at least 50, at least 60, at least 70, at least 80, at least 90, or more nucleic acid residues. In some instances dsRNA molecule has at least 80% complementarity to the target portion of mRNA over the entire length of the dsRNA molecule.

Another gene targeting reagent that uses RNA interference (RNAi) pathways is small hairpin RNA, also referred to as shRNA. shRNAs delivered to cells via, e.g., expression constructs (e.g., plasmids, lentiviruses) have the ability to provide long term reduction of gene expression in a constitutive or regulated manner, depending upon the type of promoter employed. In one embodiment, the genome of a lentiviral particle is modified to include one or more shRNA expression cassettes that target a gene (or genes) of interest. Such lentiviruses can infect a cell, stably integrate their viral genome into the host genome, and express a shRNA in a constitutive, regulated, or (in the case where multiple shRNA are being expressed) constitutive and regulated fashion. Thus, in some embodiments shRNA can be designed to target individual variants of a single gene or multiple closely related gene family members. Individual shRNA can modulate collections of targets having similar or redundant functions or sequence motifs. The skilled person will recognize that lentiviral constructs can also incorporate cloned DNA, or ORF expression constructs.

In embodiments described herein, gene targeting reagents including small interfering RNAs (siRNA) as well as microRNAs (miRNA) can be used to modulate gene function. siRNAs and miRNAs can incorporate a wide range of chemical modifications, levels of complementarity to the target transcript of interest, and designs (see U.S. Pat. No. 8,188,060) to enhance stability, cellular delivery, specificity, and functionality. In addition, such reagents can be designed to target diverse regions of a gene (including the 5′ UTR, the open reading frame, the 3′ UTR of the mRNA), or (in some cases) the promoter/enhancer regions of the genomic DNA encoding the gene of interest. Gene modulation (e.g., reduction of gene expression, knockdown) can be achieved by introducing (into a cell) a single siRNA or miRNA or multiple siRNAs or pools of miRNAs targeting different regions of the same mRNA transcript. Synthetic siRNA/miRNA delivery can be achieved by any number of methods including but not limited to 1) self-delivery, 2) lipid-mediated delivery, 3) electroporation, or 4) vector/plasmid-based expression systems. An introduced RNA molecule may be referred to as an exogenous nucleotide sequence or polynucleotide. In some embodiments, siRNA can be designed to target individual variants of a single gene or multiple closely related gene family members.

siRNA can be used to reduce the expression of one or more genes (e.g., ATP5EP2, LINC00319, CYP3A7, ABCA10, NOG, RGMA, SPANXN3, PGA5, MYRIP, KCNN2, and/or NALCN-AS1). In some embodiments, a siRNA which comprises a nucleotide sequence selected from SEQ ID NOs: 1 to 11, or a variant thereof, is used to reduce the expression of a target gene.

TABLE 1
siRNA sequences used for reducing
expression of genes.

| | Target | |
|---|---|---|
| SEQ ID NO: | gene | siRNA Sequence* |
| SEQ ID NOS | ATP5SEP2 | Sense: GCAACAGCGUAAAAAUUGUtt |
| 1 and 32 | | (SEQ ID NO: 1) |
| | | Antisense: |
| | | ACAAUUUUUACGCUGUUGCca |
| | | (SEQ ID NO: 32) |
| SEQ ID NOS | LINC00319 | Sense: CGGUGUCCACAGUCCUUGAtt |
| 2 and 33 | | (SEQ ID NO: 2) |
| | | Antisense: |
| | | UCAAGGACUGUGGACACCGgt |
| | | (SEQ ID NO: 33) |
| SEQ ID NOS | CYP3A7 | Sense: CAAGAAAAGUUAUAAGUUUtt |
| 3 and 34 | | (SEQ ID NO: 3) |
| | | Antisense: |
| | | AAACUUAUAACUUUUCUUGga |
| | | (SEQ ID NO: 34) |
| SEQ ID NOS | NOG | Sense: CGGAGGAAGUUACAGAUGUtt |
| 4 and 35 | | (SEQ ID NO: 4) |
| | | Antisense: |
| | | ACAUCUGUAACUUCCUCCGca |
| | | (SEQ ID NO: 35) |
| SEQ ID NOS | SPANXN3 | Sense: AGAUGCAAGAGGUACCAAAtt |
| 5 and 36 | | (SEQ ID NO: 5) |
| | | Antisense: |
| | | UUUGGUACCUCUUGCAUCUca |
| | | (SEQ ID NO: 36) |
| SEQ ID NOS | MYRIP | Sense: GGUGUCGGAUGAUUUAUCAtt |
| 6 and 37 | | (SEQ ID NO: 6) |
| | | Antisense: |
| | | UGAUAAAUCAUCCGACACCtg |
| | | (SEQ ID NO: 37) |
| SEQ ID NOS | KCNN2 | Sense: GAAGCUAGAACUUACCAAAtt |
| 7 and 38 | | (SEQ ID NO: 7) |
| | | Antisense: |
| | | UUUGGUAAGUUCUAGCUUCct |
| | | (SEQ ID NO: 38) |
| SEQ ID NOS | NALCN- | Sense: GGAUGUCUUUCCUAGGAGAtt |
| 8 and 39 | AS1 | (SEQ ID NO: 8) |
| | | Antisense: |
| | | UCUCCUAGGAAAGACAUCCaa |
| | | (SEQ ID NO: 39) |
| SEQ ID NOS | RGMA | Sense: CGCUCAUCGACAAUAAUUAtt |
| 9 and 40 | | (SEQ ID NO: 9) |
| | | Antisense: |
| | | UAAUUAUUGUCGAUGAGCGgc |
| | | (SEQ ID NO: 40) |
| SEQ ID NOS | PGA5 | Sense: CACUUUAGAUGUAUCUAAUtt |
| 10 and 41 | | (SEQ ID NO: 10) |
| | | Antisense: |
| | | AUUAGAUACAUCUAAAGUGgg |
| | | (SEQ ID NO: 41) |
| SEQ ID NOS | ABCA10 | Sense: GGAGCAUAAAGUAGACCGAtt |
| 11 and 42 | | (SEQ ID NO: 11) |
| | | Antisense: |
| | | UCGGUCUACUUUAUGCUCCtt |
| | | (SEQ ID NO: 42) |

*siRNA sequences (sense and antisense) used for reducing expression of genes. Lower case nucleotides in the sequences represent 3′ overhang.

In some embodiments, the siRNA used to reduce the expression of ATPSEP2 comprises the nucleotide sequence of SEQ ID NO: 1, or a variant thereof. For example, in some embodiments, the siRNA comprises the nucleotide sequence of SEQ ID NO: 1 in the sense strand and the nucleotide sequence of SEQ ID NO: 32 in the anti-sense strand.

In some embodiments, the siRNA used to reduce the expression of LINC00319 comprises the nucleotide sequence of SEQ ID NO: 2, or a variant thereof. For example, in some embodiments, the siRNA comprises the nucleotide sequence of SEQ ID NO: 2 in the sense strand and the nucleotide sequence of SEQ ID NO: 33 in the anti-sense strand.

In some embodiments, the siRNA used to reduce the expression of CYP3A7 comprises the nucleotide sequence of SEQ ID NO: 3, or a variant thereof. For example, in some embodiments, the siRNA comprises the nucleotide sequence of SEQ ID NO: 3 in the sense strand and the nucleotide sequence of SEQ ID NO: 34 in the anti-sense strand.

In some embodiments, the siRNA used to reduce the expression of NOG comprises the nucleotide sequence of SEQ ID NO: 4, or a variant thereof. For example, in some embodiments, the siRNA comprises the nucleotide sequence of SEQ ID NO: 4 in the sense strand and the nucleotide sequence of SEQ ID NO: 35 in the anti-sense strand.

In some embodiments, the siRNA used to reduce the expression of SPANXN3 comprises the nucleotide sequence of SEQ ID NO: 5, or a variant thereof. For example, in some embodiments, the siRNA comprises the nucleotide sequence of SEQ ID NO: 5 in the sense strand and the nucleotide sequence of SEQ ID NO: 36 in the anti-sense strand.

In some embodiments, the siRNA used to reduce the expression of MYRIP comprises the nucleotide sequence of SEQ ID NO: 6, or a variant thereof. For example, in some embodiments, the siRNA comprises the nucleotide sequence of SEQ ID NO: 6 in the sense strand and the nucleotide sequence of SEQ ID NO: 37 in the anti-sense strand.

In some embodiments, the siRNA used to reduce the expression of KCNN2 comprises the nucleotide sequence of SEQ ID NO: 7, or a variant thereof. For example, in some embodiments, the siRNA comprises the nucleotide sequence of SEQ ID NO: 7 in the sense strand and the nucleotide sequence of SEQ ID NO: 38 in the anti-sense strand.

In some embodiments, the siRNA used to reduce the expression of NALCN-AS1 comprises the nucleotide sequence of SEQ ID NO: 8, or a variant thereof. For example, in some embodiments, the siRNA comprises the nucleotide sequence of SEQ ID NO: 8 in the sense strand and the nucleotide sequence of SEQ ID NO: 39 in the anti-sense strand.

In some embodiments, the siRNA used to reduce the expression of RGMA comprises the nucleotide sequence of SEQ ID NO: 9, or a variant thereof. For example, in some embodiments, the siRNA comprises the nucleotide sequence of SEQ ID NO: 9 in the sense strand and the nucleotide sequence of SEQ ID NO: 40 in the anti-sense strand.

In some embodiments, the siRNA used to reduce the expression of PGA5 comprises the nucleotide sequence of SEQ ID NO: 10, or a variant thereof. For example, in some embodiments, the siRNA comprises the nucleotide sequence of SEQ ID NO: 10 in the sense strand and the nucleotide sequence of SEQ ID NO: 41 in the anti-sense strand.

In some embodiments, the siRNA used to reduce the expression of ABCA10 comprises the nucleotide sequence of SEQ ID NO: 11, or a variant thereof. For example, in some embodiments, the siRNA comprises the nucleotide sequence of SEQ ID NO: 11 in the sense strand and the nucleotide sequence of SEQ ID NO: 42 in the anti-sense strand.

### Antisense RNA Oligonucleotide (ASO)

Antisense RNA oligonucleotide (ASO), can be used to modulate expression of one or more genes in a rAAV packaging and/or producer cell line. Typically, ASOs are used to reduce expression of one or more genes. Using known techniques and based on a knowledge of the sequence of the one or more gene to be modulated, ASO molecules can be designed to antagonize the one or more genes by sequence homology-based targeting of the corresponding RNA. The ASO sequence can comprise nucleotide sequence that is complementary to a target portion of the mRNA or lncRNA produced from the one or more genes. This portion can be 100% complementary to the target portion within the mRNA or lncRNA but lower levels of complementarity (e.g., 90% or more or 95% or more) can also be used.

In some embodiments, the ASO can be an antisense RNA oligonucleotide wherein at least one nucleoside linkage of the sequence is a phosphorothioate linkage, a phosphorodithioate linkage, a phosphotriester linkage, an alkylphosphonate linkage, an aminoalkylphosphotriester linkage, an alkylene phosphonate linkage, a phosphinate linkage, a phosphoramidate linkage, and an aminoalkylphosphoramidate linkage, a thiophosphoramidate linkage, thionoalkylphosphonate linkage, a thionoalkylphosphotriester linkage, a thiophosphate linkage, a selenophosphate linkage, or a boranophosphate linkage. In a particular embodiment, at least one internucleoside linkage of the antisense RNA oligonucleotide sequence is a phosphorothioate linkage. In some embodiments, all of the internucleoside linkages of the antisense RNA oligonucleotide sequence are phosphorothioate linkages.

### CRISPR Genome Editing

In some embodiments, modulation of gene expression in a rAAV packaging and/or producer cell line is carried out using CRISPR genome editing. The CRISPR genome editing typically comprises two distinct components: (1) a guide RNA and (2) an endonuclease, specifically a CRISPR associated (Cas) nuclease (e.g., Cas9). The guide RNA is a combination of the endogenous bacterial crRNA and tracrRNA into a single chimeric guide RNA (gRNA) transcript. Without being bound by theory, it is believed that when gRNA and the Cas are expressed in the cell, the genomic target sequence can be modified or permanently disrupted.

The gRNA/Cas complex is recruited to the target sequence by base-pairing between the gRNA sequence and the complement to the target DNA sequence in the gene for reduction (e.g., ATP5EP2, LINC00319, CYP3A7, ABCA10, NOG, RGMA, SPANXN3, PGA5, MYRIP, KCNN2, or NALCN-AS1). For successful binding of Cas, the genomic target sequence must also contain the correct Protospacer Adjacent Motif (PAM) sequence immediately following the target sequence. The binding of the gRNA/Cas complex localizes the Cas to the genomic target sequence in the one or more genes of the present disclosure so that the wild-type Cas can cut both strands of DNA causing a double strand break. This can be repaired through one of two general repair pathways: (1) the non-homologous end joining DNA repair pathway or (2) the homology directed repair pathway. The non-homologous repair pathway can result in inserts/deletions at the double strand break that can lead to frameshifts and/or premature stop codons, effectively disrupting the open reading frame of the target gene. The homology directed repair pathway requires the presence of a repair template, which is used to fix the double strand break.

Any appropriate gRNA pair may be used for CRISPR genome editing. Typically gRNA pairs are used to reduce expression of one or more genes (e.g., ATP5EP2, LINC00319, CYP3A7, ABCA10, NOG, RGMA, SPANXN3, PGA5, MYRIP, KCNN2, and NALCN-AS1). In some embodiments described herein, a gRNA pair is used to modulate (e.g., reduce or eliminate/knockout) expression of ATP5EP2, LINC00319, CYP3A7, ABCA10, NOG, RGMA, SPANXN3, PGA5, MYRIP, KCNN2, and/or NALCN-AS1.

gRNA pairs can be designed using known techniques and based on a knowledge of the sequence of the one or more genes to be modulated, typically using any publicly available appropriate computer program. Knock out packaging and/or producer cells may be generated using any appropriate technique, with standard techniques being known in the art and suitable kits being commercially available.

gRNA pairs can be delivered to a producer cell line of the disclosure by any appropriate means. Suitable techniques are known in the art and include the use of plasmid, viral and bacterial vectors to deliver the gRNA pairs to the producer cell line. Typically, a gRNA pair is delivered using plasmid DNA.

gRNA pairs may be used to reduce the expression of one or more of genes (e.g., ATP5EP2, LINC00319, CYP3A7, ABCA10, NOG, RGMA, SPANXN3, PGA5, MYRIP, KCNN2, and NALCN-AS1). Multiple gRNA pairs may be used to modulate the expression of a gene. In some embodiments described herein, gRNA pairs are used to reduce the expression of at least one of ATPSEP2, LINC00319, CYP3A7, ABCA10, NOG, RGMA, SPANXN3, PGA5, MYRIP, KCNN2, or NALCN-AS1. Multiple gRNA pairs may be used to modulate the expression of KCNN2, LINC00319, RGMA, and SPANXN3. In some embodiments, gRNAs may be modified to enhance editing efficiency by increasing binding to the target site and inhibiting nuclease degradation. In certain embodiments, these modifications may be 2′ O-methyl analogs and 3′ phosphorothioate internucleotide linkages in the terminal three nucleotides on both 5′ and 3′ ends of the gRNA. Exemplary target DNA sequences targeted by gRNA pairs used to modulate gene expression of one or more genes may comprise any one of nucleotide sequences selected from SEQ ID NOs: 16-31 listed in Table 2, or variants thereof.

TABLE 2
Exemplary target region sequences of
gRNA pairs (SEQ ID NO: 12-15) and
target DNA sequences (SEQ ID NOs: 16-31)

| | |
|---|---|
| SEQ ID NO: | Sequence |

KCNN2

| | |
|---|---|
| SEQ ID NO: 12 | UUGCCACUACAGCUACCACC |
| SEQ ID NO: 13 | CCAAUGUACUCAGGGAAACA |
| SEQ ID NO: 14 | AGUCCACCAAAGUGUUUGCU |
| SEQ ID NO: 15 | AAAGGAGUCUGCUUACUUAC |

KCNN2

| | |
|---|---|
| SEQ ID NO: 16 | TTGCCACTACAGCTACCACC |
| SEQ ID NO: 17 | CCAATGTACTCAGGGAAACA |
| SEQ ID NO: 18 | AGTCCACCAAAGTGTTTGCT |
| SEQ ID NO: 19 | AAAGGAGTCTGCTTACTTAC |

RGMA

| | |
|---|---|
| SEQ ID NO: 20 | CTTCTCGTAATGGCAGATCT |
| SEQ ID NO: 21 | GCACTTGAGGATCTTGCACG |
| SEQ ID NO: 22 | GAGGTCCTCTATGCCATGGA |
| SEQ ID NO: 23 | CCATACCCATCCATCCAGCT |

SPANXN3

| | |
|---|---|
| SEQ ID NO: 24 | CCCATGTGAAGGACCTTCAA |
| SEQ ID NO: 25 | GTTCTTCAAACTCTGTTCGG |
| SEQ ID NO: 26 | GAAGGCGTAGACTTATCTGA |
| SEQ ID NO: 27 | AGCCAACTTCCAGCACCAAT |

LINC00319

| | |
|---|---|
| SEQ ID NO: 28 | GGGCAATGGACCTTCTGCCT |
| SEQ ID NO: 29 | GGCTGCGGGGCAGAGGGCAA |
| SEQ ID NO: 30 | CGGGCAGGCTGCGGGGCAGA |
| SEQ ID NO: 31 | ACGGGCAGGCTGCGGGGCAG |

For example, gRNA pairs used to target KCNN2 can comprise a sequence selected from the nucleotide sequences of SEQ ID NO: 12-15 (shown in Table 2). In some embodiments, a gRNA pair used to target KCNN2 comprises a first gRNA molecule comprising the sequence of SEQ ID NO: 12 and a second gRNA molecule comprising the sequence of SEQ ID NO: 13. In some embodiments, a gRNA pair used to target KCNN2 comprises a first gRNA molecule comprising or having the sequence of SEQ ID NO: 14 and a second gRNA molecule comprising or having the sequence of SEQ ID NO: 15.

In some embodiments, a gRNA molecule to target KCNN2 is a 2′ O-methyl analog comprising 3′ phosphorothioate internucleotide linkages in the terminal three nucleotides on either or both its 5′ and 3′ ends and comprises the sequence of SEQ ID NO: 12, 13, 14, or 15.

A variant gRNA sequence may have at least 80% sequence identity to a sequence of the present disclosure, measured over any appropriate length of sequence. Typically the percent sequence identity is determined over a length of contiguous nucleic acids. A variant gRNA sequence of the present disclosure can, for example, have at least 80% sequence identity to a sequence of the present disclosure measured over at least 10, at least 11, at least 12, at least 13, at least 14, at least 15, at least 16, at least 17, at least 18, at least 19, at least 20, at least 21, at least 22, at least 23, at least 24, or more nucleic acid residues. In some embodiments, the variant gRNA molecule has at least 80% sequence identity with the gRNA molecule of the present disclosure over the entire length of the variant gRNA molecule. In some embodiments, a variant gRNA molecule of the present disclosure can be a variant of one or more of the gRNA molecules whose target regions are complementary to a target sequence of one of SEQ ID NOs: 16 to 30. gRNA pairs of the present disclosure may comprise a variant of one or both of two gRNA sequences in the pair targeting a gene, e.g., a gene selected from ATP5EP2, LINC00319, CYP3A7, ABCA10, NOG, RGMA, SPANXN3, PGA5, MYRIP, KCNN2, and NALCN-AS1. For example, a variant of the gRNA pair comprising a first gRNA molecule comprising the sequence of SEQ ID NO: 12 and a second gRNA molecule comprising the sequence of SEQ ID NO: 13 may comprise 1) a first gRNA molecule comprising a variant of the sequence of SEQ ID NO: 12, 2) a second gRNA molecule comprising a variant of the sequence of SEQ ID NO: 13, or 3) both.

### Modulation at Protein Level

In another embodiment, modulation of expression and/or activity of a gene takes place at the protein (e.g., polypeptide) level. By way of example, reduction of gene function at the protein level can be achieved by methods including, but not limited to, targeting the protein with a small molecule, a peptide, an aptamer, destabilizing domains, or other methods that can e.g., down-regulate the activity or enhance the rate of degradation of a gene product. Alternatively, the expressed protein may be modified to reduce or eliminate biological activity through site-directed mutagenesis and/or the incorporation of missense or nonsense mutations. In some embodiments, a small molecule that binds, e.g., an active site and inhibits the function of a target protein can be added to, e.g., the cell culture media and thereby be introduced into a packaging and/or producer cell. Alternatively, target protein function can be modulated by introducing, e.g., a peptide into a cell (e.g., a packaging and/or producer cell) that for instance prevents protein-protein interactions (see Shangary et. al., (2009) Annual Review of Pharmacology and Toxicology 49:223). Such peptides can be introduced into a cell (e.g., a packaging and/or producer cell) by, for example, transfection or electroporation, or via an expression construct. Alternatively, peptides can be introduced into a cell (e.g., a packaging and/or producer cell) by adding (e.g., through conjugation) one or more moieties that facilitate cellular delivery, or supercharging molecules to enhance self-delivery. Techniques for expressing a peptide include, but are not limited to, fusion of the peptide to a scaffold, or attachment of a signal sequence, to stabilize or direct the peptide to a position or compartment of interest, respectively. In certain embodiments, a rAAV packaging and/or producer cell line comprises cells which have been engineered to reduce the expression and/or activity of a gene product expressed from ATP5EP2, LINC00319, CYP3A7, ABCA10, NOG, RGMA, SPANXN3, PGA5, MYRIP, KCNN2, and/or NALCN-AS1 using any of the aforementioned methods.

Effect of Modulation on Expression of One or More Genes and/or Proteins

In certain embodiments, methods of modulations described in the present disclosure can be utilized to generate a rAAV packaging and/or producer cell line that produces high titers of rAAV. In certain embodiments, methods of modulations described in the present disclosure can result in a significant reduction in expression of one or more genes (e.g., ATP5EP2, LINC00319, CYP3A7, ABCA10, NOG, RGMA, SPANXN3, PGA5, MYRIP, KCNN2, and/or NALCN-AS1) and/or a significant reduction in the activity of a protein expressed by one or more genes (e.g., a reduction of at least 5%, at least 10%, at least 20%, or greater reduction). In certain embodiments, expression of a target gene is reduced from about 40% to about 100% (for example, from about 40% to about 95%, from about 40% to about 90%, from about 40% to about 85%, from about 40% to about 80%, from about 40% to about 75%, from about 40% to about 70%, from about 40% to about 65%, from about 40% to about 60%, from about 40% to about 55%, from about 40% to about 50%, from about 40% to about 45%, from about 45% to about 100%, from about 50% to about 100%, from about 55% to about 100%, from about 60% to about 100%, from about 65% to about 100%, from about 70% to about 100%, from about 75% to about 100%, from about 80% to about 100%, from about 85% to about 100%, from about 90% to about 100%, from about 95% to about 100%; or about 40%, about 50%, about 60%, about 70%, about 80%, about 90%, about 100%).

In certain embodiments, methods of modulation described in the present disclosure can result in a significant reduction in activity of a protein or RNA expressed by a target gene (e.g., ATP5EP2, LINC00319, CYP3A7, ABCA10, NOG, RGMA, SPANXN3, PGA5, MYRIP, KCNN2, and/or NALCN-AS1). For example, methods described herein can result in at least 5%, at least 10%, at least 20% or greater reduction in activity of a protein or RNA expressed by a target gene. In certain embodiments, target gene protein or RNA activity is reduced from about 40% to about 100% (for example, from about 40% to about 95%, from about 40% to about 90%, from about 40% to about 85%, from about 40% to about 80%, from about 40% to about 75%, from about 40% to about 70%, from about 40% to about 65%, from about 40% to about 60%, from about 40% to about 55%, from about 40% to about 50%, from about 40% to about 45%, from about 45% to about 100%, from about 50% to about 100%, from about 55% to about 100%, from about 60% to about 100%, from about 65% to about 100%, from about 70% to about 100%, from about 75% to about 100%, from about 80% to about 100%, from about 85% to about 100%, from about 90% to about 100%, from about 95% to about 100%; or about 40%, about 50%, about 60%, about 70%, about 80%, about 90%, about 100%). Furthermore, modulation of one or more genes can result in modulation of multiple genes (e.g., by miRNAs).

In certain embodiments, methods of modulation described in the present disclosure can result in a significant reduction in expression of gene product (e.g., a gene product of ATP5EP2, LINC00319, CYP3A7, ABCA10, NOG, RGMA, SPANXN3, PGA5, MYRIP, KCNN2, and/or NALCN-AS1) (e.g., at least 5%, at least 10%, at least 20% or greater reduction). In certain embodiments, expression of a gene product is reduced from about 40% to about 100% (for example, from about 40% to about 95%, from about 40% to about 90%, from about 40% to about 85%, from about 40% to about 80%, from about 40% to about 75%, from about 40% to about 70%, from about 40% to about 65%, from about 40% to about 60%, from about 40% to about 55%, from about 40% to about 50%, from about 40% to about 45%, from about 45% to about 100%, from about 50% to about 100%, from about 55% to about 100%, from about 60% to about 100%, from about 65% to about 100%, from about 70% to about 100%, from about 75% to about 100%, from about 80% to about 100%, from about 85% to about 100%, from about 90% to about 100%, from about 95% to about 100%; or about 40%, about 50%, about 60%, about 70%, about 80%, about 90%, about 100%).

In certain embodiments, methods of modulation described in the present disclosure can result in a significant reduction in expression of polypeptide or polyribonucleotide expressed from at least one of ATPSEP2, LINC00319, CYP3A7, ABCA10, NOG, RGMA, SPANXN3, PGA5, MYRIP, KCNN2, and/or NALCN-AS1 (e.g., at least 5%, at least 10%, at least 20% or greater reduction). In certain embodiments, expression of polypeptide or polyribonucleotide is reduced from about 40% to about 100% (for example, from about 40% to about 95%, from about 40% to about 90%, from about 40% to about 85%, from about 40% to about 80%, from about 40% to about 75%, from about 40% to about 70%, from about 40% to about 65%, from about 40% to about 60%, from about 40% to about 55%, from about 40% to about 50%, from about 40% to about 45%, from about 45% to about 100%, from about 50% to about 100%, from about 55% to about 100%, from about 60% to about 100%, from about 65% to about 100%, from about 70% to about 100%, from about 75% to about 100%, from about 80% to about 100%, from about 85% to about 100%, from about 90% to about 100%, from about 95% to about 100%; or about 40%, about 50%, about 60%, about 70%, about 80%, about 90%, about 100%).

In certain embodiments, methods of modulation described in the present disclosure can result in a significant reduction in activity of a polypeptide or polyribonucleotide expressed from at least one of ATPSEP2, LINC00319, CYP3A7, ABCA10, NOG, RGMA, SPANXN3, PGA5, MYRIP, KCNN2, and/or NALCN-AS1 (e.g., at least 5%, at least 10%, at least 20% or greater reduction). In certain embodiments, activity of expressed polypeptide or polyribonucleotide is reduced from about 40% to about 100% (for example, from about 40% to about 95%, from about 40% to about 90%, from about 40% to about 85%, from about 40% to about 80%, from about 40% to about 75%, from about 40% to about 70%, from about 40% to about 65%, from about 40% to about 60%, from about 40% to about 55%, from about 40% to about 50%, from about 40% to about 45%, from about 45% to about 100%, from about 50% to about 100%, from about 55% to about 100%, from about 60% to about 100%, from about 65% to about 100%, from about 70% to about 100%, from about 75% to about 100%, from about 80% to about 100%, from about 85% to about 100%, from about 90% to about 100%, from about 95% to about 100%; or about 40%, about 50%, about 60%, about 70%, about 80%, about 90%, about 100%).

In certain embodiments, reduction in expression and/or activity of one or more genes, proteins, or RNAs in a rAAV packaging and/or producer cell line is maintained for about 5 days (e.g., about 6 hours, about 12 hours, about 1 day, about 2 days, about 3 days, about 4 days, about 5 days, about 6 days, about 7 days, about 8 days, about 9 days, about 10 days or more).

In certain embodiments, reduction in expression and/or activity of one or more genes, proteins, or RNAs in a rAAV packaging and/or producer cell line is intended to be maintained indefinitely or permanently, e.g., through the use of a gene disruption or a partial or complete gene deletion.

In certain embodiments, reduction in expression and/or activity of one or more genes, proteins, or RNAs in a rAAV packaging and/or producer cell line is maintained for at least one, at least two, at least three, at least four, at least five, at least ten, at least 20, at least 30, at least 40 or more passages of the rAAV packaging and/or producer cell line in culture.

Effect of Modulation on rAAV Production

Modulation of one or more genes and/or proteins in a rAAV packaging and/or producer cell line may result in an increase in the titer of rAAV. In some embodiments, modulation results in an increase in the titer of rAAV produced from the rAAV packaging and/or producer cell line is increased to about 1.5 to about 7-fold (e.g., about 1.5 to about 6.5, about 1.5 to about 6, about 1.5 to about 5.5, about 1.5 to about 5, about 1.5 to about 4.5, about 1.5 to about 4, about 1.5 to about 3.5, about 1.5 to about 3.0, about 1.5 to about 2.5, about 1.5 to about 2.0, about 2 to about 7, about 2.5 to about 7, about 3 to about 7, about 3.5 to about 7, about 4 to about 7, about 4.5 to about 7, about 5 to about 7, about 5.5 to about 7, about 6 to about 7, about 6.5 to about 7, or about 1.5, about 2.0, about 2.5, about 3.0, about 3.5, about 4.0, about 4.5, about 5.0, about 5.5, about 6.0, about 6.5, or about 7.0). In some embodiments, the titer of rAAV produced from the rAAV packaging and/or producer cell line is increased at least 2 fold, at least 3 fold, at least 4 fold, at least 5 fold, at least 6 fold, at least 7 fold, at least 8 fold, at least 9 fold, at least 10 fold, at least 15 fold, at least 20 fold or more. Any increase in the rAAV titer resulting from modulation of one or more genes and/or protein can be compared with the rAAV titer produced from a control parental cell line.

In some embodiments, modulation of one or more genes and/or proteins in a rAAV packaging and/or producer cell line may increase the rAAV titer production for at least 2 days, at least 5 days, at least 20 days, at least 30 days, at least 40 days, at least 50 days, at least 60 days, at least 70 days, at least 80 days, at least 90 days, at least 100 days or more.

Methods of Producing rAAV

In certain embodiments, the present disclosure describes a method of producing rAAV from rAAV packaging and/or producer cell lines that have been engineered to modulate the expression of one or more genes, proteins, or non-coding RNAs. In certain embodiments, rAAV is produced by infecting the cells of a rAAV producer cell line generated by delivering a rAAV vector to an engineered rAAV packaging cell line. In certain embodiments, rAAV is produced by infecting the cells of a rAAV producer cell line in which expression of one or more genes, proteins, or non-coding RNAs have been modulated. In certain embodiments, the production of rAAV from engineered rAAV packaging and/or producer cell line is enhanced as compared to a control parental cell line.

In certain embodiments, cells of the engineered packaging cell line are infected with a helper virus (e.g., adenovirus (AV) or herpes simplex virus), which allows the rAAV to replicate. In some embodiments, cells of the engineered producer cell line are infected with a helper virus (e.g., adenovirus (AV) or herpes simplex virus).

Methods of Harvesting rAAV rAAV particles may be obtained from engineered rAAV packaging and/or producer cells by lysing the cells. Lysis of engineered rAAV packaging and/or producer cells can be accomplished by methods that chemically or enzymatically treat the cells in order to release infectious viral particles. These methods include the use of nucleases such as benzonase or DNAse, proteases such as trypsin, or detergents or surfactants. Physical disruption, such as homogenization or grinding, or the application of pressure via a microfluidizer pressure cell, or freeze-thaw cycles may also be used. In certain embodiments, lysates from the engineered rAAV packaging and/or producer cells can be used to harvest rAAV particles.

In certain embodiments, cell culture supernatant may be collected from engineered rAAV packaging and/or producer cells without the need for cell lysis. In certain embodiments of the present disclosure, the engineered rAAV packaging and/or producer cells secrete rAAV particles that can be collected from the cell culture supernatant without the need for cell lysis. In certain embodiments, the engineered rAAV packaging and/or producer cell line has a higher rAAV titer than that of a control parental cell line such that more rAAV is harvested from the engineered rAAV packaging and/or producer cell line compared to the control parental cell line.

After harvesting rAAV particles, it may be necessary to purify the sample containing rAAV, to remove, for example, the cellular debris resulting from cell lysis. Methods of minimal purification of AAV particles are known in the art. Two exemplary purification methods are Cesium chloride (CsCl)- and iodixanol-based density gradient purification. Both methods are described in Strobel et al., Human Gene Therapy Methods., 26 (4): 147-157 (2015). Minimal purification can also be accomplished using affinity chromatography using, for example, AVB Sepharose affinity resin (GE Healthcare Bio-Sciences AB, Uppsala, Sweden). Methods of AAV purification using AVB Sepharose affinity resin are described in, for example, Wang et al., Mol Ther Methods Clin Dev., 2:15040 (2015). Following purification, rAAV particles may be filtered and stored at ≤−60° C.

In certain embodiments, the present disclosure provides a method of harvesting rAAV particles that are produced from an engineered rAAV packaging cell line after the cells have been co-infected with two different adenoviruses.

In certain embodiments, the present disclosure provides a method of harvesting rAAV particles that are produced after infection of a rAAV producer cell line generated from an engineered rAAV packaging cell line.

In certain embodiments, the present disclosure provides a method of harvesting rAAV particles that are produced after infection of an engineered rAAV producer cell line with a helper virus.

Quantification of rAAV Particles

Quantification of rAAV particles is complicated by the fact that AAV infection does not result in cytopathic effects in vitro, and therefore plaque assays cannot be used to determine infectious titers. rAAV particles can be quantified using a number of methods, however, including quantitative polymerase chain reaction (qPCR) (Clark et al., Hum. Gene Ther. 10, 1031-1039 (1999)), dot-blot hybridization (Samulski et al., J. Virol. 63, 3822-3828 (1989)), and by optical density of highly purified vector preparations (Sommer et al., Mol. Ther. 7, 122-128 (2003)). DNase-resistant particles (DRP) can be quantified by real-time quantitative gene expression reduced polymerase chain reaction (qPCR) (DRP-qPCR) in a thermocycler (for example, an iCycler iQ 96-well block format thermocycler (Bio-Rad, Hercules, CA)). Samples containing rAAV particles can be incubated in the presence of DNase I (100 U/ml; Promega, Madison, WI) at 37° C. for 60 min, followed by proteinase K (Invitrogen, Carlsbad, CA) digestion (10 U/ml) at 50° C. for 60 min, and then denatured at 95° C. for 30 min. The primer-probe set used should be specific to a non-native portion of the rAAV vector genome, for example, the poly(A) sequence of the protein of interest. The PCR product can be amplified using any appropriate set of cycling parameters, based on the length and composition of the primers, probe, and amplified sequence. Alternative protocols are disclosed in, for example, Lock et al., Human Gene Therapy Methods 25 (2): 115-125 (2014).

Viral genome amplification can also be measured using qPCR techniques similar to those described above. However, in order to quantify total genome amplification within producer cells, only intracellular samples are collected and the samples are not treated with DNase I in order to measure both packaged and unpackaged viral genomes. Viral genome amplification may be calculated on a per-host-cell basis by concomitantly measuring a host cell housekeeping gene, for example, RNase P.

The infectivity of rAAV particles can be determined using a TCID50 (tissue culture infectious dose at 50%) assay, as described for example in Zhen et al., Human Gene Therapy 15:709-715 (2004). In this assay, rAAV vector particles are serially diluted and used to co-infect a Rep/Cap-expressing cell line along with AV particles in 96-well plates. 48 hours post-infection, total cellular DNA from infected and control wells is extracted. rAAV vector replication is then measured using qPCR with transgene-specific probe and primers. TCID50 infectivity per milliliter (TCID50/ml) is calculated with the Karber equation, using the ratios of wells positive for AAV at 10-fold serial dilutions.

### Therapeutic Applications

The rAAV produced from the engineered rAAV packaging and/or producer cell lines described herein can be used, e.g., for gene therapy in mammals. The rAAV produced from the engineered cells described herein can be used for ex vivo and/or in vivo gene therapy applications. The rAAV produced from the engineered cells described herein can be used, e.g., to deliver small molecules (e.g., siRNAs or sgRNAs), peptides, and/or proteins.

In some embodiments, the rAAV generated from the engineered cell lines described herein can be used to treat a disease or a disorder in a human subject in need. In certain embodiments, the rAAV generated from the engineered cell lines described herein can be administered in conjunction with a pharmaceutically acceptable carrier.

Any suitable method or route can be used to administer a rAAV or a rAAV-containing composition produced from the engineered packaging and/or producer cell lines described herein. Routes of administration include, for example, systemic, oral, inhalation, intranasal, intratracheal, intraarterial, intraocular, intravenous, intramuscular, subcutaneous, intradermal, and other parenteral routes of administration. In some embodiments, the rAAV or a composition comprising a rAAV produced from the engineered packaging and/or producer cell line is administered intravenously.

Practice of the disclosure will be more fully understood from the foregoing examples, which are presented herein for illustrative purposes only, and should not be construed as limiting the disclosure in any way.

### Examples

### Example 1: Development of knockdown protocols

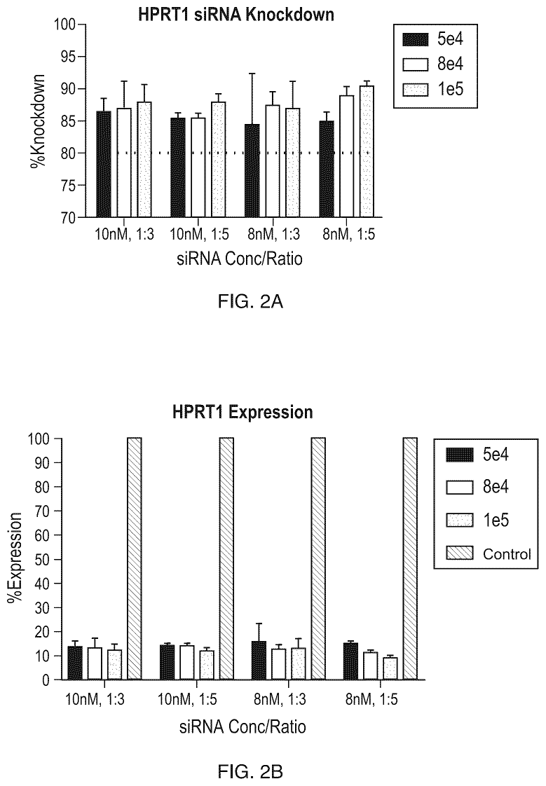

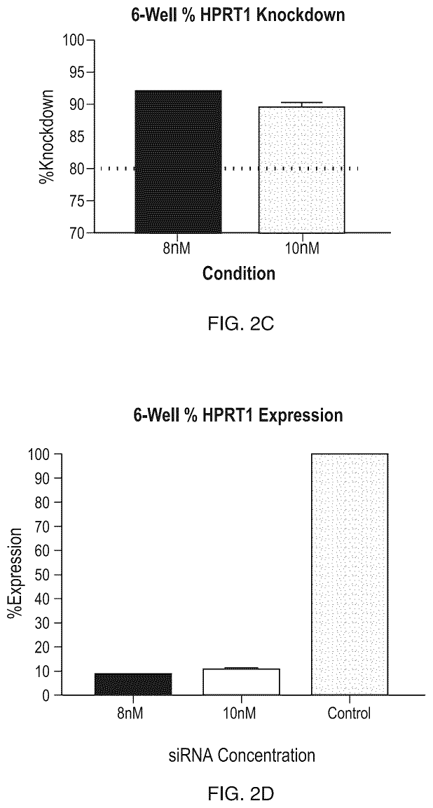

siRNA knockdown experiments were optimized and developed for 6-well and 24-well formats by knocking down the house keeping gene, HPRT1. The experiments performed in 24-wells were evaluated based on numerous factors such as seeding density, cell culture conditions (e.g., percent carbon dioxide (CO₂), percent of Fetal Bovine Serum (FBS)), ratio between transfection reagent (Lipofectamine® RNAiMax) and siRNA (“Ratio”), incubation time, and siRNA concentration. Commercially available siRNAs designed for HPRT1 gene knockdown were used to optimize the experimental conditions. HeLa producer cells were transfected with varying concentrations of siRNA using Lipofectamine® RNAiMax according to the manufacturer's instructions. Percent reduction of HPRT1 expression was determined by real time PCR. The optimized 24-well siRNA knockdown method was capable of knocking down the highly expressed gene, HPRT1, by more than 80% compared to baseline control. As shown in FIG. 2A-D, cells seeded at 1×10⁵ cells per well, 1:5 ratio between transfection reagent and siRNA, 8 nM of siRNA showed the highest knockdown efficiency. FIG. 2A shows the effect of varying siRNA concentrations/ratios used on the percent knockdown of HPRT1. FIG. 2B shows the effect of varying siRNA concentrations/ratios on the percent expression of HPRT1. For the 6-well protocol optimization, two different siRNA concentrations were tested. Seeding densities of 5×10⁴, 8×10⁴, and 1×10⁵ were tested for the data plotted in FIGS. 2A and 2B. FIG. 2C shows the effect of varying siRNA concentrations on the percent knockdown of HPRT1. FIG. 2D shows the effect of varying concentrations of siRNA on the percent expression of HPRT1. All experiments were performed in triplicate.

### Example 2: RNA Sequencing

Eight three-liter bioreactors were run at supplemented and non-supplemented production conditions across two different HeLaS3 producer cell lines. Two additional bioreactors were run without Adenovirus5 (Ad5) as uninfected controls. Table 3 lists details on bioreactor conditions and production levels.

TABLE 3
Bioreactor conditions and production levels.

| Condi- | Cell | Production | Seeding | Base | Supple- | |
|---|---|---|---|---|---|---|
| tion | Line | Level | Density | Media | ment(s) | Ad5 |
| 1 | 21C5 | No | 0.7 × 10⁶ | 90% DMEM/ | − | None |
| | | production | cells/mL | 10% Ex-Cell | | |
| | | control | | | | |
| 2 | 21C5 | Low | 0.7 × 10⁶ | 90% DMEM/ | − | 200 |
| | | | cells/mL | 10% Ex-Cell | | MOI |
| 3 | 21C5 | Low | 0.7 × 10⁶ | 90% DMEM/ | − | 200 |
| | | | cells/mL | 10% Ex-Cell | | MOI |
| 4 | 21C5 | Medium | 0.7 × 10⁶ | 90% DMEM/ | + | 200 |
| | | | cells/mL | 10% Ex-Cell | | MOI |
| 5 | 21C5 | Medium | 0.7 × 10⁶ | 90% DMEM/ | + | 200 |
| | | | cells/mL | 10% Ex-Cell | | MOI |
| 6 | 21C5 | High | 0.7 × 10⁶ | 90% DMEM/ | + | 200 |
| | | | cells/mL | 10% Ex-Cell | | MOI |
| 7 | 2B6 | No | 0.7 × 10⁶ | 90% DMEM/ | − | None |
| | | production | cells/mL | 10% Ex-Cell | | |
| | | control | | | | |
| 8 | 2B6 | Low | 0.7 × 10⁶ | 90% DMEM/ | − | 200 |
| | | | cells/mL | 10% Ex-Cell | | MOI |
| 9 | 2B6 | Medium | 0.7 × 10⁶ | 90% DMEM/ | + | 200 |
| | | | cells/mL | 10% Ex-Cell | | MOI |
| 10 | 2B6 | High | 0.7 × 10⁶ | 90% DMEM/ | + | 200 |
| | | | cells/mL | 10% Ex-Cell | | MOI |

Abbreviations used in Table 3: addition of one or more supplements is indicated by (+); absence of one or more supplements is indicated by (−); MOI-Multiplicity of infection.

Thirty hours post infection with Ad5, samples were pulled for RNA-Seq. Samples were washed once with PBS and cell pellets were stored at −80° C. until ready for shipment. RNA extraction and cDNA synthesis of extracted RNA were performed by methods well known in the art. Prior to sequencing, library preparation was done using commercially available RNA-Seq library preparation kits. RNA sequencing was done using commercially available Illumina sequencing platforms. Reads generated were mapped to human genome, Ad5 genome, and AAV2 genome using mapping methods well known in the art. Any reads mapped to Ad5 genome were discarded. Another round of sequencing was performed to enrich for reads mapped to the human genome. Differential analyses were performed using the data generated by RNA Sequencing (see Table 4).

TABLE 4
Differential analyses

| Differential | | |
|---|---|---|
| analysis # | Control Condition | Experimental Condition |
| 1 | PCL1; No Production | PCL1*; Production (N.S.) |
| 2 | PCL1; No Production | PCL2; No Production |
| 3 | PCL1; No Production | PCL1; Production (S) |
| 4 | PCL1; Production (N.S.) | PCL1; Production (S) |
| 5 | PCL1; Production (N.S.) | PCL1; Production (S) |
| 6 | PCL1; Production (N.S.) | PCL2; Production (N.S.) |
| 7 | PCL2; No Production | PCL2; Production (N.S.) |
| 8 | PCL2; No Production | PCL2; Production (S) |
| 9 | PCL2; No Production | PCL2; Production (S) |
| 10 | PCL1; Production (S) | PCL2; Production (S) |
| 11 | PCL2; Production (S) | PCL2; Production (S) |
| 12 | PCL1; Production (S) | PCL1; Production (S) |
| 13 | PCL1; Production (S) | PCL1; Production (S) |

*PCL1—producer cell line 1; PCL2—producer cell line 2; No Production—uninfected control cells; Production (N.S.)—Ad5 infected cells cultured under non-supplemented conditions; Production (S)—Ad5 infected cells cultured under supplemented conditions.

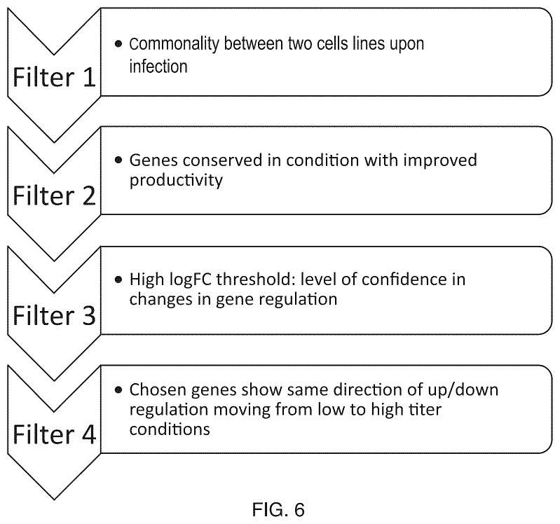

In this example, differential expression analysis was calculated as the log fold change (Log FC) in mRNA levels of the experimental condition compared to the control condition. Upregulated genes were expressed as a positive Log FC and downregulated genes were expressed as a negative Log FC. Differentially expressed genes having a p-value≤0.05 were considered statistically significant. Within each differential analysis hundreds to thousands of genes were significantly up or down regulated. A filtering criteria was established (see, e.g., FIG. 6) and applied to reduce the data set down to a manageable number of genes for evaluation. Gene sets were aligned and moved to the filter criteria as described in Example 6.

### Example 3: Validation of results obtained from RNA Sequencing by RT-qPCR

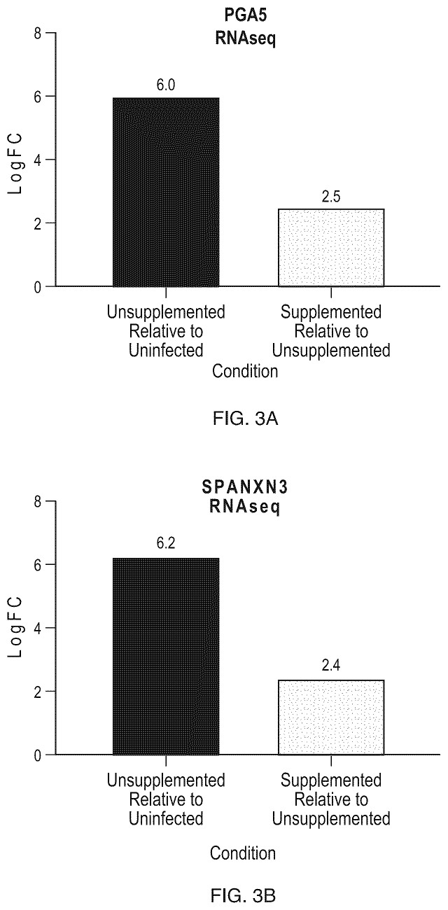

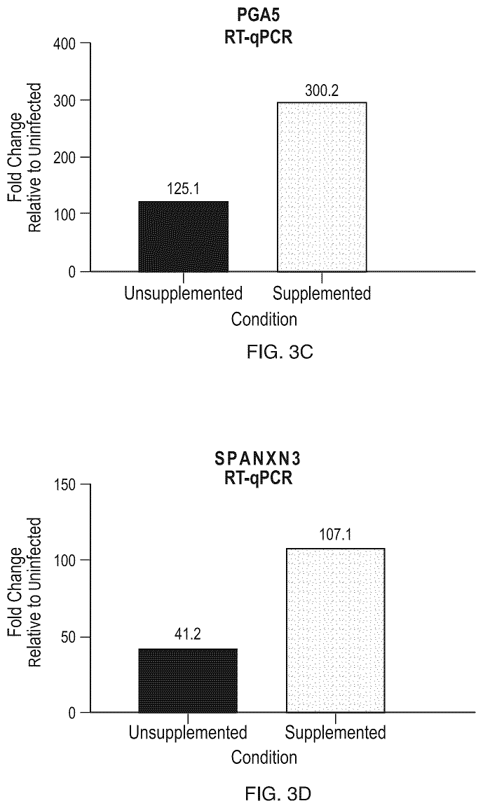

A small set of genes were selected for validation of RNA Sequencing data. RNA-Seq results were confirmed using an RT-qPCR assay following methods well-known in the art. The ΔΔCt method was used to analyze data. RT-qPCR independently confirmed the trends observed in the RNA-Seq data. FIG. 3A-B shows the log fold change values in gene expression obtained from bioinformatic analysis of RNA-Seq data for PGA5 (FIG. 3A) and SPANXN3 (FIG. 3B). X-axis shows the conditions (supplemented (differential analysis #5 as described in Table 4) versus non-supplemented (differential analysis #1 as described in Table 4)) in which the producer cell lines were grown and y-axis shows the log fold change (Log FC) in gene expression. Log fold change in PGA5 (FIG. 3A) and SPANXN3 (FIG. 3B) expression in cells cultured in unsupplemented cell culture medium is plotted relative to the corresponding gene expression in uninfected cells (cells not infected with a helper virus). Log fold change in PGA5 (FIG. 3A) and SPANXN3 (FIG. 3B) expression in cells cultured in supplemented cell culture medium is plotted relative to the corresponding gene expression in cells cultured in unsupplemented cell culture medium.

PGA5 and SPANXN3 gene expression in producer cell lines grown under supplemented and non-supplemented conditions was also evaluated by RT-qPCR by using methods well-known in the art. FIG. 3C-D show RT-qPCR fold change values in the expression of PGA5 (FIG. 3C) and SPANXN3 (FIG. 3D) in cells cultured in unsupplemented and supplemented cell culture medium, relative to uninfected cells (cells not infected with a helper virus). FIG. 3A-D show that data obtained from qPCR and RNA Sequencing follow the same trend.

### Example 4: Validation of Results Obtained from RNA Sequencing by RT-qPCR in Different Clones of a Producer Cell Line

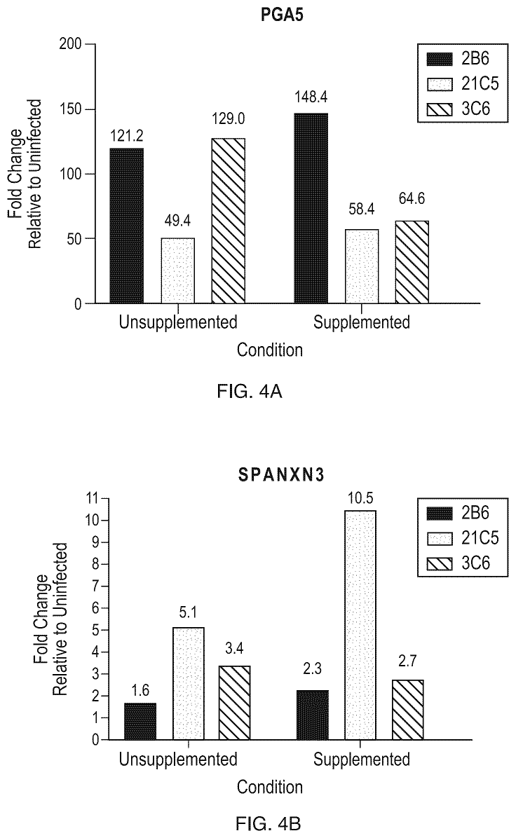

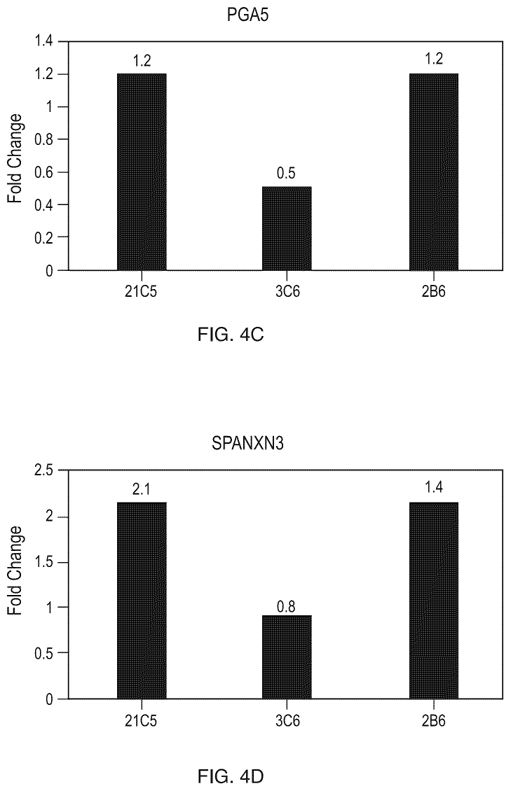

RNA Sequencing results were further validated by running RT-qPCR experiments on RNA extracted from different clones of a HeLa S3 producer cell line. FIG. 4A-B show the fold change values in PGA5 (FIG. 4A) and SPANXN3 (FIG. 4B) expression in producer cell line clones cultured in unsupplemented cell culture medium and supplemented cell culture medium relative to uninfected cells (cells not infected with a helper virus), as determined from RT-qPCR. 21C5, 3C6, and 2B6 represent different clones of the HeLa producer cell line. FIG. 4C-D show relative fold increase in PGA5 (FIG. 4C) and SPANXN3 (FIG. 4D) expression in producer cell line clones 21C5, 3C6, and 2B6 cultured in supplemented cell culture medium compared to the clones cultured in non-supplemented cell culture medium. These results further validate bioinformatic RNA Sequencing and RT-qPCR data described in Example 3.

### Example 5: Effect of gene knockdown on rAAV titer

Knockdown experiments were performed by individually knocking down genes in HeLa producer cell lines based on the optimized protocol discussed in Example 1. siRNA nucleotide sequences were designed for each gene (see Table 1).

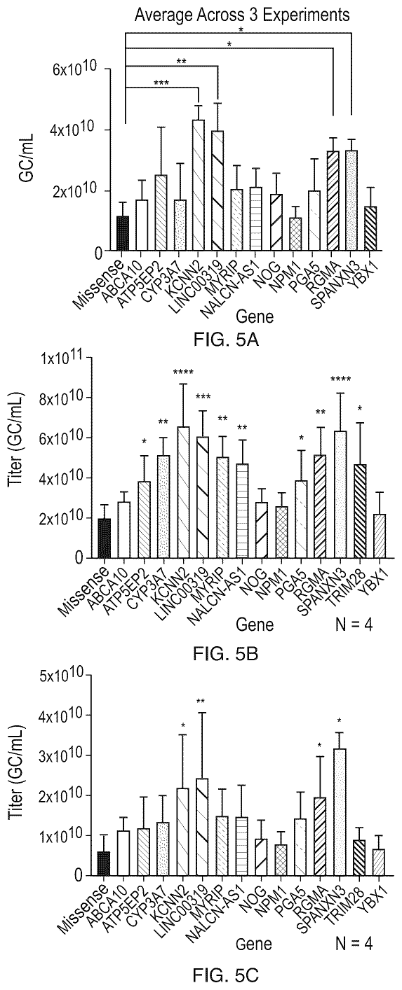

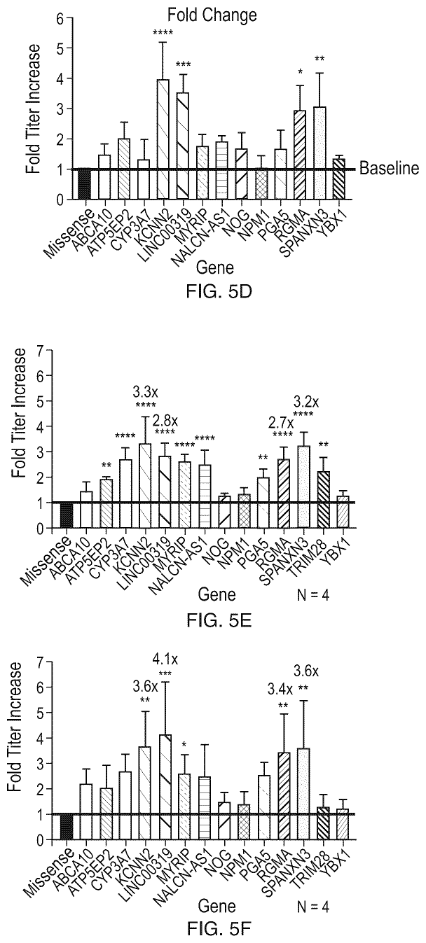

The condition settled upon as a 1×10⁵ seeding density with 8 nM siRNA and a siRNA:RNAiMAX ratio of 1:5. AAV production was induced 24 hours post reduction of expression of genes, and rAAV was harvested 72 hours post infection. Titer was determined for each sample and compared to a non-targeting missense siRNA control. This experiment was performed independently three times, results were averaged, and statistical analysis was performed. FIGS. 5A-5C show the result of siRNA of individual genes in producer cell lines 1-3, respectively, on absolute rAAV titer (GC/mL; GC=genome copies). FIGS. 5D-5F show the fold increase on rAAV titer by siRNA of individual genes in different producer cell lines 1-3, respectively.

As shown in FIGS. 5A-5F, reduction of expression of KCNN2, LINC00319, RGMA, or SPANXN3 in producer cell lines resulted in statistically significant 2-4-fold higher rAAV titers over missense control. Across three producer cell lines, these four genes show a statistically relevant positive impact on titer when knocked down. These results indicate that these genes are excellent targets for more permanent modifications, such as CRISPR/Cas9 knockouts.

### Example 6: Gene Filtering Methodology

For filter 1, the genes from differential analysis 1 and 7 (as described in Table 3) were aligned. The differential analysis for 1 and 7 defined genes that are up or down-regulated upon the addition of adenovirus 5 in non-supplemented conditions. Analysis 1 looked at the cells from 21C5 producer cell line (producer cell line 1, PCL1). Analysis 7 looked at the cells from 2B6 (producer cell line 2, PCL2). List of genes after this filter 1 identified genes that were not cell line specific, and this alignment provided a total of 9149 genes that were in common between the two producer cell lines.

For filter 2, the genes from filter 1 were the aligned with genes present in differential analysis 5. Analysis 5 looked at genes that were up and down regulated in cells from 21C5 producer cell line (PCL1) in supplemented conditions compared to non-supplemented conditions. The purpose of this differential analysis was to define the effects of production under supplemented conditions in regards to production under non-supplemented conditions. The purpose of aligning the gene set from filter 1 with differential analysis 5 was to identify genes in the improved productivity conditions that are 1) not a byproduct of the improved production conditions 2) potentially relevant for two different cell lines. After alignment, 374 genes were moved forward.

For filter 3, only genes that had a large LogFc threshold of >2 Log FC± were moved forward. This was done to ensure a high level of up/down regulation in the genes being moved forward, and to give a degree of confidence that the genes selected were not artifacts of the RNA-Seq. After the filter, 77 genes were moved forward.

For filter 4, only genes that showed both up-regulation in differential analysis 1 and differential analysis 5 or down-regulation in differential analysis 1 and differential analysis 5 were kept. For example, one of the 77 genes must show up-regulation from differential analysis 1 and further up-regulation in differential analysis 5 or down-regulation in differential analysis 1 and further down-regulation in differential analysis 5. The purpose of this filter was to ensure that, for the genes being evaluated, high titer conditions were not having an antagonistic effect on that particular gene's regulation compared to low titer conditions. After the filter eleven genes were left to be evaluated. An illustrative flow-chart showing an exemplary gene filtering methodology is shown in FIG. 6 (abbreviation used: Log FC=Log fold change).

Table 5 provides Log2FC data from each comparison during the process of filtering down important genes for productivity.

TABLE 5
Log2FC data

| | Differential | Differential | Differential |
|---|---|---|---|
| Gene | Analysis 7 | Analysis 1 | Analysis 5 |
| ATP5EP2 | 2.409 | −1.1 | −7.511 |
| LINC00319 | −4.382 | −1.432 | −6.58 |
| CYP3A7 | −8.018 | −3.149 | −2.814 |
| ABCA10 | −4.257 | −2.025 | −3.131 |
| NOG | −5.585 | −1.468 | −2.814 |
| SPANXN3 | 4.99 | 6.238 | 2.423 |
| PGA5 | 8.153 | 6.019 | 2.519 |
| MYRIP | 2.045 | 2.771 | 2.175 |
| KCNN2 | 3.656 | 2.807 | 2.066 |
| NALCN- | 4.558 | 2.639 | 2.024 |
| AS1 | | | |
| RGMA | 2.764 | 2.303 | 2.03 |

### Example 7: Gene Knockout of KCNN2

In this example, two existing, highly optimized monoclonal HeLa producer cell lines (PCLs)-2H5 and 7B12-were genetically modified to knockout the KCNN2 gene (previously identified in the RNA-seq screen described herein), which encodes a calcium-activated potassium channel protein, SK2.

KCNN2 was knocked out in 2H5 or 7B12 HeLa cells using an eGFP selectable marker. Suspected KCNN2 knockouts were enriched for eGFP expression and seeded in 96-well plates. Cell colonies were allowed to form, genomic DNA was harvested, and PCR was performed to amplify the region containing the knockout. The PCR product was Sanger sequenced and the sequencing files were analyzed for the presence of insertion/deletions. 2H5 and 7B12 clones with a high likelihood of knockout were scaled-up for further testing.

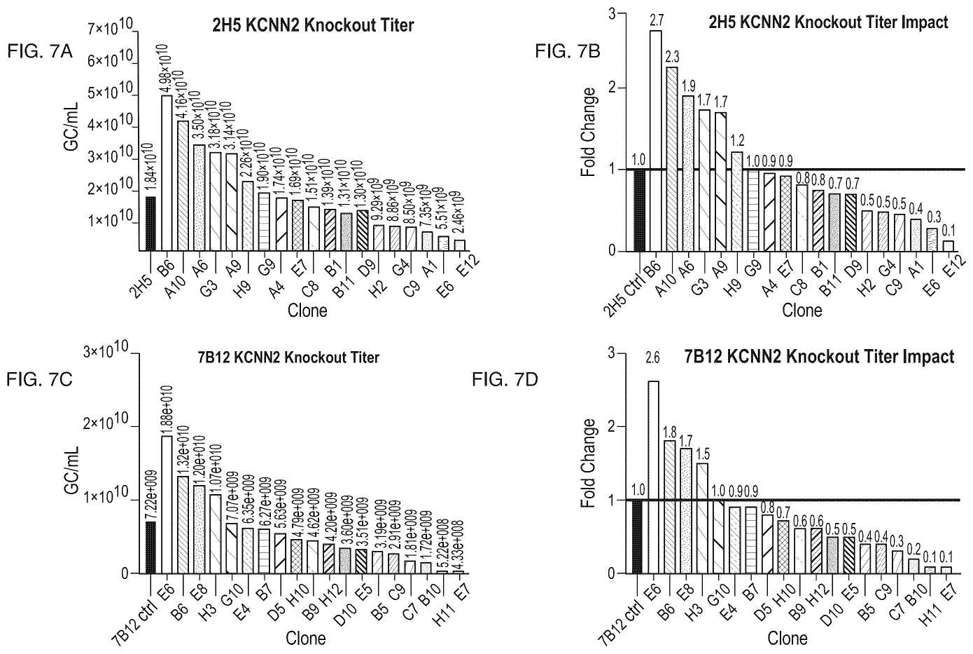

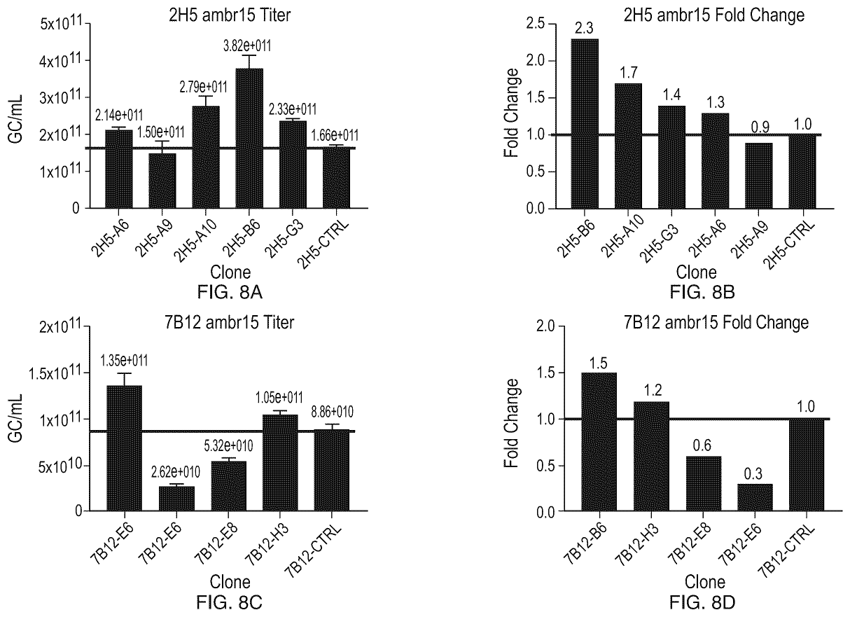

Top clones were transferred into serum free, suspension culture. Clone productivity compared to the parental line was assessed through a 24 deep well rAAV production. Clones were seeded at 2×10⁵ cells/mL in 3 mL of culture and infected with Ad5 at a multiplicity of infection (MOI) of 50. Four days post infection, rAAV was harvested and assessed for titer. Fold increase in titer was normalized to the parental control. The best clones displayed 1.5-2.7 fold increases in titer compared to the control samples. 2H5 titers ranged from 2.46×10⁹-4.98×10¹⁰ GC/mL (FIG. 7A). When titers were normalized to the parental control, fold increases were seen ranging from 1.2-2.7 fold (FIG. 7B). 7B12 titers ranged from 4.33×10⁸-1.88×10¹⁰ GC/mL (FIG. 7C). When titers were normalized to the parental control, fold increases were seen ranging from 1.5-2.6 fold (FIG. 7D). Clones with a minimum of 1.5 fold increase were then scaled into shake flask culture and inoculated into the ambr® 15 for high seeding density, supplemented rAAV production. Cells were seeded at 1.5×10⁶ cells/mL and infected with Ad5 at an MOI of 50. rAAV was harvested four days post infection and assessed for titer. Fold increase in titer was normalized to the parental control. The best clones displayed 1.5-2.3 fold increases in titer compared to the control samples. 2H5 titers ranged from 1.5×10¹¹-3.82×10¹¹ GC/mL (FIG. 8A). When titers were normalized to the parental control, fold increases were seen ranging from 1.3-2.3 fold (FIG. 8B). 7B12 titers ranged from 2.62×10¹⁰-1.35×10¹¹ GC/mL (FIG. 8C). When titers were normalized to the parental control, fold increases were seen ranging from 1.2-1.5 fold (FIG. 8D).

These data establish that reducing or eliminating the expression of one or more genes described herein (e.g., via gene knockout) in AAV-producing cells can be employed to increase the production of rAAV from engineered cells.

### Example 8: Multi-Combinatorial siRNA Knockdowns

In this example, multi-combinational knockdown of genes previously identified in the RNA-seq screen described herein using siRNA was performed to determine if targeting multiple genes simultaneously would produce an additive effect on titer.

Multi-combinational knockdowns were performed using a modification of the methods described in Example 5. Briefly, cells were transfected using 8 nM of each siRNA and maintaining a ratio of siRNA:RNAiMAX of 1:5. AAV production was induced 24 hours post reduction of expression of genes, and rAAV was harvested 72 hours post infection. Titer was determined for each sample and compared to a non-targeting missense siRNA control.

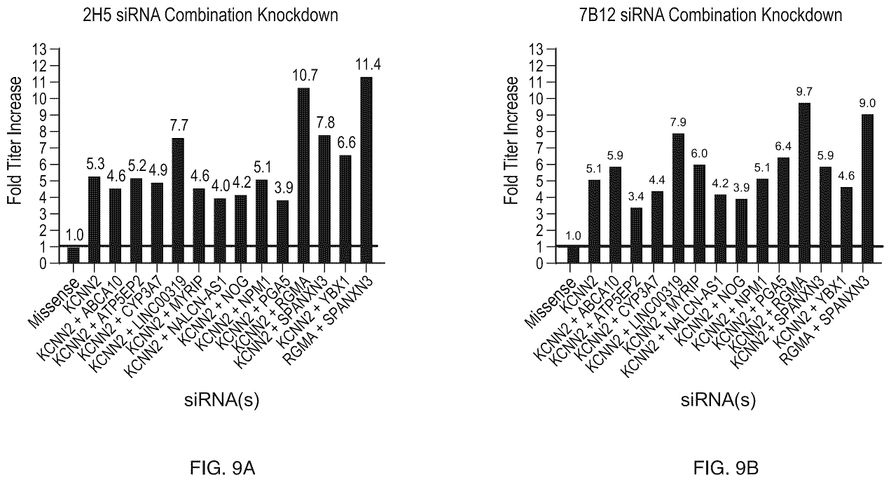

In this example, KCNN2 was knocked down in combination with the panel of other siRNAs previously described. Additionally, RGMA and SPANXN3 were knocked down in combination with each other. In 2H5, combination knockdowns displayed a range of titer increases from 4.6-11.4 fold compared to a missense control (FIG. 9A). In 7B12, combination knockdowns displayed a range of titer increases 3.4-9.7 fold compared to the missense control (FIG. 9B). Every combination displayed an increase in titer; however, not all combinations were an improvement over knocking down KCNN2 alone. KCNN2 knockdown led to a 5.3 fold increase in 2H5 (FIG. 9A) and a 5.1 fold increase in 7B12 (FIG. 9B).

These data establish that additional increases in the production of rAAV can be gained through direct targeting of multiple genomic regions in established high rAAV titer producing monoclonal PCLs.

### NUMBERED EMBODIMENTS

1. A recombinant adeno-associated virus (rAAV) packaging and/or producer cell line comprising cells in which the expression of ATP5EP2, LINC00319, CYP3A7, ABCA10, NOG, RGMA, SPANXN3, PGA5, MYRIP, KCNN2, and/or NALCN-AS1 is reduced compared to control parental cells.

2. The packaging and/or producer cell line according to embodiment 1, comprising cells in which expression of KCNN2, LINC00319, RGMA, and SPANXN3 is reduced compared to control parental cells.

3. The packaging and/or producer cell line according to embodiment 1 or 2, wherein the expression is reduced using a nuclease, a double stranded RNA (dsRNA), a small interfering RNA (siRNA), a small hairpin RNA (shRNA), a microRNA (miRNA), or an antisense RNA oligonucleotide (ASO).

4. The packaging and/or producer cell line according to any one of embodiment 1-3, wherein the expression is reduced with an siRNA comprising a nucleotide sequence selected from any one of SEQ ID NOs: 1-11.

5. The packaging and/or producer cell line according to embodiment 4, wherein expression of ATP5EP2 is reduced, and the siRNA comprises the nucleotide sequence of SEQ ID NO: 1 in the sense strand and the nucleotide sequence of SEQ ID NO: 32 in the anti-sense strand.

6. The packaging and/or producer cell line according to embodiment 4, wherein expression of LINC00319 is reduced, and the siRNA comprises the nucleotide sequence of SEQ ID NO: 2 in the sense strand and the nucleotide sequence of SEQ ID NO: 33 in the anti-sense strand.

7. The packaging and/or producer cell line according to embodiment 4, wherein expression of CYP3A7 is reduced, and the siRNA comprises the nucleotide sequence of SEQ ID NO: 3 in the sense strand and the nucleotide sequence of SEQ ID NO: 34 in the anti-sense strand.

8. The packaging and/or producer cell line according to embodiment 4, wherein expression of NOG is reduced, and the siRNA comprises the nucleotide sequence of SEQ ID NO: 4 in the sense strand and the nucleotide sequence of SEQ ID NO: 35 in the anti-sense strand.

9. The packaging and/or producer cell line according to embodiment 4, wherein expression of SPANXN3 is reduced, and the siRNA comprises the nucleotide sequence of SEQ ID NO: 5 in the sense strand and the nucleotide sequence of SEQ ID NO: 36 in the anti-sense strand.

10. The packaging and/or producer cell line according to embodiment 4, wherein expression of MYRIP is reduced, and the siRNA comprises the nucleotide sequence of SEQ ID NO: 6 in the sense strand and the nucleotide sequence of SEQ ID NO: 37 in the anti-sense strand.

11. The packaging and/or producer cell line according to embodiment 4, wherein expression of KCNN2 is reduced, and the siRNA comprises the nucleotide sequence of SEQ ID NO: 7 in the sense strand and the nucleotide sequence of SEQ ID NO: 38 in the anti-sense strand.

12. The packaging and/or producer cell line according to embodiment 4, wherein expression of NALCN-AS1 is reduced, and the siRNA comprises the nucleotide sequence of SEQ ID NO: 8 in the sense strand and the nucleotide sequence of SEQ ID NO: 39 in the anti-sense strand.

13. The packaging and/or producer cell line according to embodiment 4, wherein expression of RGMA is reduced, and the siRNA comprises the nucleotide sequence of SEQ ID NO: 9 in the sense strand and the nucleotide sequence of SEQ ID NO: 40 in the anti-sense strand.

14. The packaging and/or producer cell line according to embodiment 4, wherein expression of PGA5 is reduced, and the siRNA comprises the sequence of SEQ ID NO: 10 in the sense strand and the sequence of SEQ ID NO: 41 in the anti-sense strand.

15. The packaging and/or producer cell line according to embodiment 4, wherein expression of ABCA10 is reduced, and the siRNA comprises the sequence of SEQ ID NO: 11 in the sense strand and the sequence of SEQ ID NO: 42 in the anti-sense strand.

16. The packaging and/or producer cell line according to embodiment 3, wherein the nuclease is selected from the group consisting of a Zinc Finger nuclease (ZFN), a meganuclease, a transcription activator-like effector nuclease (TALEN), or a clustered regularly interspaced short palindromic repeats (CRISPR) associated protein.

17. The packaging and/or producer cell line according to one of embodiments 1-16, wherein the expression is reduced using CRISPR genome editing.

18. The packaging and/or producer cell line according to embodiment 17, wherein the expression is reduced using a guide RNA pair, wherein each guide RNA:
(a) comprises a sequence selected from the nucleotide sequences of SEQ ID NOs: 12-15, and/or
(b) targets a target DNA sequence selected from any one of the nucleotide sequences of SEQ ID NO: 16-31.

19. The packaging and/or producer cell line according to embodiment 18, wherein the gRNA pair is used to target KCNN2 and comprises a first gRNA molecule comprising the sequence of SEQ ID NO: 12 and a second gRNA molecule comprising the sequence of SEQ ID NO: 13.

20. The packaging and/or producer cell line according to embodiment 18, wherein the gRNA pair is used to target KCNN2 and comprises a first gRNA molecule comprising the sequence of SEQ ID NO: 14 and a second gRNA molecule comprising the sequence of SEQ ID NO: 15.

21. The packaging and/or producer cell line of embodiment 19 or 20, wherein each gRNA molecule is a 2′ O-methyl analog comprising 3′ phosphorothioate internucleotide linkages in the terminal three nucleotides on either or both its 5′ and 3′ ends.

22. The packaging and/or producer cell line according to any one of embodiments 1-21, wherein the gene expression is eliminated compared to control parental cells.

23. The packaging and/or producer cell line according to any one of embodiments 1-22, wherein the cell line is a human cell line.

24. The packaging and/or producer cell line according to embodiment 23, wherein the human cell line is a HeLa cell line or a human embryonic kidney (HEK) 293 cell line.

25. The cell line according to any one of embodiments 1-24, wherein the cell line is a rAAV packaging cell line.

26. The cell line according to any one of embodiments 1-24, wherein the cell line is a rAAV producer cell line.

27. The cell line according to embodiment 26, wherein the titer of rAAV is increased about 1.5 to about 7 fold compared to the titer of rAAV produced from a cell line comprising the control parental cells.

28. A lysate of the cell line according to any one of embodiments 1-27.

29. A cell culture supernatant from a cell line according to any one of embodiments 1-27.

30. A method of generating a producer cell line, the method comprising delivering a recombinant adeno-associated virus (rAAV) vector to cells of a packaging cell line according to embodiment 25.

31. A method of producing rAAV, the method comprising infecting the cells of a producer cell line generated by the method of embodiment 30 with a helper virus.

32. A method of producing rAAV, the method comprising infecting the cells of a producer cell line according to embodiment 26 with a helper virus.

33. A method of embodiment 31 or 32, wherein the rAAV is harvested from the producer cell line.

34. A method of any one of embodiments 31 to 33, wherein the production of rAAV is enhanced as compared to a control parental cell line.

35. A method of identifying one or more genes relevant to the production of rAAV, the method comprising:
adding one or more supplements that increase the rAAV titer in a cell line;
measuring the global gene expression across the transcriptome in supplemented and non-supplemented cell lines;
obtaining a list of genes that are differentially expressed between supplemented and non-supplemented cell lines; and
identifying one or more genes that are relevant to the production of rAAV.

36. The method of embodiment 35, wherein the one or more supplements added to the cell line comprise dexamethasone, hydrocortisone, prednisolone, methylprednisolone, betamethasone, cortisone, prednisone, budesonide, or triamcinolone.

37. A method of producing a rAAV packaging and/or producer cell line to promote increased production of rAAV, the method comprising modulating the expression of one or more genes identified using the method of embodiment 35.

38. The method of any one of embodiments 35-37, wherein the cell line is a rAAV packaging cell line.

39. The method of any one of embodiments 35-37, wherein the cell line is a rAAV producer cell line.

40. The method of embodiment 39, wherein the rAAV producer cell line increases rAAV titer at least 1.5 fold greater than the rAAV titer produced by a rAAV producer cell line without the modulation of expression of the corresponding one or more genes.

41. The method of any one of embodiments 37-40, wherein modulating the expression comprises reduction of expression of one or more genes.

42. The method of any one of embodiments 37-40, wherein modulating the expression comprises elimination of expression of one or more genes.

43. The method of any one of embodiments 30-42, wherein the cell line is a human cell line.

44. The method of embodiment 43, wherein the human cell line is a HeLa cell line or a human embryonic kidney (HEK) 293 cell line.

45. A recombinant adeno-associated virus (rAAV) packaging and/or producer cell line comprising cells which have been engineered to reduce the expression and/or activity of a gene product expressed from ATP5EP2, LINC00319, CYP3A7, ABCA10, NOG, RGMA, SPANXN3, PGA5, MYRIP, KCNN2, and/or NALCN-AS1 as compared to corresponding unmodified parental cells.

46. The rAAV packaging and/or producer cell line of embodiment 45, wherein the expression and/or activity of a gene product expressed from ATP5EP2, LINC00319, CYP3A7, ABCA10, NOG, RGMA, SPANXN3, PGA5, MYRIP, KCNN2, and/or NALCN-AS1 is reduced indefinitely or permanently.

47. The rAAV packaging and/or producer cell line of embodiment 46, wherein the cell line has been engineered to comprise a gene disruption or a partial or complete gene deletion in at least one of ATP5EP2, LINC00319, CYP3A7, ABCA10, NOG, RGMA, SPANXN3, PGA5, MYRIP, KCNN2, and/or NALCN-AS1.

48. The rAAV packaging and/or producer cell line of embodiment 47, wherein the cell line has been engineered to comprise a gene disruption in at least one of ATP5EP2, LINC00319, CYP3A7, ABCA10, NOG, RGMA, SPANXN3, PGA5, MYRIP, KCNN2, and/or NALCN-AS1.

49. The rAAV packaging and/or producer cell line of embodiment 47, wherein the cell line has been engineered to comprise a gene disruption in at least two genes selected from ATP5EP2, LINC00319, CYP3A7, ABCA10, NOG, RGMA, SPANXN3, PGA5, MYRIP, KCNN2, and NALCN-AS1.

50. The rAAV packaging and/or producer cell line of embodiment 47, wherein the cell line has been engineered to comprise a partial or complete gene deletion in at least one of ATP5EP2, LINC00319, CYP3A7, ABCA10, NOG, RGMA, SPANXN3, PGA5, MYRIP, KCNN2, and/or NALCN-AS1.

51. The rAAV packaging and/or producer cell line of embodiment 47, wherein the cell line has been engineered to comprise a partial or complete gene deletion in at least two genes selected from ATP5EP2, LINC00319, CYP3A7, ABCA10, NOG, RGMA, SPANXN3, PGA5, MYRIP, KCNN2, and NALCN-AS1.

52. A recombinant adeno-associated virus (rAAV) packaging and/or producer cell line, wherein said cell line exhibits reduced expression and/or activity of a polypeptide or polyribonucleotide expressed from at least one of ATP5EP2, LINC00319, CYP3A7, ABCA10, NOG, RGMA, SPANXN3, PGA5, MYRIP, KCNN2, and NALCN-AS1 as compared to a corresponding parental cell line.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A recombinant adeno-associated virus (rAAV) packaging and/or producer cell line comprising a plurality of engineered cells which have reduced expression of a gene product expressed from Repulsive Guidance Molecule BMP Co-Receptor A (RGMA) as compared to corresponding unmodified parental cells.

2. The rAAV packaging and/or producer cell line according to claim 1, wherein the expression of RGMA in the plurality of engineered cells has been reduced using
a) a nuclease selected from the group consisting of a Zinc Finger nuclease (ZFN), a meganuclease, a transcription activator-like effector nuclease (TALEN), and a clustered regularly interspaced short palindromic repeats (CRISPR) associated protein, or
b) CRISPR genome editing.

3. The rAAV packaging and/or producer cell line according to claim 1, wherein the expression of RGMA in the plurality of engineered cells has been reduced using a guide RNA (gRNA) pair, wherein each gRNA targets a target DNA sequence selected from any one of the nucleotide sequences of SEQ ID NOs: 20-23.

4. The rAAV packaging and/or producer cell line of claim 3, wherein each gRNA molecule is a 2′ O-methyl analog comprising 3′ phosphorothioate internucleotide linkages in the terminal three nucleotides on either or both its 5′ and 3′ ends.

5. The rAAV packaging and/or producer cell line according to claim 1, wherein the gene expression of RGMA in the plurality of engineered cells is eliminated compared to corresponding unmodified parental cells.

6. The rAAV packaging and/or producer cell line according to claim 1, wherein the plurality of engineered cells further have reduced expression of a gene product expressed from Potassium Calcium Activated Channel Subfamily N Member 2 (KCNN2), ATP Synthase F1 Subunit Epsilon Pseudogene 2 (ATP5EP2), Long Intergenic Non-Protein Coding RNA 319 (LINC00319), Cytochrome P450 Family 3 Subfamily A Member 7 (CYP3A7), ATP Binding Cassette Subfamily A Member 10 (ABCA10), Noggin (NOG), SPANX Family Member N3 (SPANXN3), Pepsinogen A5 (PGA5), Myosin VIIA And Rab Interacting Protein (MYRIP), and/or NALCN Antisense RNA 1 (NALCN-AS1) compared to corresponding unmodified parental cells.

7. The rAAV packaging and/or producer cell line of claim 6, wherein the plurality of engineered cells comprise a gene disruption in at least one of KCNN2, ATP5EP2, LINC00319, CYP3A7, ABCA10, NOG, SPANXN3, PGA5, MYRIP, and NALCN-AS1.

8. The rAAV packaging and/or producer cell line of claim 6, wherein the plurality of engineered cells comprise a partial gene deletion in at least one of KCNN2, ATP5EP2, LINC00319, CYP3A7, ABCA10, NOG, SPANXN3, PGA5, MYRIP, and NALCN-AS1.

9. The rAAV packaging and/or producer cell line of claim 6, wherein the plurality of engineered cells comprise a complete gene deletion in at least one of KCNN2, ATP5EP2, LINC00319, CYP3A7, ABCA10, NOG, SPANXN3, PGA5, MYRIP, and NALCN-AS1.

10. The rAAV packaging and/or producer cell line of claim 1, wherein the cell line is a HeLa cell line or a human embryonic kidney (HEK) 293 cell line.

11. The rAAV packaging and/or producer cell line according to claim 1, wherein the cell line is a rAAV packaging cell line.

12. The rAAV packaging and/or producer cell line according to claim 1, wherein the cell line is a rAAV producer cell line.

13. A lysate of the rAAV producer cell line or a cell culture supernatant from a rAAV producer cell line according to claim 12.

14. A method of generating a rAAV producer cell line, the method comprising delivering a rAAV vector to the plurality of cells of the rAAV packaging cell line according to claim 11.

15. A method of producing rAAV, the method comprising infecting the cells of a rAAV producer cell line generated by the method of claim 14 with a helper virus.

16. The method of claim 15, wherein the production of rAAV is enhanced as compared to corresponding unmodified parental cells.

17. The method of claim 15, wherein the rAAV producer cell line is a HeLa cell line or a human embryonic kidney (HEK) 293 cell line.
